SEQUENCE LISTING (7 sequences)

SEQ ID NO: 1 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTSPRSHRSDGEFSPHSHYSDSDEAS

SEQ ID NO: 2 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTAPRSHRSDGEFSPHSHYSDSDEAS

SEQ ID NO: 3 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTSPRAHRSDGEFSPHSHYSDSDEAS

SEQ ID NO: 4 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTAPRSHRSDGEFSPHSHYSDSDEAS

SEQ ID NO: 5 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTSPRAHRSDGEFSPHSHYSDSDEAS

SEQ ID NO: 6 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENSKTAPRAHRSDGEFSPHSHYSDSDEAS

SEQ ID NO: 7 - protein, 354 aa, Artificial Sequence
  Synthetic Construct
MSRLLHAEEWAEVKELGDHHRQPQPHHLPQPPPPPQPPATLQAREHPVYPPELSLLDSTDPRAWLAPTLQGICTARAAQYLLHSPELGASEAAAPRDEVDGRGELVRRSSGGASSSKSPGPVKVREQLCKLKGGVVVDELGCSRQRAPSSKQVNGVQKQRRLAANARERRRMHGLNHAFDQLRNVIPSFNNDKKLSKYETLQMAQIYINALSELLQTPSGGEQPPPPPASCKSDHHHLRTAASYEGGAGNATAAGAQQASGGSQRPTPPGSCRTRFSAPASAGGYSVQLDALHFSTFEDSALTAMMAQKNLSPSLPGSILQPVQEENAKTAPRAHRSDGEFSPHSHYSDSDEAS

(19) United States
(12) Patent Application Publication
(10) Pub. No.: US 2024/0131190 A1
(43) Pub. Date: Apr. 25, 2024

(54) METHODS AND COMPOSITIONS FOR REDUCING NUCLEIC ACID VECTOR-INDUCED TOXICITY IN THE INNER EAR

(71) Applicant: Decibel Therapeutics, Inc., Boston, MA (US)

(72) Inventors: Adam PALERMO, Somerville, MA (US); Ning PAN, Newton, MA (US); Gabriela PREGERNIG, Cambridge, MA (US); Jonathon WHITTON, Cambridge, MA (US); Xichun ZHANG, Belmont, MA (US)

(21) Appl. No.: 18/278,053

(22) PCT Filed: Feb. 22, 2022
(86) PCT No.: PCT/US2022/017310

§ 371 Date: Aug. 20, 2023

Related U.S. Application Data
(60) Provisional application No. 63/152,302, filed on Feb. 22, 2021.

(51) Int. Cl. A61K 48/00; A61K 9/00; C12N 9/22; C12N 15/113

(52) U.S. Cl. CPC A61K 48/0058; A61K 9/0046; C12N 9/22; C12N 15/1138; C12N 2310/14; C12N 2310/20

(57) ABSTRACT

The disclosure features compositions and methods for the treatment of inner ear dysfunction, such as hearing loss or vestibular dysfunction, that reduce inflammatory or cell-mediated immune toxicity in the inner ear, thereby improving transduction and therapeutic efficacy. The disclosure provides a variety of compositions that include a nucleic acid vector that contains a polynucleotide encoding a therapeutic agent operably linked to a ubiquitous promoter and inhibitor of inflammatory or immune cell signaling. The disclosed compositions and methods can be used to increase expression of the therapeutic agent in a subject, such as a human subject suffering from an inner ear dysfunction, while minimizing undesirable immune activation resulting from off-target expression of the target protein in immune cells of the inner ear.

### SEQUENCE LISTING

The instant application contains a Sequence Listing which has been submitted electronically in ASCII format and is hereby incorporated by reference in its entirety. The ASCII copy, created on Feb. 22, 2022, is named 51124-092WO2_Sequence_Listing_2_22_22_ST25 and is 22,211 bytes in size.

### BACKGROUND

Gene therapy has recently emerged as a promising approach for treating disorders of the inner ear, such as hearing loss and vestibular dysfunction, as it can be used to treat the genetic causes of these disorders, induce the expression of genes that encode therapeutic proteins, and may lead to the preservation or restoration of hearing with more natural sound perception than a cochlear implant. However, mutations that cause hearing loss and/or vestibular dysfunction have been identified in a variety of different cell types in the inner ear, thus, gene therapy approaches may require the use of nucleic acid vectors having tropism for multiple cell types of the inner ear. Such pantropism may be achieved using nucleic acid vectors containing ubiquitous promoter sequences capable of driving transgene expression in multiple cell types. Ubiquitous promoters can also be used to induce high levels of transgene expression. However, the use of AAV vectors containing ubiquitous promoters such as CMV and CAG has been found to cause severe toxicity in other sensory systems, such as the retina. Therefore, there is a need to develop approaches for gene therapy using nucleic acid vectors that contain ubiquitous promoters that do not cause undesirable toxicity.

### SUMMARY OF THE INVENTION

The present disclosure is based on the inventors' discovery that adeno-associated viral (AAV) vectors encoding a transgene under the control of a ubiquitous promoter induced deleterious immune toxicity associated with increased expression of apoptosis genes, off-target expression of the transgene in immune cells of the inner ear, activation of antigen-directed immunity mediated by major histocompatibility complex (MHC) genes, T cell genes, and macrophage genes, increases in the number of immune cells (e.g., monocytes and dendritic cells) in the inner ear, and elevated ligand-receptor interactions involving apoptosis pathways, such as, e.g., tumor necrosis family receptor super family (TNFRSF)-mediated interactions involving neutrophils and granulocytes. Accordingly, the present disclosure provides compositions and methods for transducing cell types of the inner ear using a nucleic acid vector (e.g., an AAV vector) containing ubiquitous promoter (e.g., operably linked to a polynucleotide encoding a therapeutic agent, such as a polynucleotide encoding a protein, an inhibitory RNA, or a nuclease) in combination with an inhibitor of inflammatory or immune signaling. The compositions disclosed herein can be administered to a subject, such as a human subject, to promote the expression of a polynucleotide operably linked to the ubiquitous promoter, such as a polynucleotide corresponding to a gene that promotes or improves inner ear cell function, regeneration, maintenance, development, proliferation, or survival, or a polynucleotide that corresponds to a wild-type version of a gene that is mutated in a subject having a genetic form of hearing loss or vestibular dysfunction, in one or more inner ear cells. The compositions described herein can be administered to a subject to treat or prevent hearing loss (e.g., sensorineural hearing loss), tinnitus, and/or vestibular dysfunction (e.g., vertigo, dizziness, imbalance, bilateral vestibulopathy, oscillopsia, or a balance disorder).

In a first aspect, the present disclosure provides a method of reducing nucleic acid vector-induced toxicity in the inner ear of a subject, the method including administering to the subject an effective amount of (a) a nucleic acid vector including a ubiquitous promoter operably linked to a polynucleotide encoding a therapeutic agent; (b) and an inhibitor of inflammatory or immune signaling, in which the nucleic acid vector is locally administered to the middle or inner ear.

In another aspect, the present disclosure provides a method of reducing off-target transduction of immune cells in the inner ear, the method including administering to (e.g., contacting) a mixed population of inner ear cells and immune cells an effective amount of a composition including a nucleic acid vector including a ubiquitous promoter operably linked to a polynucleotide encoding a therapeutic agent in combination with an inhibitor of inflammatory or immune signaling.

In another aspect, the present disclosure provides a method of improving therapeutic efficacy of a nucleic acid vector in an inner ear of a subject, the method including administering to the subject an effective amount of (a) a nucleic acid vector including a ubiquitous promoter operably linked to a polynucleotide encoding a therapeutic agent; (b) and an inhibitor of inflammatory or immune signaling, in which the nucleic acid vector is locally administered to the middle or inner ear.

In another aspect, the present disclosure provides a method of treating an inner ear dysfunction in a subject in need thereof, the method including administering to the subject an effective amount of (a) a nucleic acid vector including a ubiquitous promoter operably linked to a polynucleotide encoding a therapeutic agent; (b) and an inhibitor of inflammatory or immune signaling, in which the nucleic acid vector is locally administered to the middle or inner ear.

In another aspect, the present disclosure provides a method of reducing immune cell number and/or activity in an inner ear of a subject, the method including administering to the subject an effective amount of (a) a nucleic acid vector including a ubiquitous promoter operably linked to a polynucleotide encoding a therapeutic agent; (b) and an inhibitor of inflammatory or immune signaling, in which the nucleic acid vector is locally administered to the middle or inner ear.

In some embodiments of any of the foregoing aspects, the nucleic acid vector is a viral vector. In some embodiments, the viral vector is an adeno-associated viral (AAV) vector. In some embodiments, the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S capsid.

In some embodiments of any of the foregoing aspects, the ubiquitous promoter is an H1 promoter, a 7SK promoter, an apolipoprotein E-human α1-antitrypsin promoter (hAAT), a CK8 promoter, a murine U1 promoter (mU1a), an elongation factor 1α (EF-1α) promoter, an early growth response 1 (EGR1) promoter, a thyroxine binding globulin (TBG) promoter, a phosphoglycerate kinase (PGK) promoter, a CAG promoter, a chicken β-actin (CBA) promoter, an smCBA promoter, a CB7 promoter, a hybrid CMV enhancer/human β-actin promoter, a human β-actin promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), a CASI promoter, a dihydrofolate reductase (DHFR) promoter, a murine mammary tumor virus LTR promoter, an adenovirus major late (Ad MLP) promoter, a β-globin promoter (e.g., a minimal β-globin promoter), an HSV promoter (e.g., a minimal HSV ICPO promoter or a truncated HSV ICPO promoter), an SV40 promoter (e.g., an SV40 minimal promoter or an SV40 early promoter), a rous sarcoma virus (RSV) promoter, an eukaryotic translation initiation factor 4A1 (EIF4A1) promoter, a ferritin heavy (FerH) promoter, a ferritin light (FerL) promoter, a glyceraldehyde-3-phospohate dehydrogenase (GAPDH) promoter, a heat shock protein family A member 5 (HSPA5) gene, a heat shock protein family A member 4 (HSPA4) promoter, a ubiquitin B (UBB) promoter, or a U6 promoter.

In some embodiments of any of the foregoing aspects, the therapeutic agent is an inner ear protein, a peptide, an antibody or antigen-binding fragment thereof, an inhibitory nucleic acid, a microRNA (miRNA), or a component of a gene editing system (e.g., a nuclease or a guide RNA (gRNA)). In some embodiments, the inner ear protein is a protein that is natively (i.e., endogenously) expressed by inner ear cells. In some embodiments, the inhibitory nucleic acid is a short hairpin RNA (shRNA), a microRNA-adapted shRNA (shmiRNA), or an antisense oligonucleotide (ASO). In some embodiments, the component of a gene editing system is a nuclease. In some embodiments, the nuclease is a zinc finger nuclease (ZFN), transcription activator-like effector nuclease (TALEN), or clustered regularly interspaced short palindromic repeats (CRISPR) nuclease. In some embodiments, the CRISPR nuclease is a CRISPR-Cas9 or CRISPR-Cas12 nuclease, in which the CRISPR nuclease further includes a guide RNA (gRNA) sequence.

In some embodiments of any of the foregoing aspects, the inhibitor of inflammatory or immune signaling is an anti-inflammatory agent, an inhibitor of cell-mediated immunity, or a cellular de-targeting agent. In some embodiments, the anti-inflammatory agent and/or the inhibitor of cell-mediated immunity is a small molecule, peptide, antibody or antigen-binding fragment thereof, inhibitory nucleic acid (e.g., siRNA, shRNA, shmiRNA, or ASO), or a nuclease (e.g., a ZFN, TALEN, or CRISPR nuclease).

In some embodiments of any of the foregoing aspects, the anti-inflammatory agent or inhibitor of cell-mediated immunity is a corticosteroid (e.g., prednisone, prednisolone, methylprednisolone, betamethasone, dexamethasone, hydrocortisone, or deflazacort), a nonsteroidal anti-inflammatory drug (NSAID) (e.g., celecoxib, diclofenac, diflunisal, etodolac, indomethacin, ketoprofen, ketorolac, nabumetone, aspirin, ibuprofen, ketoprofen, naproxen, oxaprozin, piroxicam, salsalate, sulindac, tolmetin), methotrexate, hydroxychloroquine, sulfasalazine, leflunomide, cyclophosphamide, azathioprine, 6-mercaptopurine, 6-thioguanine, 5-aminoalicylic acid, mesalamine, balsalazide, olsalazine, an antibiotic, an anti-histamine, thalidomide, lenalidomide, pomalidomide, pentoxifylline, bupropion, a 5-HT2A receptor agonist, BX795, MRT68844, MRT67307, TPCA-1, Cyt387, AZD1480, ruxolitinib, tofacitinib, zinc, rocaglamide, mesopram, GIT27, StA-IFN-1, StA-IFN-2, StA-IFN-4, StA-IFN-5, Atgam®, thymoglobulin, azathioprine, a calcineurin inhibitor, cromolyn, cyclosporin A, myriocin, dimethyl fumarate, fingolimod, tofacitinib, fumaric acid ester, glatiramer acetate, hydroxyurea, IFNγ, IFNβ, IL-11, leflunomide, leukotriene receptor antagonist, long-acting beta2 agonist, mitoxantrone, mycophenolate mofetil, retinoid, salicylic acid, short-acting beta2 agonist, sirolimus, everolimus, zotarolimus, teriflunomide, theophylline, adalimumab, abatacept, anakinra, certolizumab, etanercept, golimumab, infliximab, rituximab, or tocilizumab abatacept, adalimumab, alemtuzumab, infliximab, adalimumab, certolizumab pegol, natalizumab, ruxolitinib, tofacitinib, muromonab, anti-IL-2, belimumab, golimumab, infliximab, basiliximab, daclizumab, natalizumab, ocrelizumab, rituximab, tocilizumab, ustekinumab, milatuzumab, DRα1-MOG-35-35, RTL1000, C36L1, vedolizumab, baricitinib, upadacitinib, or alefacept. In some embodiments, the anti-inflammatory agent or inhibitor of cell-mediated immunity is a corticosteroid, such as dexamethasone.

In some embodiments of any of the foregoing aspects, the inhibitor of cell-mediated immunity is an inhibitory nucleic acid or a nuclease including a gRNA having a sequence that is complementary to a sequence of a gene selected from the group including TNF receptor superfamily member 1A or 1B (TNFRSF1 A/B), TNF receptor superfamily member 13A or 13B (TNFRSF13 A/B), C-C motif chemokine ligand 8 (CCL8), bone marrow stromal cell antigen 2 (BST2), beta-2-microglobulin (B2M), histocompatibility 2, Q region locus 6 (H2-Q6), histocompatibility 2, T region locus 23 (H2-T23), proteasome 20S subunit beta 9 (PSMB9), integral membrane protein 2B (ITM2B), histocompatibility 2, class II, locus MB1 (H2-DMB1), small secreted protein interferon-induced (AW112010), histocompatibility 2, K region locus 1 (H2-K1), histocompatibility 2, D region locus 1 (H2-D1), CD74 molecule (CD74), histocompatibility 2, class II antigen A (H2-AA), BPI fold containing family A member 1 (BPIFA1), histocompatibility 2, class II antigen A, beta 1 (H2-AB1), CD86 molecule (CD86), and C-X-C motif chemokine receptor 3 (CXCR3).

In some embodiments of any of the foregoing aspects, the cellular de-targeting agent is nucleic acid sequence targeted by a microRNA expressed in an immune cell. In some embodiments, a polynucleotide encoding the nucleic acid sequence targeted by a microRNA expressed in an immune cell is included in the nucleic acid vector encoding the therapeutic agent. In some embodiments, the microRNA expressed in an immune cell is miR-9, miR-15a/16, miR-21, miR-23a, miR-24, miR-29a, let-7, miR-98, miR-106a, miR-125a˜99b˜let-7e cluster, miR-125b, miR-126, miR-127, miR-142, miR-145, miR-146a/b, miR-147b, miR-150, miR-155, miR-181, miR-187, miR-212, miR-222, miR-223, miR-451, miR-511, miR-720, miR-886-5p, and miR-4661, hsa-miR-378_st, hsa-miR-31_st, hsa-miR-935_st, hsa-miR-143_st, hsa-miR-362-5p_st. hsa-miR-532-5p_st, hsa-miR-500-star_st, hsa-miR-663_st, hsa-miR-125a-5p_st, hsa-miR-150_st, HBII-239_st, HBII-429_st, HBII-202_st, U27_st, U95_st, hsa-miR-768-5p_st, hsa-miR-223_st, or hsa-miR-652_st.

In some embodiments of any of the foregoing aspects, the cellular de-targeting agent is an inhibitory nucleic acid having complementarity to a transduction-permissive gene, or a nuclease including a gRNA having complementarity to a transduction permissive gene, or a polynucleotide encoding the same. In some embodiments, the polynucleotide encoding the inhibitory nucleic acid or the nuclease is operably linked to an immune-cell specific promoter. In some embodiments, the immune cell-specific promoter is a macrophage-specific promoter. In some embodiments, the macrophage-specific promoter is a colony stimulating factor 1 (CSF-1) promoter, colony stimulating factor 1 receptor (CSF1R) promoter, CD68 molecule (CD68) promoter, CD4 molecule (CD4) promoter, CD2 molecule (CD2) promoter, C-X3-C motif chemokine receptor 1 (CX3CR1) promoter, microfibril associated protein 4 (MFAP4) promoter, macrophage scavenger receptor 1 (MSR1) promoter, integrin subunit alpha M (ITGAM) promoter, integrin subunit alpha X (ITGAX) promoter, CD207 molecule (CD207) promoter, adhesion G protein-coupled receptor E1 (ADGRE1) promoter, or SP146-C1 promoter. In some embodiments, the immune cell-specific promoter is a T cell-specific promoter. In some embodiments, the T cell-specific promoter is a CD4 molecule (CD4) promoter, CD8 molecule (CD8) promoter, CD69 molecule (CD69) promoter, tumor necrosis factor alpha (TNFα) promoter, interleukin 2 (IL-2) promoter, C-X-C- motif chemokine receptor 3 (CXCR3) promoter, T-Box transcription factor 21 (TBX21) promoter, interleukin 4 (IL-4) promoter, interleukin 5 (IL-5) promoter, interleukin 9 (IL-9) promoter, interleukin 10 (IL-10) promoter, interleukin 17 (IL-17) promoter, C-C motif chemokine receptor 4 (CCR4) promoter, C-C motif chemokine receptor 6 (CCR6), GATA binding protein 3 (GATA3) promoter, interferon regulatory factor 4 (IRF4) promoter, killer cell lectin like receptor B1 (KLRB1), RAR related orphan receptor C (RORC) promoter, Fc fragment of IgG receptor IIIa (FCGR3A), T cell beta-chain (TCR3) promoter, CIFT promoter, or human CD3delta promoter.

In some embodiments of any of the foregoing aspects, the inhibitory nucleic acid inhibitor of inflammatory or immune signaling is a naked nucleic acid.

In some embodiments of any of the foregoing aspects, the inhibitory nucleic acid or the nuclease inhibitor of inflammatory or immune signaling is encoded in the nucleic acid vector including the ubiquitous promoter (e.g., operably linked to the ubiquitous promoter, such as in a polycistronic vector, or operably linked to a different promoter). In some embodiments, the inhibitory nucleic acid or the nuclease inhibitor of inflammatory or immune signaling is encoded in a second nucleic acid vector, in which the polynucleotide encoding the inhibitory nucleic acid or nuclease is operably linked to an immune cell-specific promoter.

In some embodiments of any of the foregoing aspects, the nucleic acid vector-induced toxicity is inflammation-induced toxicity or cell-mediated immunity-induced toxicity. In some embodiments, reducing nucleic acid vector-induced toxicity includes reducing nucleic acid vector-induced toxicity by 5%, 10%, 15%, 20%, 25%, 30%, 35%, 40%, 45%, 50%, 55%, 60%, 65%, 70%, 75%, 80%, 85%, 90%, 95%, 99% or more as compared to nucleic acid vector-induced toxicity in a subject administered the nucleic acid vector in the absence of an inhibitor of inflammatory or immune signaling.

In some embodiments of any of the foregoing aspects, the mixed population of inner ear cells includes inner ear hair cells, inner ear supporting cells, spiral ganglion neurons, Scarpa's ganglion neurons, Claudius cells, spiral prominence cells, root cells, interdental cells, basal cells of the stria vascularis, intermediate cells of the stria vascularis, marginal cells of the stria vascularis, endothelial cells of cochlear capillaries, cochlear fibrocytes, cells of Reissner's membrane, and cochlear glial cells. In some embodiments, the off-target transduction of inner ear immune cells is reduced by 5%, 10%, 15%, 20%, 25%, 30%, 35%, 40%, 45%, 50%, 55%, 60%, 65%, 70%, 75%, 80%, 85%, 90%, 95%, 99% or more as compared to a mixed population of inner ear cells and immune cells treated with the nucleic acid vector in the absence of an inhibitor of inflammatory or immune signaling.

In some embodiments of any of the foregoing aspects, improving therapeutic efficacy includes preventing or reducing hearing loss, preventing or reducing tinnitus, delaying development of hearing loss, slowing the progression of hearing loss, improving hearing, improving balance, reducing dizziness, reducing vertigo, delaying development of vestibular dysfunction, slowing the progression of vestibular dysfunction, increasing expression and/or activity of the therapeutic agent in one or more inner ear cells, increasing inner ear hair cell development, increasing inner ear hair cell numbers, increasing or inducing inner ear hair cell maturation, increasing inner ear hair cell survival, increasing inner ear hair cell regeneration, improving inner ear hair cell function, improving inner ear supporting cell function, improving inner ear supporting cell proliferation, improving inner ear supporting cell maturation, increasing inner ear supporting cell numbers, or increasing inner ear supporting cell survival.

In some embodiments of any of the foregoing aspects, the inner ear hair cells are cochlear inner hair cells (IHC), cochlear outer hair cells (OHC), type I vestibular hair cells, or type II vestibular hair cells.

In some embodiments of any of the foregoing aspects, the inner ear supporting cells are cochlear supporting cells or vestibular supporting cells. In some embodiments, the cochlear supporting cells include Border cells, inner phalangeal cells, inner pillar cells, outer pillar cells, first row Deiters' cells, second row Deiters' cells, third row Deiters' cells, and/or Hensen's cells.

In some embodiments of any of the foregoing aspects, the inner ear dysfunction is hearing loss. In some embodiments, the hearing loss is genetic hearing loss. In some embodiments, the genetic hearing loss is autosomal dominant hearing loss, autosomal recessive hearing loss, or X-linked hearing loss. In some embodiments, the hearing loss is acquired hearing loss. In some embodiments, the acquired hearing loss is noise-induced hearing loss, age-related hearing loss, disease or infection-related hearing loss, head trauma-related hearing loss, or ototoxic drug-induced hearing loss.

In some embodiments of any of the foregoing aspects, the inner ear dysfunction is tinnitus.

In some embodiments of any of the foregoing aspects, the inner ear dysfunction is vestibular dysfunction. In some embodiments, the vestibular dysfunction is vertigo, dizziness, loss of balance (imbalance), bilateral vestibulopathy, oscillopsia, or a balance disorder. In some embodiments, the vestibular dysfunction is loss of balance. In some embodiments, the vestibular dysfunction is vertigo. In some embodiments, the vestibular dysfunction is dizziness. In some embodiments, the vestibular dysfunction is bilateral vestibulopathy. In some embodiments, the vestibular dysfunction is oscillopsia. In some embodiments, the vestibular dysfunction is a balance disorder. In some embodiments, the vestibular dysfunction is age-related vestibular dysfunction, head trauma-related vestibular dysfunction, disease or infection-related vestibular dysfunction, or ototoxic drug-induced vestibular dysfunction. In some embodiments, the vestibular dysfunction is associated with a genetic mutation. In some embodiments, the vestibular dysfunction is idiopathic vestibular dysfunction.

In some embodiments of any of the foregoing aspects, the ototoxic drug is an aminoglycoside, an antineoplastic drug, ethacrynic acid, furosemide, a salicylate, or quinine.

In some embodiments of any of the foregoing aspects, the immune cells are monocytes and/or dendritic cells. In some embodiments, reducing a number of immune cells in the inner ear of a subject includes reducing the number of immune cells in the inner ear by 5%, 10%, 15%, 20%, 25%, 30%, 35%, 40%, 45%, 50%, 55%, 60%, 65%, 70%, 75%, 80%, 85%, 90%, 95%, 99% or more as compared to the number of inner ear immune cells in a subject treated with the nucleic acid vector in the absence of an inhibitor of inflammatory or immune signaling. In some embodiments, reducing a number and/or activity of immune cells includes reducing immune cell recruitment to the inner ear, increasing immune cell death in the inner ear, reducing immune cell migration in the inner ear, reducing activation of immune cells, reducing phagocytosis by immune cells, reducing antibody-dependent cellular cytotoxicity by immune cells, reducing immune cell polarization, reducing immune cell proliferation, reducing immune cell differentiation, reducing immune cell cytokine production (e.g., reducing pro-inflammatory cytokine production), reducing immune cell antigen presentation, reducing immune cell maturation, or reducing immune cell degranulation.

In some embodiments of any of the foregoing aspects, the improved therapeutic efficacy is improved durability and/or improved magnitude of a response in a subject. In some embodiments, the response is hearing recovery. In some embodiments, the response is reduction or elimination of tinnitus. In some embodiments, the response is recovery of vestibular function (e.g., improvement or restoration of balance).

In some embodiments of any of the foregoing aspects, the administering includes simultaneous administration of the nucleic acid vector and the inhibitor of inflammatory or immune signaling to the subject or to the mixed population of inner ear cells and immune cells. In some embodiments, the simultaneous administration includes simultaneous local administration of the nucleic acid vector encoding the therapeutic agent and local administration of the inhibitor of inflammatory or immune signaling. In some embodiments, the simultaneous administration includes simultaneous local administration of the nucleic acid vector encoding the therapeutic agent and systemic administration of the inhibitor of inflammatory or immune signaling.

In some embodiments of any of the foregoing aspects, the administering includes sequential administration of the nucleic acid vector encoding the therapeutic agent and the inhibitor of inflammatory or immune signaling, in which the sequential administration includes: a) administration of the nucleic acid vector encoding the therapeutic agent prior to administration of the inhibitor of inflammatory or immune signaling; or b) administration of the nucleic acid vector encoding the therapeutic agent following administration of the inhibitor of inflammatory or immune signaling. In some embodiments, the sequential administration includes local administration of the nucleic acid vector and the inhibitor of inflammatory or immune signaling. In some embodiments, the sequential administration includes local administration of the nucleic acid vector encoding the therapeutic agent and systemic administration of the inhibitor of inflammatory or immune signaling.

In some embodiments of any of the foregoing aspects, local administration includes local administration to the middle or inner ear. In some embodiments, local administration to the middle or inner ear includes administration to a semicircular canal, transtympanic administration, intratympanic administration, administration into the perilymph, administration into the endolymph, administration to or through the round window, or administration to or through the oval window.

In some embodiments of any of the foregoing aspects, systemic administration includes intravenous, intramuscular, subcutaneous, intraperitoneal, transmucosal, or oral administration.

In some embodiments of any of the foregoing aspects, the subject is a human.

### BRIEF DESCRIPTION OF THE DRAWINGS

The file of this patent or application contains at least one drawing executed in color. Copies of this patent or patent application publication with color drawings will be provided by the Patent and Trademark Office upon request and payment of the necessary fee.

FIGS. 1A-1F are a series of boxplots and individual sample points showing log 10(transcripts per million; TPM) of exemplary significantly differentially expressed apoptosis genes Fas cell surface death receptor (Fas; FIG. 1A), RELA proto-oncogene NF-κB subunit (Rela; FIG. 1B), and tumor necrosis factor (TNF; FIG. 1C) and allograft rejection signature genes CD74 molecule (CD74; FIG. 1D), CD86 molecule (CD86; FIG. 1E), and C-X-C motif chemokine receptor 3 (CXCR3; FIG. 1F), as measured by bulk RNA-sequencing (RNA-seq).

FIGS. 2A-2C are a series of uniform manifold approximation and projection (UMAP) plots showing single cell RNA-seq profiling of murine cochlea injected with an adeno-associated viral (AAV) vector containing the ubiquitous small chicken beta actin (smCBA) promoter (AAV-smCBA) or an inner ear sensory cell-specific myosin 15 (Myo15) promoter (AAV-Myo15). UMAP plot showing filtered and clustered single cells colored by sample of origin (FIG. 2A). Same UMAP as in FIG. 2A, with cells colored by whether they are immune or non-immune (other) cells (FIG. 2B). UMAP showing clustered immune cells only, colored by cell type (FIG. 2C).

FIGS. 3A-3I are a series of bar plots showing differences in immune cell numbers between AAV-smCBA- and AAV-Myo15-treated samples. Percent (%) of immune cells in each sample are shown by immune cell types, which include B cells (FIG. 3A), cytotoxic T cells (FIG. 3B), dendritic cells (FIG. 3C), erythroid cells (FIG. 3D), granulocytes (FIG. 3E), macrophages (FIG. 3F), monocytes (FIG. 3G), neutrophils (FIG. 3H), and natural killer (NK)/natural killer T (NKT) cells (FIG. 3I). These results demonstrate an increase in the numbers of dendritic cells and monocytes in in AAV-smCBA-treated samples as compared to AAV-Myo15-treated samples.

FIGS. 4A-4D are a series of violin plots showing human otoferlin (hOTOF) mRNA expression in different cell types, including macrophages (FIG. 4A), neutrophils (FIG. 4B), spiral ganglion type I neurons (SGN Type I; FIG. 4C), and cochlear supporting cells (FIG. 4D), as measured by RNA-seq. Off-target expression in immune cells was increased in AAV-smCBA-treated samples as compared to AAV-Myo15-treated samples.

FIG. 5 is a volcano plot showing results of differential expression testing between macrophage cells from AAV-smCBA and AAV-Myo15 treated cochleae. Significantly up- and down-regulated genes are displayed in blue and pink, respectively. Top up-regulated genes, many related to antigen presentation, are labeled in pink.

FIGS. 6A-6C are a series of plots showing ligand-receptor interaction analysis. Total number of significant ligand-receptor interactions identified in each sample (FIG. 6A). Heatmap showing receptor-ligand interactions involving Tnfsfl3 (rows) per sample. Cells are colored according to whether each interaction was found to be significant. Box highlights a set of interactions uniquely significant in smCBA samples (FIG. 6B). Dot plot showing pairs of cell types involved in Tnfsf13 ligand-receptor interactions. Dot color and size indicate number of AAV-smCBA, minus number of AAV-Myo15, samples with one or more significant interactions between each given pair of cell types. Cells are left blank if no significant interactions were identified in any of the samples (FIG. 6C).

### DEFINITIONS

As used herein, the term “about” refers to a value that is within 10% above or below the value being described.

As used herein, “administration” refers to providing or giving a subject a therapeutic agent (e.g., a nucleic acid vector described herein), by any effective route. Exemplary routes of administration are described herein below.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the terms “effective amount,” “therapeutically effective amount,” and a “sufficient amount” of a composition, vector construct, or viral vector described herein refer to a quantity sufficient to, when administered to the subject, including a mammal, for example a human, effect beneficial or desired results, including clinical results, and, as such, an “effective amount” or synonym thereto depends upon the context in which it is being applied. For example, in the context of treating an inner ear dysfunction (e.g., hearing loss, tinnitus, or vestibular dysfunction), it is an amount of the composition, vector construct, or viral vector sufficient to achieve a treatment response as compared to the response obtained without administration of the composition, vector construct, or viral vector. The amount of a given composition described herein that will correspond to such an amount will vary depending upon various factors, such as the given agent, the pharmaceutical formulation, the route of administration, the type of disease or disorder, the identity of the subject (e.g. age, sex, weight) or host being treated, and the like, but can nevertheless be routinely determined by one skilled in the art. Also, as used herein, a “therapeutically effective amount” of a composition, vector construct, or viral vector of the present disclosure is an amount which results in a beneficial or desired result in a subject as compared to a control. As defined herein, a therapeutically effective amount of a composition, vector construct, or viral vector of the present disclosure may be readily determined by one of ordinary skill by routine methods known in the art. Dosage regimen may be adjusted to provide the optimum therapeutic response.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., an inner ear cell).

As used herein, the term “enhancer” refers to a type of regulatory element that can increase the efficiency of transcription regardless of the distance or orientation of the enhancer relative to the transcription start site. Accordingly, enhancers can be placed upstream or downstream of the transcription start site or at a considerable distance from the promoter. Enhancers may also overlap physically and functionally with promoters. A number of polynucleotides comprising promoter sequences (e.g., ubiquitous promoter sequences) also contain enhancer sequences.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell, e.g., a human inner ear cell). Exogenous materials include those that are provided from an external source to an organism or to cultured matter extracted there from.

As used herein, the term “exon” refers to a region within the coding region of a gene, the nucleotide sequence of which determines the amino acid sequence of the corresponding protein. The term exon also refers to the corresponding region of the RNA transcribed from a gene. Exons are transcribed into pre-mRNA and may be included in the mature mRNA depending on the alternative splicing of the gene. Exons that are included in the mature mRNA following processing are translated into protein, wherein the sequence of the exon determines the amino acid composition of the protein.

As used herein, the term “heterologous” refers to a combination of elements that is not naturally occurring. For example, a heterologous transgene refers to a transgene that is not naturally expressed by the promoter to which it is operably linked.

As used herein, the terms “increasing” and “decreasing” refer to modulating resulting in, respectively, greater or lesser amounts, of function, expression, or activity of a metric relative to a reference. For example, subsequent to administration of a composition in a method described herein, the amount of a marker of a metric (e.g., transgene expression) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the phrase “inhibitor of inflammatory or immune cell signaling” refers to an agent (e.g., small molecule, peptide, antibody or antigen-binding fragment thereof, or nucleic acid or nucleic acid vector containing the same) that is capable of inhibiting inflammation or cell-mediated immunity. For example, an inhibitor of inflammatory or immune cell signaling, when administered in an effective amount (e.g., a therapeutically effective amount) to cells of the inner ear in vitro or in vivo, is capable of reducing immune cell recruitment, increasing immune cell death, reducing immune cell migration, reducing activation of immune cells, reducing phagocytosis by immune cells, reducing antibody-dependent cellular cytotoxicity by immune cells, reducing immune cell polarization toward a Type 1 phenotype, reducing immune cell proliferation, reducing immune cell differentiation, reducing immune cell pro-inflammatory cytokine production, reducing immune cell antigen presentation, reducing immune cell maturation, or reducing immune cell degranulation. Exemplary inhibitors of inflammatory or immune cell signaling are described herein.

As used herein, the term “inner ear cell type” refers to a cell type found in the inner ear (e.g., cochlea and/or vestibular system) of a subject (e.g., a human subject). Inner ear cell types include inner hair cells, outer hair cells, vestibular hair cells, vestibular dark cells, vestibular fibrocytes, Scarpa's ganglion neurons (vestibular ganglion neurons), endothelial cells of vestibular capillaries, vestibular supporting cells, Border cells, inner phalangeal cells, inner pillar cells, outer pillar cells, first row Deiters' cells, second row Deiters' cells, third row Deiters' cells, Hensen's cells, Claudius cells, spiral prominence cells, root cells, interdental cells, basal cells of the stria vascularis, intermediate cells of the stria vascularis, marginal cells of the stria vascularis, spiral ganglion neurons, endothelial cells of cochlear capillaries, fibrocytes, cells of Reissner's membrane, and glial cells.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration, administration to the middle or inner ear, and administration to a mucous membrane of the subject, wherein the administration is intended to have a local and not a systemic effect.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, the term “polynucleotide” refers to a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “promoter” refers to a recognition site on DNA that is bound by an RNA polymerase. The polymerase drives transcription of the transgene. A “ubiquitous promoter” is a promoter capable of robustly driving gene expression in a broad range of cells, tissues, and cell types. Exemplary ubiquitous promoters are described herein.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “sample” refers to a specimen (e.g., blood, blood component (e.g., serum or plasma), urine, saliva, amniotic fluid, cerebrospinal fluid, tissue (e.g., neural tissue, placental tissue, or dermal tissue), pancreatic fluid, chorionic villus sample, and cells (e.g., inner ear cells or stem cells)) isolated from a subject.

As used herein, the term “simultaneous administration” refers to administration of two or more therapeutic agents described herein (e.g., a nucleic acid vector containing a ubiquitous promoter operably linked to a transgene and an inhibitor of inflammatory or immune signaling) to a subject (e.g., a human) or a population of cells (e.g., a mixed population of inner ear cells and immune cells), in which the first therapeutic agent is administered within a time period of 15 minutes, 10 minutes, 5 minutes, 2 minutes or less relative to the administration of the second agent and vice-versa.

As used herein, the term “sequential administration” refers to administration of two or more therapeutic agents described herein (e.g., a nucleic acid vector containing a ubiquitous promoter operably linked to a transgene and an inhibitor of inflammatory or immune signaling) to a subject (e.g., a human) or a population of cells (e.g., a mixed population of inner ear cells and immune cells), in which the first therapeutic agent is administered from 20 minutes up to 1 hour, from 20 minutes up to 2 hours, from 20 minutes up to 3 hours, from 20 minutes up to 4 hours, from 20 minutes up to 5 hours, from 20 minutes up to 6 hours, from 20 minutes up to 7 hours, from 20 minutes up to 8 hours, from 20 minutes up to 9 hours, from 20 minutes up to 10 hours, from 20 minutes up to 11 hours, from 20 minutes up to 12 hours, from 20 minutes up to 13 hours, 14 hours, from 20 minutes up to 15 hours, from 20 minutes up to 16 hours, from 20 minutes up to 17 hours, from 20 minutes up 18 hours, from 20 minutes up to 19 hours, from 20 minutes up to 20 hours, from 20 minutes up to 21 hours, from 20 minutes up to 22 hours, from 20 minutes up to 23 hours, from 20 minutes up to 24 hours, or from 20 minutes up to 1-7, 1-14, 1-21 or 1-30 days before or after the second therapeutic agent and vice-versa.

As used herein, the term “transcription regulatory element” refers to a nucleic acid that controls, at least in part, the transcription of a gene of interest. Transcription regulatory elements may include promoters, enhancers, and other nucleic acids (e.g., polyadenylation signals) that control or help to control gene transcription. Examples of transcription regulatory elements are described, for example, in Lorence, Recombinant Gene Expression: Reviews and Protocols (Humana Press, New York, NY, 2012).

As used herein, the term “transfection” refers to any of a wide variety of techniques commonly used for the introduction of exogenous DNA into a prokaryotic or eukaryotic host cell, e.g., electroporation, lipofection, calcium phosphate precipitation, DEAE-dextran transfection, Nucleofection, squeeze-poration, sonoporation, optical transfection, magnetofection, impalefection and the like.

As used herein, the terms “subject” and “patient” refer to a mammal (e.g., such as, e.g., a human). A subject to be treated according to the methods described herein may be one who has been diagnosed with hearing loss (e.g., sensorineural hearing loss, auditory neuropathy, or deafness), tinnitus, or vestibular dysfunction (e.g., dizziness, vertigo, balance loss, bilateral vestibulopathy, oscillopsia, or a balance disorder) or one at risk of developing these conditions (e.g., a subject at risk of developing hearing loss, tinnitus, or vestibular dysfunction due to age, head trauma, acoustic trauma (e.g., exposure to loud noise), disease or infection, treatment with ototoxic drugs, a genetic mutation, or a family history of hearing loss, tinnitus, or vestibular dysfunction). Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the terms “transduction” and “transduce” refer to a method of introducing a vector construct or a part thereof into a cell. Wherein the vector construct is contained in a viral vector such as for example an AAV vector, transduction refers to viral infection of the cell and subsequent transfer and integration of the vector construct or part thereof into the cell genome.

As used herein, the term “transduction-permissive gene” refers to any mammalian gene that is required for or that facilitates effective transduction of a viral vector (e.g., an AAV vector) into a mammalian cell. For example, a transduction-permissive gene may be any gene that is required for or that facilitates any one of the following steps of viral vector transduction: cellular uptake, post-entry trafficking, nuclear import, second-strand synthesis, and transgene expression. Inhibition of a transduction-permissive gene according to the methods disclosed herein may be a useful approach for blocking transduction of the viral vector into a particular cell type (e.g., an immune cell).

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “vector” includes a nucleic acid vector, e.g., a DNA vector, such as a plasmid, cosmid, or artificial chromosome, an RNA vector, a virus, or any other suitable replicon (e.g., viral vector). A variety of vectors have been developed for the delivery of polynucleotides encoding exogenous proteins into a prokaryotic or eukaryotic cell. Examples of such expression vectors are described in, e.g., Gellissen, Production of Recombinant Proteins: Novel Microbial and Eukaryotic Expression Systems (John Wiley & Sons, Marblehead, M A, 2006). Expression vectors suitable for use with the compositions and methods described herein contain a polynucleotide sequence as well as, e.g., additional sequence elements used for the expression of proteins and/or the integration of these polynucleotide sequences into the genome of a mammalian cell. Certain vectors that can be used for the expression of transgene as described herein include vectors that contain regulatory sequences, such as promoter and enhancer regions, which direct gene transcription. Other useful vectors for expression of a transgene contain polynucleotide sequences that enhance the rate of translation of the transgene or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein, the terms “wild-type” and “WT” refer to a genotype with the highest frequency for a particular gene in a given organism.

### DETAILED DESCRIPTION

Described herein are compositions and methods for transducing inner ear cells (e.g., cells of the cochlea and/or vestibular system, such as inner hair cells, outer hair cells, vestibular hair cells, cochlear supporting cells, and vestibular supporting cells) by administering a nucleic acid vector (e.g., an AAV vector) containing a polynucleotide encoding an therapeutic agent (e.g., a protein that is expressed in normal inner ear cells, a protein that regulates inner ear cell survival, regeneration, cell fate, and/or cell proliferation, an inhibitory RNA, an miRNA, or a component of a gene editing system) under the control of a ubiquitous promoter in combination with an agent that reduces nucleic acid vector-induced (e.g., AAV vector-induced) inflammation and/or immune response in the inner ear (i.e., an inhibitor of inflammatory or immune signaling). Such compositions are particularly useful for mitigating inflammatory or immune toxicity associated with off-target expression of a protein encoded by a transgene in a nucleic acid vector in inner ear immune cells, and can, therefore, be used to improve the therapeutic efficacy of nucleic acid vectors carrying polynucleotides targeted for ubiquitous expression (e.g., expression in two or more cell types) in the inner ear. The disclosed compositions can be administered to a subject to treat disorders associated with or caused by damage, degeneration, loss, and/or dysfunction of inner ear cells, such as hearing loss (e.g., sensorineural hearing loss), tinnitus, or vestibular dysfunction.

### Inner Ear Cells

The inner ear is populated by a number of specialized cells. Both the cochlea and vestibular system contain hair cells, which are the primary sensory cells of the inner ear. Cochlear hair cells are made up of two main cell types: inner hair cells (IHCs), which are responsible for sensing sound, and outer hair cells (OHCs), which are thought to amplify low-level sound. Vestibular hair cells are located in the semicircular canals and otolith organs (e.g., utricle and saccule) of the vestibular system, and are involved in the sensation of movement that contributes to the sense of balance and spatial orientation. Spiral ganglion neurons innervate cochlear hair cells and send axons into the central nervous system, while neurons of the vestibular ganglion innervate vestibular hair cells. Non-sensory cells called supporting cells reside between hair cells in the cochlea and in the vestibular system and perform a number of important functions, such as providing a structural scaffold to allow for mechanical stimulation of hair cells, maintaining the ionic composition of the endolymph and perilymph, and regulating synaptogenesis of ribbon synapses. Within the cochlea, supporting cells can be subdivided into five different types: 1) Hensen's cells, 2) Deiters' cells, 3) pillar cells; 4) inner phalangeal cells; and 5) border cells, all of which have distinct morphologies and patterns of gene expression. Mutations in genes expressed in cochlear hair cells, cochlear supporting cells, and/or spiral ganglion neurons have been associated with hearing loss (e.g., sensorineural hearing loss), auditory neuropathy, deafness, and tinnitus, as has damage, injury, degeneration, or loss (e.g., death) of these cells. Similarly, mutations in genes expressed in cells of the vestibular system (e.g., in vestibular hair cells, vestibular supporting cells, and/or vestibular ganglion neurons) and damage, injury, degeneration, or loss (e.g., death) of cells of the vestibular system have been associated with vestibular dysfunction (e.g., vertigo, dizziness, and/or balance loss). Gene therapy has recently emerged as an attractive therapeutic approach for treating hearing loss and vestibular dysfunction; however, given the large number of cell types that may need to be targeted to address the various causes of hearing loss and vestibular dysfunction, there exists a need to safely transduce the many cell types of the inner ear.

The present invention is based, in part, on the inventors' discovery that administration of an AAV vector containing a polynucleotide under regulatory control of a ubiquitous promoter to cells of the inner ear induced immune toxicity associated with increased expression of apoptosis genes in cells of the inner ear, off-target expression of the transgene in inner ear immune cells, activation of antigen-directed immunity mediated by major histocompatibility complex (MHC) genes, T cell genes, and macrophage genes, increases in the number of immune cells (e.g., monocytes and dendritic cells) in the inner ear, and elevated ligand-receptor interactions involving apoptosis pathways, such as, e.g., tumor necrosis family receptor super family (TNFRSF)-mediated interactions involving neutrophils and granulocytes. While systemic delivery of AAV vectors containing ubiquitous promoters was known to cause inflammation, it was not previously recognized that local administration of an AAV vector containing a ubiquitous promoter to the inner ear would have a similar effect. Without wishing to be bound by theory, these findings delineate a mechanistic underpinning to the toxicity associated with the use of ubiquitous promoters in gene therapy and provide a therapeutic avenue to mitigate the resulting toxicity by targeting immune mediators of nucleic acid vector-induced immune toxicity in the inner ear. The compositions and methods described herein can, therefore, be used to induce expression of a polynucleotide in cells of the inner ear while also mitigating toxicity resulting from immune activation. Thus, the methods and compositions described herein can be administered to a subject to treat a disorder caused by a genetic mutation in or damage, degeneration, loss, and/or dysfunction of one or more cell types of the inner ear, or to treat a disorder caused by a genetic mutation in or damage, degeneration, loss, and/or dysfunction of one or a subset of inner ear cell types.

Accordingly, the present disclosure features compositions and methods for transducing multiple cell types of the inner ear using a nucleic acid vector (e.g., an AAV vector) containing a therapeutic agent (e.g., a polynucleotide encoding an inner ear protein, an RNAi sequence, an miRNA, an antibody or an antigen-binding fragment thereof, or a component of a gene editing system, such as a nuclease) under regulatory control of a ubiquitous promoter in combination with an inhibitor of inflammatory or immune signaling. The compositions disclosed herein can be administered to a subject, such as a human subject, to promote the expression of the therapeutic agent (e.g., a polynucleotide, such as a polynucleotide corresponding to a gene that promotes or improves inner ear cell function, regeneration, maintenance, development, proliferation, or survival, or a polynucleotide that corresponds to a wild-type version of a gene that is mutated in a subject having a genetic form of hearing loss or vestibular dysfunction) in one or more inner ear cells.

The compositions described herein can be administered to a subject to treat or prevent hearing loss (e.g., sensorineural hearing loss), tinnitus, and/or vestibular dysfunction (e.g., vertigo, dizziness, imbalance, bilateral vestibulopathy, oscillopsia, or a balance disorder). The compositions disclosed herein may be administered to the subject using various approaches, including: 1) simultaneously or sequentially administering to the subject by way of local administration to the inner ear a nucleic acid vector containing a therapeutic agent (e.g., a polynucleotide encoding an inner ear protein, an RNAi sequence, an miRNA, an antibody or an antigen-binding fragment thereof, or a component of a gene editing system, such as a nuclease) and an inhibitor of inflammatory or immune signaling (e.g., small molecule, peptide, antibody or antigen-binding fragment thereof, nuclease, RNAi agent, or a nucleic acid vector containing a polynucleotide encoding the inhibitor of inflammatory or immune signaling); 2) administering to the subject by way of local administration to the inner ear a nucleic acid vector containing a polycistronic construct containing the therapeutic agent and an inhibitor of inflammatory or immune signaling, wherein the therapeutic agent and the inhibitor are operably linked to a single ubiquitous promoter or wherein the therapeutic agent is operably linked to a ubiquitous promoter and the inhibitor is independently operably linked to a ubiquitous or cell-specific promoter (e.g., an immune cell-specific promoter); and 3) simultaneously or sequentially administering a nucleic acid vector containing the therapeutic agent by way of local administration to the inner ear and systemically administering the inhibitor of inflammatory or immune signaling (e.g., small molecule, peptide, antibody or antigen-binding fragment thereof, nuclease, RNAi agent, or a nucleic acid vector containing a polynucleotide encoding the inhibitor).

In some embodiments, the nucleic acid vector (e.g., an AAV vector) and an inhibitor of inflammatory or immune signaling are administered to the inner ear of a subject in an amount sufficient to transduce 2 or more (e.g., 3, 4, 5, 6, 7, 8, 9, 10, 11, 12, 13, 14, 15, 20, or more) inner ear cell types selected from the group including inner hair cells, outer hair cells, vestibular hair cells, vestibular dark cells, vestibular fibrocytes, Scarpa's ganglion neurons (vestibular ganglion neurons), endothelial cells of vestibular capillaries, vestibular supporting cells, Border cells, inner phalangeal cells, inner pillar cells, outer pillar cells, first row Deiters' cells, second row Deiters' cells, third row Deiters' cells, Hensen's cells, Claudius cells, spiral prominence cells, root cells, interdental cells, basal cells of the stria vascularis, intermediate cells of the stria vascularis, marginal cells of the stria vascularis, spiral ganglion neurons, endothelial cells of cochlear capillaries, fibrocytes, cells of Reissner's membrane, and glial cells. The pantropic transduction of inner ear cells by the vector can be used to induce expression of a polynucleotide throughout the inner ear (e.g., using an AAV vector in which the polynucleotide is operably linked to a ubiquitous promoter). The ability to transduce 2 or more (e.g., 3, 4, 5, 6, 7, 8, 9, 10, 11, 12, 13, 14, 15, or more) different inner ear cell types without inducing clinically undesirable immune toxicity is beneficial for therapeutic approaches in which it is desirable to express a polynucleotide in many or most cell types of the inner ear, for example, to deliver the wild-type version of a gene that is mutated in many or all inner ear cell types, or to produce a large quantity of a secreted protein that produces a therapeutic effect at high concentrations.

### Nucleic Acid Vectors for the Expression of Heterologous Polynucleotides

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a polynucleotide sequence that contains or encodes a therapeutic agent (e.g., a polynucleotide encoding an inner ear protein, an RNAi sequence, an miRNA, an antibody or an antigen-binding fragment thereof, or a component of a gene editing system, such as a nuclease), as well as, e.g., additional sequence elements used for the expression of these agents and/or the integration of these polynucleotide sequences into the genome of a mammalian cell. Certain vectors that can be used for the expression of heterologous genes include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

### AAV Vectors for Nucleic Acid Delivery

In some embodiments, nucleic acids of the compositions and methods described herein are incorporated into recombinant AAV (rAAV) vectors and/or virions in order to facilitate their introduction into a cell. rAAV vectors useful in the compositions and methods described herein are recombinant nucleic acid constructs that include (1) a heterologous sequence to be expressed (e.g., a polynucleotide encoding an inner ear protein, an RNAi sequence, a miRNA, an antibody or an antigen-binding fragment thereof, or a component of a gene editing system, such as a nuclease) and (2) viral sequences that facilitate stability and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part but retain functional flanking ITR sequences. The AAV ITRs may be of any serotype suitable for a particular application. For use in the methods and compositions described herein, the ITRs can be AAV2 ITRs. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The nucleic acids and vectors described herein can be incorporated into a rAAV virion in order to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, and PHP.S. For inner ear gene therapy, AAV1, AAV2, AAV6, AAV9, Anc80, Anc80L65, DJ/9, 7m8, and PHP.B may be particularly useful. Serotypes evolved for transduction of the retina may also be used in the methods and compositions described herein. The first and second nucleic acid vectors (e.g., AAV vectors) in the compositions and methods described herein may have the same serotype or different serotypes. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J. Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

In some embodiments, the use of AAV vectors for delivering a functional transgene requires the use of a dual vector system, in in which the first member of the dual vector system encodes an N-terminal portion of a protein and the second member encodes a C-terminal portion of a protein such that, upon administration of the dual vector system to a cell, the polynucleotide sequences contained within the two vectors can join to form a single sequence that results in the production of a full-length protein.

In some embodiments, two or more AAV vectors described herein (e.g., 2, 3, 4, or more AAV vectors) may be used to express a single polynucleotide, such as a polynucleotide having a coding sequence of 3 kb or longer (e.g., 3 kb, 3.5 kb, 4 kb, 4.5 kb, 5 kb, 5.5 kb, 6 kb, 6.5 kb, or longer). For example, two or more AAV vectors may be used to express a polynucleotide encoding Otoferlin, which has a coding sequence of approximately 6 kb. In embodiments in which two or more AAV vectors are used to express a single polynucleotide, the coding sequence of the polynucleotide is divided between the vectors such that the full-length coding sequence can be reconstituted in vivo. In some embodiments, a dual vector system including two AAV vectors can be used to express a single polynucleotide. A portion of the coding sequence of the polynucleotide (e.g., a polynucleotide having a coding sequence of 3 kb, 3.5 kb, 4 kb, 4.5 kb, 5 kb, 5.5 kb, 6 kb, 6.5 kb, or longer) can be contained within each AAV vector. Exemplary dual vector systems include fragmented dual vectors, overlapping dual vectors, trans-splicing dual vectors, and dual hybrid vectors. These systems are described in McClements and MacLaren, Yale J Biol Med. 90:611-623, 2017, the disclosure of which is incorporated herein by reference.

In some embodiments, the dual vector system is an AAV1 dual vector system. In some embodiments, the dual vector system is an AAV9 dual vector system.

In some embodiments, the nucleic acid vectors described herein are used to express two or more polynucleotides (e.g., a single vector contains polynucleotides encoding two different proteins of interest). In some embodiments, the two or more polynucleotides are expressed using a bicistronic or polycistronic expression cassette. In some embodiments, the polycistronic expression cassette includes an internal ribosomal entry site (IRES) positioned between the two or more polynucleotides (e.g., an IRES positioned between the polynucleotides encoding two different proteins of interest). In some embodiments, the polycistronic expression cassette includes a foot-and-mouth disease virus 2A (FMDV 2A) polynucleotide, or a similar 2A polynucleotide (e.g., equine rhinitis A virus (E2A), porcine teschovirus-1 (P2A) or Thosea asigna virus (T2A)), positioned between the two or more polynucleotides (e.g., an FMDV 2A polynucleotide positioned between each nucleic acid encoding a protein of interest).

### Regulatory Sequences

The polynucleotides and nucleic acid vectors encoding the same described herein may be required to be expressed at sufficiently high levels to elicit a therapeutic benefit. Accordingly, polynucleotide expression may be mediated by a promoter sequence capable of driving robust expression of the disclosed polynucleotides. According to the methods and compositions disclosed herein, the promoter may be a heterologous promoter. The term “heterologous promoter”, as used herein, refers to a promoter that is not found to be operatively linked to a given encoding sequence in nature. Useful heterologous control sequences generally include those derived from sequences encoding mammalian or viral genes.

For purposes of the present disclosure, both heterologous promoters and other control elements, such as enhancers, and the like will be of particular use. A promoter may be derived in its entirety from a native gene or may be composed of different elements derived from different naturally occurring promoters. Alternatively, the promoter may include a synthetic polynucleotide sequence. Different promoters will direct the expression of a gene in different tissues or cell types, or at different stages of development, or in response to different environmental conditions or to the presence or the absence of a drug or transcriptional co-factor. Ubiquitous promoters are well known in the art.

In mammalian systems, three kinds of promoters exist and are candidates for construction of the expression vectors: (i) Pol I promoters that control transcription of large ribosomal RNAs; (ii) Pol II promoters that control the transcription of mRNAs (that are translated into protein) and small nuclear RNAs (snRNAs); (iii) and Pol III promoters that uniquely transcribe small non-coding RNAs. Each has advantages and constraints to consider when designing the construct for expression of the RNAs in vivo. For example, Pol III promoters are useful for synthesizing RNAi sequences (e.g., siRNA, shRNA, microRNA, or shmiRNA) from a DNA template in vivo. For greater control over tissue specific expression, Pol II promoters are preferred but can only be used for transcription of microRNAs.

Polynucleotides suitable for use with the compositions and methods described herein also include those that encode proteins under control of a mammalian regulatory sequence, such as, e.g., a promoter sequence and, optionally, an enhancer sequence. Exemplary promoters that are useful for the expression of proteins in mammalian cells include ubiquitous promoters such as an H1 promoter, a 7SK promoter, an apolipoprotein E-human α1-antitrypsin promoter (hAAT), a CK8 promoter, a murine U1 promoter (mU1a), an elongation factor 1α (EF-1α) promoter, an early growth response 1 (EGR1) promoter, a thyroxine binding globulin (TBG) promoter, a phosphoglycerate kinase (PGK) promoter, a CAG promoter, a chicken β-actin (CBA) promoter, an smCBA promoter, a CB7 promoter, a hybrid CMV enhancer/human β-actin promoter, a human β-actin promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), a CASI promoter, a dihydrofolate reductase (DHFR) promoter, a murine mammary tumor virus LTR promoter, an adenovirus major late (Ad MLP) promoter, a β-globin promoter (e.g., a minimal β-globin promoter), an HSV promoter (e.g., a minimal HSV ICPO promoter or a truncated HSV ICPO promoter), an SV40 promoter (e.g., an SV40 minimal promoter or an SV40 early promoter), a rous sarcoma virus (RSV) promoter, an eukaryotic translation initiation factor 4A1 (EIF4A1) promoter, a ferritin heavy (FerH) promoter, a ferritin light (FerL) promoter, a glyceraldehyde-3-phospohate dehydrogenase (GAPDH) promoter, a heat shock protein family A member 5 (HSPA5) gene, a heat shock protein family A member 4 (HSPA4) promoter, a ubiquitin B (UBB) promoter, and a U6 promoter.

Once a polynucleotide encoding the one or more proteins has been incorporated into the nuclear DNA of a mammalian cell, the transcription of this polynucleotide can be induced by methods known in the art. For example, expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode contain one or more therapeutic agents disclosed herein (e.g., a polynucleotide encoding an inner ear protein, an RNAi sequence, a miRNA, an antibody or an antigen-binding fragment thereof, or a component of a gene editing system, such as a nuclease) and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982). An enhancer may be spliced into a vector containing a heterologous polynucleotide, for example, at a position 5′ or 3′ to this gene. In a particular orientation, the enhancer is positioned at the 5′ side of the promoter, which in turn is located 5′ relative to the polynucleotide.

Additional regulatory elements that may be included in polynucleotides for use in the compositions and methods described herein are intron sequences. Intron sequences are non-protein-coding RNA sequences found in pre-mRNA which are removed during RNA splicing to produce the mature mRNA product. Intronic sequences are important for the regulation of gene expression in that they may be further processed to produce other non-coding RNA molecules. Alternative splicing, nonsense-mediated decay, and mRNA export are biological processes that have been shown to be regulated by intronic sequences. Intronic sequences may also facilitate the expression of a transgene through intron-mediated enhancement.

Further regulatory elements that may be used in conjunction with the vectors of the disclosure include inverted terminal repeat (ITR) sequences. ITR sequences are found, e.g., in AAV genomes at the 5′ and 3′ ends, each typically containing about 145 base pairs. AAV ITR sequences are particularly important for AAV genome multiplication by facilitating complementary strand synthesis once an AAV vector is incorporated into a cell. Moreover, ITRs have been shown to be critical for integration of the AAV genome into the genome of the host cell and encapsidation of the AAV genome.

Additional regulatory elements suitable for incorporation into the vectors of the disclosure include polyadenylation sequences (i.e., polyA sequences). PolyA sequences are RNA tails containing a stretch of adenine bases. These sequences are appended to the 3′ end of an RNA molecule to produce a mature mRNA transcript. Several biological processes related to mRNA processing and transport are modulated by polyA sequences, including nuclear export, translation, and stability. In mammalian cells, shortening of the polyA tails results in increased likelihood of mRNA degradation.

### Modulation of Inner Ear Immune Cells

The methods described herein can be used to modulate an inflammatory or immune response induced by administration of a nucleic acid vector of the disclosure (e.g., an AAV vector containing a polynucleotide encoding or containing a therapeutic agent (e.g., a polynucleotide encoding an inner ear protein, an RNAi sequence, an miRNA, an antibody or an antigen-binding fragment thereof, or a component of a gene editing system, such as a nuclease) operably linked to a ubiquitous promoter) in the inner ear of a subject or cell by co-administering to a subject or cell an inhibitor of inflammatory or immune signaling in a dose (e.g., an effective amount) and for a time sufficient to reduce or inhibit the immune response in the inner ear or a region thereof (e.g., semicircular canals, vestibule, and/or cochlea). These methods can be used to prevent undesirable immune toxicity in cells of the inner ear transduced with the nucleic acid vectors of the disclosure and improve therapeutic outcomes, e.g., improve hearing or vestibular function in a subject identified as having or at risk of developing hearing loss (e.g., sensorineural hearing loss) and/or vestibular dysfunction (e.g., vertigo, bilateral vestibulopathy, or oscillopsia). One way to modulate an immune response in the inner ear of a subject is to modulate an immune cell activity in the inner ear of the subject. This modulation can occur in vivo (e.g., in a human subject or animal model) or in vitro (e.g., in acutely isolated or cultured cells, such as human cells from a patient, repository, or cell line, or rodent cells).

### Inhibitors of Inflammatory or Immune Signaling

Agents suitable for use as inhibitors of inflammatory or immune signaling include anti-inflammatory agents (e.g., small molecules, peptides, and antibodies or fragments thereof), inhibitors of cell-mediated immunity (e.g., small molecules, peptides, antibodies or fragments thereof), and cellular de-targeting agents, such as, e.g., RNAi agents, immune cell microRNA target sequences, or nucleases. Inhibitors of inflammatory or immune signaling acting on tumor necrosis factor receptor super family (TNFRSF; e.g., TNFα) signaling, interferon (e.g., IFNγ) signaling, genes involved in antigen presentation, genes involved in allograft rejection, genes involved in apoptotic pathways, and genes involved in ligand-receptor interactions between immune cells and immune cells or immune cells and non-immune cells (e.g., inner ear cells) may be particularly useful in conjunction with the disclosed methods for reducing or inhibiting inflammatory or immune toxicity in the inner ear. In some embodiments, the aforementioned genes include C-C motif chemokine ligand 8 (CCL8), bone marrow stromal cell antigen 2 (BST2), beta-2-microglobulin (B2M), histocompatibility 2, Q region locus 6 (H2-Q6), histocompatibility 2, T region locus 23 (H2-T23), proteasome 20S subunit beta 9 (PSMB9), integral membrane protein 2B (ITM2B), histocompatibility 2, class II, locus MB1 (H2-DMB1), small secreted protein interferon-induced (AW112010), histocompatibility 2, K region locus 1 (H2-K1), histocompatibility 2, D region locus 1 (H2-D1), CD74 molecule (CD74), histocompatibility 2, class II antigen A (H2-AA), BPI fold containing family A member 1 (BPIFA1), histocompatibility 2, class II antigen A, beta 1 (H2-AB1), FAS, RELA, TNF, CD86, CXCR3, and TNFSF13. These genes can be inhibited using RNAi agents (e.g., short interfering RNA (siRNA), short hairpin RNA (shRNA), microRNA-adapted shRNA (shmiRNA), or antisense oligonucleotides (ASO)), inhibitory antibodies, peptide-based inhibitors, or small molecule inhibitors.

Anti-Inflammatory Agents and Inhibitors of Cell-Mediated Immunity

Anti-inflammatory agents and cell-mediated immunity inhibitor drugs suitable for use with the disclosed compositions and methods include, e.g., a corticosteroid or a nonsteroidal anti-inflammatory medication (NSAID, e.g., aspirin, ibuprofen, ketoprofen, and naproxen). In some embodiments, the anti-inflammatory agent is prednisone, prednisolone, methylprednisolone, methotrexate, hydroxychloroquine, sulfasalazine, leflunomide, cyclophosphamide, azathioprine, or a biologic such as tofacitinib, adalimumab, abatacept, anakinra, certolizumab, etanercept, golimumab, infliximab, rituximab, or tocilizumab. In some embodiments, the anti-inflammatory agent or cell-mediated immunity inhibitor is 6-mercaptopurine, 6-thioguanine, abatacept, adalimumab, alemtuzumab (Lemtrada), an aminosalicylate (5-aminoalicylic acid, sulfasalazine, mesalamine, balsalazide, olsalazine), celecoxib, diclofenac, diflunisal, etodolac, indomethacin, ketoprofen, ketorolac, nabumetone, oxaprozin, piroxicam, salsalate, sulindac, tolmetin, an antibiotic, an anti-histamine, a TNF inhibitor, such as a TNFα, TNFRS1 A/B, or TNFRSF13 A/B inhibitor (e.g., infliximab, adalimumab, certolizumab pegol, natalizumab, etanercept, golimumab, thalidomide, lenalidomide, pomalidomide, pentoxifylline, bupropion, or a 5-HT2A receptor agonist such as (R)-DOI, TCB-2, LSD, and LA-SS-Az), a CD74 inhibitor (e.g., milatuzumab, DRα1-MOG-35-35, RTL1000, C36L1), a CCL8 inhibitor, an interferon inhibitor (e.g., BX795, MRT68844, MRT67307, TPCA-1, Cyt387, AZD1480, ruxolitinib, tofacitinib, zinc, rocaglamide, mesopram, GIT27, StA-IFN-1, StA-IFN-2, StA-IFN-4, and StA-IFN-5 as described in Gage et al. J Biomol Screen. 21:978-88, 2016), anti-CD3 (muromonab), anti-IL2, Atgam®, thymoglobulin, azathioprine, belimumab, beta interferon, a calcineurin inhibitor, certolizumab, cromolyn, cyclosporin A, cyclosporine, myriocin, dimethyl fumarate (Tecfidera®), etanercept, fingolimod (Gilenya®), a fumaric acid ester, glatiramer acetate (Copaxone), golimumab, hydroxyurea, IFNγ, IFNβ, IL-11, infliximab, basiliximab, daclizumab, leflunomide, leukotriene receptor antagonist, long-acting beta2 agonist, methotrexate, mitoxantrone, mycophenolate mofetil, natalizumab (Tysabri®), ocrelizumab, a retinoid, rituximab, salicylic acid, short-acting beta2 agonist, tacrolimus, sirolimus, everolimus, zotarolimus, teriflunomide (Aubagio®), theophylline, vedolizumab, baricitinib, upadacitinib, alefacept, tocilizumab, ustekinumab (anti-IL12/IL23), or vedolizumab (Anti-alpha3 beta7 integrin).

Cellular De-Targeting Agents

The present disclosure additionally provides cellular de-targeting agents capable of preventing targeting, binding, internalization, and/or expression of the nucleic acid vectors of the disclosure (e.g., AAV vectors) in immune cells (e.g., inner ear immune cells). Without wishing to be bound by theory, cellular de-targeting agents disclosed herein can block the ability of the expression vector to deliver and/or express its payload in immune cells, thereby precluding potentially toxic immune cell activity within the inner ear and improving the therapeutic efficacy of the expression vector by restricting its expression to non-immune inner ear cells. Alternatively, cellular de-targeting agents may reduce or inhibit the activity and/or expression of pro-inflammatory molecules or immune mediators in immune cells of the inner ear. Non-limiting examples of cellular de-targeting agents include, e.g., nucleic acid sequences containing microRNA target sequences for microRNAs expressed by immune cells (e.g., activated immune cells, such as, e.g., activated macrophages or T cells) and RNAi agents, such as, e.g., short interfering RNA (siRNA), short hairpin RNA (shRNA), microRNA-adapted shRNA (shmiRNA), and antisense oligonucleotides (ASO), that are capable of targeting mRNAs encoding inflammatory or immune-mediator proteins in the inner ear.

In some embodiments, the cellular de-targeting agent is a nucleic acid sequence containing one or more microRNA target sequences for microRNAs expressed by immune cells (e.g., activated immune cells, such as, e.g., activated macrophages or T cells). A polynucleotide that can be transcribed to produce a nucleic acid sequence containing one or more microRNA target sequences for microRNAs expressed by immune cells can be incorporated into a nucleic acid vector (e.g., an AAV vector) containing a ubiquitous promoter operably linked to a therapeutic agent (e.g., a polynucleotide encoding an inner ear protein, an RNAi sequence, an miRNA, an antibody or an antigen-binding fragment thereof, or a component of a gene editing system, such as a nuclease). In some embodiments, the polynucleotide that can be transcribed to produce the one or more microRNA target sequences is operably linked to the same ubiquitous promoter that is used to express the therapeutic agent. In some embodiments, the polynucleotide that can be transcribed to produce the one or more microRNA target sequences is operably linked to a different promoter, such as an immune cell-specific promoter. In some embodiments, the immune cell is a macrophage or a T-cell. In some embodiments, the immune cell microRNA is miR-9, miR-15a/16, miR-21, miR-23a, miR-24, miR-29a, let-7, miR-98, miR-106a, miR-125a-99b˜let-7e cluster, miR-125b, miR-126, miR-127, miR-142, miR-145, miR-146a/b, miR-147b, miR-150, miR-155, miR-181, miR-187, miR-212, miR-222, miR-223, miR-451, miR-511, miR-720, miR-886-5p, and miR-4661, hsa-miR-378_st, hsa-miR-31_st, hsa-miR-935_st, hsa-miR-143_st, hsa-miR-362-5p_st. hsa-miR-532-5p_st, hsa-miR-500-star_st, hsa-miR-663_st, hsa-miR-125a-5p_st, hsa-miR-150_st, HBII-239_st, HBII-429_st, HBII-202_st, U27_st, U95_st, hsa-miR-768-5p_st, hsa-miR-223_st, or hsa-miR-652_st.

In some embodiments, the cellular de-targeting agent is an RNAi agent (e.g., siRNA, shRNA, shmiRNA, or ASO) capable of targeting an mRNA encoding a protein that is transduction-permissive with respect to a nucleic acid vector disclosed herein (e.g., AAV vector), particularly with respect to transduction of immune cells described herein. For example, several proteins have been identified in mammalian cells that facilitate or permit the transduction of AAV vectors into the cells. Non-limiting examples of transduction-permissive proteins include dynamin 1-3 (DNM1, DNM2, and DNM3), ADP ribosylation factor 1 (ARF1), cell division cycle 42 (CDC42), rho GTPase activating protein 26 (GRAF1), rac family small GTPase (RAC1), syntaxin 5 (STX5), phospholipase A2 (PLA2), importin-31 (KPNB1), MRE11 homolog double strand break repair nuclease (MRE11), RAD50 double strand break repair protein (RAD50), nibrin (NBN), FKBP prolyl isomerase 4 (FKBP4), chromodomain Y-linked 2A (CDY2), BAGE family member 2 (BAGE2), component of inhibitor of nuclear factor kappa B kinase complex (CHUK), late cornified envelope 1E (LCE1E), tuftelin 1 (TUFT1), poly(ADP-ribose) polymerase family member 8 (PARP8), testis expressed 47 (TEX47), catenin alpha 3 (CTNNA3), RUS family member 1 (RUSF1), cation channel sperm associated 3 (CATSPER3), CDC like kinase 2 (CLK2), FXYD domain containing ion transport regulator 2 (FXYD2), ADAM metallopeptidase with thrombospondin type 1 motif 17 (ADAMTS17), forkhead box A1 (FOXA1), translocase of inner mitochondrial membrane 44 (TIMM44), protein kinase AMP-activated non-catalytic subunit gamma 3 (PRKAG3), transforming growth factor beta 1 (TGFB1), chromosome 1 open reading frame 158 (C1ORF158), collagen type XX alpha 1 chain (COL20A1), ras-related protein rab-9a (RAB9A), chromosome 1 open reading frame 210 (C1ORF210), phosphatidylinositol 4-kinase alpha (PI4KA), acyl-CoA synthetase medium chain family member (ACSM5), calcium voltage-gated channel subunit 1 alpha (CACNA1A), zinc finger CCHC-type containing 7 (ZCCHC7), solute carrier family 31 member 2 (SLC31A2), ras-related protein rab-34 (RAB34), solute carrier family 7 member 14 (SLC7A14), BRCA1 associated ATM activator 1 (BRAT1), pterin-4 alpha-carbinolamine dehydratase 1 (PCBD), keratin 83 (KRT83), autophagy related 9A (ATG9A), mediator complex subunit 4 (MED4), solute carrier family 25 member 47 (SLC25A47), rho GTPase activating protein 1 (ARHGAP9), FXYD domain containing ion transport regulator 1 (FXYD1), PH domain and leucine rich repeat protein phosphatase 1 (PHLPP1), PTPN13 like Y-linked (PRY), C-C motif chemokine receptor 6 (CCR6), and any one of the other transduction-permissive proteins described in Mano et al. (PNAS, 112:1276-81, 2015), the disclosure of which is incorporated by reference herein in its entirety.

In some embodiments, expression of a transduction-permissive protein is inhibited selectively in immune cells by, e.g., delivering a nucleic acid vector encoding at least an siRNA, shRNA, shmiRNA, ASO, or a guide sequence-nuclease complex (e.g., guide-CRISPR-Cas9) targeting an mRNA encoding any one of the aforementioned transduction-permissive proteins under regulatory control of an immune cell-specific promoter.

In some embodiments, the immune cell-specific promoter is a macrophage-specific promoter. In some embodiments, the macrophage-specific promoter is a colony stimulating factor 1 (CSF-1) promoter, colony stimulating factor 1 receptor (CSF1R) promoter, CD68 molecule (CD68) promoter, CD4 molecule (CD4) promoter, CD2 molecule (CD2) promoter, C-X3-C motif chemokine receptor 1 (CX3CR1) promoter, microfibril associated protein 4 (MFAP4) promoter, macrophage scavenger receptor 1 (MSR1) promoter, integrin subunit alpha M (ITGAM) promoter, integrin subunit alpha X (ITGAX) promoter, CD207 molecule (CD207) promoter, adhesion G protein-coupled receptor E1 (ADGRE1) promoter, or SP146-C1 promoter (see Kang et al. Gene Ther. 21:353-62, 2014).

In some embodiments, the immune cell-specific promoter is a T cell-specific promoter. In some embodiments, the T cell-specific promoter is a CD4 molecule (CD4) promoter, CD8 molecule (CD8) promoter, CD69 molecule (CD69) promoter, tumor necrosis factor alpha (TNFα) promoter, interleukin 2 (IL-2) promoter, C-X-C- motif chemokine receptor 3 (CXCR3) promoter, T-Box transcription factor 21 (TBX21) promoter, interleukin 4 (IL-4) promoter, interleukin 5 (IL-5) promoter, interleukin 9 (IL-9) promoter, interleukin 10 (IL-10) promoter, interleukin 17 (IL-17) promoter, C-C motif chemokine receptor 4 (CCR4) promoter, C-C motif chemokine receptor 6 (CCR6), GATA binding protein 3 (GATA3) promoter, interferon regulatory factor 4 (IRF4) promoter, killer cell lectin like receptor B1 (KLRB1), RAR related orphan receptor C (RORC) promoter, Fc fragment of IgG receptor IIIa (FCGR3A), T cell beta-chain (TCRβ) promoter, or CIFT promoter (Fang et al. Mol Ther Methods Clin Develop. 19:14-23, 2020), human CD3delta promoter (Ji et al. J Biol Chem. 277:47898-906, 2002).

Co-Administration of Nucleic Acid Vectors with Inhibitors of Inflammatory or Immune Signaling

According to the methods disclosed herein, a nucleic acid vector containing a ubiquitous promoter operably linked to a polynucleotide encoding or containing a therapeutic agent (e.g., a polynucleotide encoding an inner ear protein, an RNAi sequence, an miRNA, an antibody or an antigen-binding fragment thereof, or a component of a gene editing system, such as a nuclease) can be administered in combination with one or more inhibitors of inflammatory or immune signaling to an inner ear of a subject (e.g., a human) to treat a disorder of the inner ear, such as hearing loss (e.g., sensorineural hearing loss), tinnitus, or vestibular dysfunction.

In some embodiments, the inhibitor of inflammatory or immune signaling is a polynucleotide (e.g., siRNA, shRNA, shmiRNA, ASO, guide-nuclease construct, antibody, or peptide) described above. In some embodiments, the polynucleotide inhibitor of inflammatory or immune signaling is a naked polynucleotide (i.e., a polynucleotide not contained in an expression vector). In some embodiments, the polynucleotide inhibitor of inflammatory or immune signaling is incorporated into a nucleic acid expression vector (e.g., vector). In some embodiments, the polynucleotide inhibitor of inflammatory or immune signaling is incorporated into an expression vector containing a transgene encoding or containing a therapeutic agent (e.g., an inner ear protein, peptide, antibody or antigen-binding fragment thereof, RNAi sequence, miRNA, or a nuclease) operably linked to a ubiquitous promoter. In some embodiments, the expression vector includes a polycistronic expression cassette containing a transgene containing or encoding a therapeutic agent (e.g., an inner ear protein, peptide, antibody or antigen-binding fragment thereof, RNAi sequence, miRNA, or a nuclease) operably linked to a ubiquitous promoter and a transgene containing or encoding the polynucleotide inhibitor of inflammatory or immune signaling. In some embodiments, the transgene encoding or containing a therapeutic agent (e.g., an inner ear protein, peptide, antibody or antigen-binding fragment thereof, RNAi sequence, miRNA, or a nuclease) and the transgene containing or encoding the polynucleotide inhibitor of inflammatory or immune signaling are under regulatory control of a single promoter sequence (e.g., a ubiquitous promoter disclosed herein). In some embodiments, the transgene encoding a therapeutic agent (e.g., an inner ear protein, peptide, antibody or antigen-binding fragment thereof, RNAi sequence, miRNA, or a nuclease) and the transgene containing or encoding the polynucleotide inhibitor of inflammatory or immune signaling are each under regulatory control of a dedicated promoter sequence, wherein the transgene encoding the therapeutic agent is under control of a ubiquitous promoter and the transgene encoding the inhibitor of inflammatory or immune signaling is under control of an immune cell-specific promoter. In some embodiments, the transgene encoding the therapeutic agent (e.g., an inner ear protein, peptide, antibody or antigen-binding fragment thereof, RNAi sequence, miRNA, or a nuclease) and the transgene containing or encoding the polynucleotide inhibitor of inflammatory or immune signaling are each contained in separate nucleic acid vectors (e.g., AAV vectors), wherein the transgene encoding the therapeutic agent is under control of a ubiquitous promoter and the transgene encoding the inhibitor of inflammatory or immune signaling is under control of an immune cell-specific promoter.

In some embodiments, the inhibitor of inflammatory or immune signaling is a small molecule, peptide, RNAi sequence, miRNA target sequence, nuclease, or antibody or antigen-binding fragment thereof.

In some embodiments, the expression vector that includes a polycistronic expression cassette containing a transgene encoding or containing a therapeutic agent (e.g., an inner ear protein, peptide, antibody or antigen-binding fragment thereof, RNAi sequence, miRNA, or a nuclease) and a transgene containing or encoding the polynucleotide inhibitor of inflammatory or immune signaling is administered to a subject (e.g., a human) having or at risk of developing an inner ear disorder, such as, e.g., hearing loss (e.g., sensorineural hearing loss), tinnitus, or vestibular dysfunction. In some embodiments, the subject with an inner ear disorder is administered a combination of an expression vector containing the transgene encoding or containing a therapeutic agent (e.g., an inner ear protein, peptide, antibody or antigen-binding fragment thereof, RNAi sequence, miRNA, or a nuclease) and an expression vector containing a transgene containing or encoding the polynucleotide inhibitor of inflammatory or immune signaling. In some embodiments, the subject with an inner ear disorder is administered a combination of an expression vector containing the transgene encoding or containing a therapeutic agent (e.g., an inner ear protein, peptide, antibody or antigen-binding fragment thereof RNAi sequence, miRNA, or a nuclease) and an inhibitor of inflammatory or immune signaling that is a small molecule, peptide, RNAi sequence, or antibody or antigen-binding fragment thereof.

In some embodiments, the expression vector containing the transgene encoding or containing a therapeutic agent (e.g., an inner ear protein, peptide, antibody or antigen-binding fragment thereof, RNAi sequence, miRNA, or a nuclease) under control of a ubiquitous promoter and the inhibitor of inflammatory or immune signaling are administered at the same time (e.g., administration of all agents occurs within 15 minutes, 10 minutes, 5 minutes, 2 minutes or less). The expression vector containing the transgene encoding or containing a therapeutic agent (e.g., an inner ear protein, peptide, antibody or antigen-binding fragment thereof, RNAi sequence, miRNA, or a nuclease) and the inhibitor of inflammatory or immune signaling can be administered simultaneously via co-formulation. The transgene encoding the therapeutic agent and the inhibitor of inflammatory or immune signaling can also be administered sequentially, such that the action of the two agents overlaps and their combined effect is such that the reduction in a symptom, or other parameter related to the disorder is greater than what would be observed with either treatment delivered alone or in the absence of the other. The effect of the two or more treatments can be partially additive, wholly additive, or greater than additive (e.g., synergistic). Sequential or substantially simultaneous administration of each therapeutic transgene and the inhibitor of inflammatory or immune signaling can be performed by any appropriate route including, but not limited to intravenous routes, intramuscular routes, and local routes (e.g., delivery to the inner ear). The therapeutic agents can be administered by the same route or by different routes. For example, the expression vector containing a transgene encoding or containing a therapeutic agent (e.g., an inner ear protein, peptide, antibody or antigen-binding fragment thereof, RNAi sequence, miRNA, or a nuclease) may be administered locally into the inner ear, while the inhibitor of inflammatory or immune signaling can be administered systemically (e.g., via intravenous injection or oral administration). In some embodiments, the expression vector containing a transgene encoding or containing a therapeutic agent (e.g., an inner ear protein, peptide, antibody or antigen-binding fragment thereof, RNAi sequence, miRNA, or a nuclease) and the inhibitor of inflammatory or immune signaling can both be administered locally into the inner ear. In some embodiments, the expression vector containing a transgene encoding or containing a therapeutic agent (e.g., an inner ear protein, peptide, antibody or antigen-binding fragment thereof, RNAi sequence, miRNA, or a nuclease) and the inhibitor of inflammatory or immune signaling are simultaneously administered locally into the inner ear. In some embodiments, the expression vector containing a transgene encoding or containing a therapeutic agent (e.g., an inner ear protein, peptide, antibody or antigen-binding fragment thereof, RNAi sequence, miRNA, or a nuclease) is administered to locally to the inner ear prior to the administration of the inhibitor of inflammatory or immune signaling locally into the inner ear. In some embodiments, the expression vector containing a transgene encoding or containing a therapeutic agent (e.g., an inner ear protein, peptide, antibody or antigen-binding fragment thereof, RNAi sequence, miRNA, or a nuclease) is administered locally to the inner ear following the administration of the inhibitor of inflammatory or immune signaling locally into the inner ear. In some embodiments, the expression vector containing a transgene encoding or containing a therapeutic agent (e.g., an inner ear protein, peptide, antibody or antigen-binding fragment thereof, RNAi sequence, miRNA, or a nuclease) is administered locally to the inner ear at the same time as the systemic (e.g., intravenous or oral) administration of the inhibitor of inflammatory or immune signaling. In some embodiments, the expression vector containing a transgene encoding or containing a therapeutic agent (e.g., an inner ear protein, peptide, antibody or antigen-binding fragment thereof, RNAi sequence, miRNA, or a nuclease) is administered locally to the inner ear prior to the systemic (e.g., intravenous or oral) administration of the inhibitor of inflammatory or immune signaling. In some embodiments, the expression vector containing a transgene encoding or containing a therapeutic agent (e.g., an inner ear protein, peptide, antibody or antigen-binding fragment thereof, RNAi sequence, miRNA, or a nuclease) is administered locally to the inner ear following the systemic administration of the inhibitor of inflammatory or immune signaling. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent and vice-versa.

### Immune Cell Types

The types of immune cells that can be modulated include T cells (e.g., peripheral T cells, cytotoxic T cells/CD8+ T cells, T helper cells/CD4+ T cells, memory T cells, regulatory T cells/Tregs, natural killer T cells/NKTs, mucosal associated invariant T cells, and gamma delta T cells), B cells (e.g., memory B cells, plasmablasts, plasma cells, follicular B cells/B-2 cells, marginal zone B cells, B-1 cells, regulatory B cells/Bregs), dendritic cells (e.g., myeloid DCs/conventional DCs, plasmacytoid DCs, or follicular DCs), granulocytes (e.g., eosinophils, mast cells, neutrophils, and basophils), monocytes, macrophages (e.g., peripheral macrophages or tissue resident macrophages), myeloid-derived suppressor cells, natural killer (NK) cells, innate lymphoid cells (e.g., ILC1s, ILC2s, and ILC3s), thymocytes, and megakaryocytes.

### Modulation of Immune Cell Activities

The immune cell activities that can be modulated by administering to a subject or contacting a cell with an effective amount of an inhibitor of inflammatory or immune signaling described herein include activation (e.g., macrophage, T cell, NK cell, ILC, B cell, dendritic cell, neutrophil, eosinophil, or basophil activation), phagocytosis (e.g., macrophage, neutrophil, monocyte, mast cell, B cell, eosinophil, or dendritic cell phagocytosis), antibody-dependent cellular phagocytosis (e.g., ADCP by monocytes, macrophages, neutrophils, or dendritic cells), antibody-dependent cellular cytotoxicity (e.g., ADCC by NK cells, ILCs, monocytes, macrophages, neutrophils, eosinophils, dendritic cells, or T cells), polarization (e.g., macrophage polarization toward an M1 phenotype or T cell polarization), proliferation (e.g., proliferation of B cells, T cells, monocytes, macrophages, dendritic cells, NK cells, ILCs, mast cells, neutrophils, eosinophils, or basophils), recruitment (e.g., recruitment of B cells, T cells, monocytes, macrophages, dendritic cells, NK cells, ILCs, mast cells, neutrophils, eosinophils, or basophils), migration (e.g., migration of B cells, T cells, monocytes, macrophages, dendritic cells, NK cells, ILCs, mast cells, neutrophils, eosinophils, or basophils), differentiation (e.g., regulatory T cell differentiation), immune cell cytokine production (e.g., pro-inflammatory cytokine production), antigen presentation (e.g., dendritic cell, macrophage, and B cell antigen presentation), maturation (e.g., dendritic cell maturation), and degranulation (e.g., mast cell, NK cell, ILCs, cytotoxic T cell, neutrophil, eosinophil, or basophil degranulation). Modulation can decrease these activities to reduce inflammation and/or toxicity in the inner ear.

In some embodiments, an effective amount of the inhibitor of inflammatory or immune signaling is an amount sufficient to decrease one or more (e.g., 2 or more, 3 or more, 4 or more) of the following immune cell activities in the subject or cell: T cell polarization; T cell activation; dendritic cell activation; neutrophil activation; eosinophil activation; basophil activation; T cell proliferation; B cell proliferation; T cell proliferation; monocyte proliferation; macrophage proliferation; dendritic cell proliferation; NK cell proliferation; ILC proliferation, mast cell proliferation; neutrophil proliferation; eosinophil proliferation; basophil proliferation; cytotoxic T cell activation; circulating monocytes; peripheral blood hematopoietic stem cells; macrophage polarization (e.g., toward an M1 phenotype); macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; ILC activation, ILC ADCC, ILC degranulation, cytotoxic T cell degranulation; neutrophil degranulation; eosinophil degranulation; basophil degranulation; neutrophil recruitment; eosinophil recruitment; NKT cell activation; B cell activation; regulatory T cell differentiation; or dendritic cell maturation. In some embodiments the immune response in the inner ear (e.g., an immune cell activity in the inner ear listed herein) is decreased in the inner ear of subject or an inner ear cell at least by 1%, 2%, 5%, 10%, 15%, 20%, 25%, 30%, 35%, 40%, 50%, 60%, 70%, 80%, 100%, 150%, 200%, 300%, 400%, 500% or more, compared to before the administration of the disclosed composition. In some embodiments the immune response is decreased in the inner ear of subject or an inner ear cell between 5-20%, between 5-50%, between 10-50%, between 20-80%, between 20-70%, between 50-200%, between 100%-500%.

### Methods of Measuring Immune Cell Activities

After an inhibitor of inflammatory or immune signaling is administered to treat a subject or contact a cell, a readout can be used to assess the effect on inner ear immune cell activity. Inner ear immune cell activity can be assessed by measuring a cytokine or marker associated with a particular immune cell type (e.g., a macrophage). In some embodiments, the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In some embodiments, the parameter is increased or decreased in the subject between 5-20%, between 5-50%, between 10-50%, between 20-80%, between 20-70%, between 50-200%, between 100%-500%. An inhibitor of inflammatory or immune signaling can be administered at a dose (e.g., an effective amount) and for a time sufficient to modulate an inner ear immune cell activity described herein below.

After an inhibitor of inflammatory or immune signaling is administered to treat a subject or contact a cell, a readout can be used to assess the effect on immune cell migration. Inner ear immune cell migration can be assessed by measuring the number of immune cells in a location of interest (e.g., site of inflammation in the inner ear). Inner ear cell immune cell migration can also be assessed by measuring a chemokine, receptor, or marker associated with inner ear immune cell migration. In some embodiments the parameter is increased or decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In some embodiments, the parameter is increased or decreased in the subject between 5-20%, between 5-50%, between 10-50%, between 20-80%, between 20-70%, between 50-200%, between 100%-500%. An inhibitor of inflammatory or immune signaling can be administered at a dose (e.g., an effective amount) and for a time sufficient to reduce inner ear immune cell migration (e.g., migration of immune cells into the inner ear or to a site of inflammation in the inner ear).

In some embodiments, an inhibitor of inflammatory or immune signaling described herein reduces apoptosis of non-immune inner ear cells (e.g., hair cells or supporting cells), reduces off-target expression of therapeutic transgenes in inner ear immune cells, inhibits activation of antigen-directed immunity mediated by major histocompatibility complex (MHC), T cell genes, and macrophage genes, reduces the number of immune cells (e.g., monocytes and dendritic cells) in the inner ear, and/or reduces ligand-receptor interactions, such as, e.g., TNFRSF-mediated interactions involving neutrophils and granulocytes. In some embodiments, an inhibitor of inflammatory or immune signaling described herein decreases one or more of macrophage migration, macrophage proliferation, macrophage recruitment, macrophage lymph node egress, macrophage differentiation, macrophage activation, macrophage polarization (e.g., polarization toward an M1 phenotype), macrophage cytokine production (e.g., pro-inflammatory cytokine production), macrophage maturation, macrophage antigen presentation, macrophage ADCC, or macrophage ADCP. In some embodiments, the cytokine is a pro-inflammatory cytokine (e.g., tumor necrosis factor α (TNFα) or interferon gamma (IFNγ)). In some embodiments, an inhibitor of inflammatory or immune signaling described herein decreases inflammation, auto-antibody levels, or the rate or number of flare-ups of inflammation or cell-mediated immunity in the inner ear of a subject. In some embodiments, the macrophage is an M1 macrophage.

### Immune Effects

A variety of known in vitro and in vivo assays can be used to determine how an inhibitor of inflammatory or immune signaling affects inner ear immune cell activity. For example, the effect of an inhibitor of inflammatory or immune signaling on immune cell activation can be assessed through measurement of secreted cytokines and chemokines. An activated inner ear immune cell (e.g., T cell, B cell, macrophage, monocyte, dendritic cell, eosinophil, basophil, mast cell, NK cell, ILC, or neutrophil) can produce pro-inflammatory cytokines and chemokines (e.g., IL-1β, IL-5, IL-6, IL-8, IL-10, IL-12, IL-13, IL-18, TNFα, and IFN-γ). Activation can be assessed by measuring cytokine levels in an inner ear tissue or fluid sample from a human subject or animal model, with lower pro-inflammatory cytokine or chemokine levels indicating decreased immune cell activation following treatment with an inhibitor of inflammatory or immune signaling. Activation can also be assessed in vitro by measuring cytokines secreted into the media by cultured inner ear immune cells. Cytokines can be measured using ELISA, western blot analysis, and other approaches for quantifying secreted proteins. Other measures of inflammatory or immune cell signaling, such as immune cell proliferation, migration, maturation, degranulation, activation, etc. can similarly be measured using methods known in the art.

The effect of an inhibitor of inflammatory or immune signaling on inner ear immune cell cytokine production can be assessed by evaluation of cellular markers in an immune cell sample obtained from an inner ear of a human subject or animal model. An inner ear tissue or fluid sample can be collected from the subject and evaluated for one or more (e.g., 1, 2, 3, 4, or 4 or more) cytokine markers selected from: pro-inflammatory cytokines (e.g., IL-1β, IL-5, IL-6, IL-8, IL-10, IL-12, IL-13, IL-17, IL-18, IL-22, TNFα, IFNγ, GMCSF), pro-survival cytokines (e.g., IL-2, IL-4, IL-6, IL-7, and IL-15) and anti-inflammatory cytokines (e.g., IL-4, IL-10, IL-11, IL-13, IFNα, and TGFβ). Some cytokines can function as both pro- and anti-inflammatory cytokines depending on context or indication (e.g., IL-4 is often categorized as an anti-inflammatory cytokine but plays a pro-inflammatory role in mounting an allergic or anti-parasitic immune response). Cytokines can be also detected in the culture media of inner ear immune cells contacted with an inhibitor of inflammatory or immune signaling. Cytokines can be detected using ELISA, western blot analysis, or other methods for detecting protein levels in solution. The effect of an inhibitor of inflammatory or immune signaling can be determined by comparing results from before and after inhibitor of inflammatory or immune signaling administration.

### Expression of Exogenous Polynucleotides in Mammalian Cells

Mutations in a variety of genes, such as Myosin 7A (MYO7A), POU Class 4 Homeobox 3 (POU4F3), Solute Carrier Family 17 Member 8 (SLC17A8), Gap Junction Protein Beta 2 (GJB2), Claudin 14 (CLDN14), Cochlin (COCH), Protocadherin Related 15 (PCDH15), and Transmembrane 1 (TMC1), have been linked to sensorineural hearing loss and/or deafness, and some of these mutations, such as mutations in MYO7A, POU4F3, and COCH are also associated with vestibular dysfunction. The compositions and methods described herein can be used to induce or increase the expression of a protein encoded by a polynucleotide (e.g., a nucleic acid corresponding to a gene expressed in healthy inner ear cells, such as the wild-type form of a gene implicated in hearing loss and/or vestibular dysfunction, or a gene involved in inner ear cell development, function, cell fate specification, regeneration, survival, proliferation, and/or maintenance) in inner ear cells (e.g., human inner ear cells). Nucleic acid vectors (e.g., AAV vectors containing a wild-type AAV capsid or rAAV vectors) containing the polynucleotide under regulatory control of a ubiquitous promoter can be administered to a human subject (e.g., to the inner ear of the subject) to induce or increase expression of the protein encoded by the polynucleotide in one or more inner ear cell types. A wide array of methods has been established for the delivery of proteins to human cells and for the stable expression of polynucleotides encoding proteins in human cells.

The nucleic acid vectors described herein can be used to express a polynucleotide in one or more inner ear cell types while mitigating undesirable immune toxicity. In some embodiments, the vectors described herein can be used to express two or more (e.g., 2, 3, 4, or more) polynucleotides in one or more cell types. A list of inner ear cell types and polynucleotides that can be expressed in each cell type are provided in Table 1, below. Accession numbers for the polynucleotides of Table 1 are provided in Table 2.

TABLE 1
Polynucleotides that can be
expressed in one or more inner ear cell types

| Cell type | Polynucleotide |
|---|---|
| Inner hair | Otoferlin (Otof), Soluble Carrier Family 17 Member 8 |
| cells (IHCs) | (Slc17a8, also known as Vglut3) |
| Outer hair | Stereocilin (Strc), Cholinergic Receptor Nicotinic Alpha |
| cells (OHCs) | 9 Subunit (Chrna9), Cholinergic Receptor Nicotinic |
| | Alpha 10 Subunit (Chrna10), Oncomodulin (Ocm) |
| IHCs and | Whirlin (Whrn) |
| vestibular | |
| hair cells | |
| Cochlear | Atonal BHLH Transcription Factor 1 (Atoh1), POU |
| hair cells | Class 4 Homeobox 3 (Pou4f3), Growth Factor |
| (IHCs and | Independent 1 Transcriptional Repressor (Gfi1), ISL |
| OHCs) | LIM Homeobox 1 (Isl1), Clarin 1 (Clrn1), |
| | Protocadherin Related 15 (Pcdh15), Cadherin Related |
| | 23 (Cdh23), Myosin 7a (Myo7a), Transmembrane |
| | Channel Like 1 (Tmc1), Harmonin (Ush1c) |
| Cells of | Potassium Voltage-Gated Channel Subfamily Q |
| the stria | Member (Kcnq1), Potassium Voltage-Gated Channel |
| vascularis | Subfamily E Regulatory Subunit 1 (Kcne1), Gap |
| (SV) | Junction Protein Beta 2 (Gjb2), Gap Junction Protein |
| | Beta 6 (Gjb6), Tyrosinase (Tyr), a nuclease (e.g., |
| | CRISPR Associated Protein 9 (Cas9), Transcription |
| | Activator-Like Effector Nuclease (TALEN), Zinc |
| | Finger Nuclease (ZFN), or gRNA), a microRNA |
| | (miRNA, e.g., miR-183, miR-96, or miR-182) |
| Fibrocytes/ | Collagens (e.g., Collagen Type I Alpha 1 Chain |
| mesenchyme | (Col1a1), Collagen Type I Alpha 2 Chain (Col1a2), |
| | Collagen Type II Alpha 1 Chain (Col2a1), or other |
| | collagen genes) |
| Interdental | Carcinoembryonic Antigen Related Cell Adhesion |
| cells | Molecule 16 (Ceacam16), Otoancorin (Otoa), Gjb2, |
| | Gjb6 |
| Spiral | Solute Carrier Family 26 Member 4 (Slc26a4) |
| prominence | |
| cells | |
| Root cells | Slc26a4 |
| Cochlear and | SRY-Box 9 (Sox9), Spalt Like Transcription Factor 2 |
| vestibular | (Sall2), Calmodulin Binding Transcription Activator 1 |
| supporting | (Camta1), Hes Related Family BHLH Transcription |
| cells | Factor With YRPW Motif 2 (Hey2), Gata Binding |
| | Protein 2 (Gata2), Hes Related Family BHLH |
| | Transcription Factor With YRPW Motif 1 (Hey1), |
| | Ceramide Synthase 2 (Lass2), SRY-Box 10 (Sox10), |
| | GATA Binding Protein 3 (Gata3), Cut Like Homeobox |
| | 1 (Cux1), Nuclear Receptor Subfamily 2 Group F |
| | Member (Nr2f1), Hes Family BHLH Transcription |
| | Factor 1 (Hes1), RAR Related Orphan Receptor B |
| | (Rorb), Jun Proto-Oncogene AP-1 Transcription |
| | Factor Subunit (Jun), Zinc Finger Protein 667 (Zfp667), |
| | LIM Homeobox 3 (Lhx3), Nescient Helix-Loop-Helix |
| | 1 (Nhlh1), MAX Dimerization Protein 4 (Mxd4), Zinc |
| | Finger MIZ-Type Containing 1 (Zmiz1), Myelin |
| | Transcription Factor 1 (Myt1), Signal Transducer |
| | And Activator Of Transcription 3 |
| | (Stat3), BarH Like Homeobox 1 (Barhl1), Thymocyte |
| | Selection Associated High Mobility Group Box |
| | (Tox), Prospero Homeobox 1 (Prox1), Nuclear |
| | Factor I A (Nfia), Thyroid Hormone Receptor Beta |
| | (Thrb), MYCL Proto-Oncogene BHLH Transcription |
| | Factor (Mycl1), Lysine Demethylase 5A (Kdm5a), |
| | CAMP Responsive Element Binding Protein 3 Like 4 |
| | (Creb314), ETS Variant 1 (Etv1), Paternally Expressed |
| | 3 (Peg3), BTB Domain And CNC Homolog 2 (Bach2), |
| | ISL LIM Homeobox (Isl1), Zinc Finger And BTB |
| | Domain Containing 38 (Zbtb38), Limb Bud And Heart |
| | Development (Lbh), Tubby Bipartite Transcription |
| | Factor (Tub), Ubiquitin C (Hmg20), RE1 Silencing |
| | Transcription Factor (Rest), Zinc Finger Protein 827 |
| | (Zfp827), AF4/FMR2 Family Member 3 (Aff3), PBX/ |
| | Knotted 1 Homeobox 2 (Pknox2), AT-Rich Interaction |
| | Domain 3B (Arid3b), MLX Interacting Protein |
| | (Mixip), Zinc Finger Protein (Zfp532), IKAROS |
| | Family Zinc Finger 2 (Ikzf2), Spalt Like Transcription |
| | Factor 1 (Sall1), SIX Homeobox 2 (Six2), Spalt Like |
| | Transcription Factor 3 (Sall3), Lin-28 Homolog B |
| | (Lin28b), Pou4f3, Regulatory Factor X7 (Rfx7), |
| | Atoh1, a polynucleotide encoding an Atoh1 variant |
| | containing mutations at amino acids 328, 331, and/or |
| | 334 (e.g., S328A, S331A, S334A, S328A/S331A, |
| | S328A/S334A, S331A/S334A, and S328A/S331A/ |
| | S334, e.g., a polynucleotide encoding a variant having |
| | the sequence of any one of SEQ ID NOs: 1-7), Gfi1, |
| | SRY-Box 4 (Sox4), Brain Derived Neurotrophic |
| | Factor (Bdnf), Neurotrophin 3 (Ntf3), SRY-Box 11 |
| | (Sox11), TEA Domain Transcription Factor 2 (Tead2), |
| | Yes Associated Protein 1 (Yap1), a nuclease (e.g., |
| | Cas9, TALEN, ZFN, or gRNA), a microRNA |
| | (miRNA, e.g., miR-183, miR-96, or miR-182), |
| Vestibular | Bdnf, Ntf3, Transmembrane and Tetratricopeptide |
| and cochlear | Repeat Containing 4 (Tmtc4), a nuclease (e.g., Cas9, |
| hair cells | TALEN, ZFN, or gRNA), a microRNA (miRNA, e.g., |
| | miR-183, miR-96, or miR-182) |
| Border cells | Bdnf, Ntf3, Tectorin Beta (Tectb), Tectorin Alpha |
| (cochlear | (Tecta), Gjb2, Gjb6 |
| supporting | |
| cell subtype) | |
| Inner | Bdnf, Ntf3, Tectb, Tecta, Transmembrane Protein |
| phalangeal | 16A (Tmem16a), Gjb2, Gjb6 |
| cells | |
| (cochlear | |
| supporting | |
| cell subtype) | |
| Pillar cells | Nerve Growth Factor Receptor (Ngfr), Bdnf, Ntf3, |
| (cochlear | Tectb, Tecta, Gjb2, Gjb6 |
| supporting | |
| cell subtype) | |
| Deiters cells | Bdnf, Ntf3, Tectb, Tecta, Ikzf2, Gjb2, Gjb6 |
| (cochlear | |
| supporting | |
| cell subtype) | |
| Hensen's cells | Gjb2, Gjb6 |
| (cochlear | |
| supporting | |
| cell subtype) | |
| Claudius cells | Gjb2, Gjb6 |
| (cochlear | |
| supporting | |
| cell subtype) | |
| Spiral | Bdnf, Ntf3, a nuclease (e.g., Cas9, TALEN, ZFN, |
| ganglion | or gRNA), a microRNA (miRNA, e.g., miR-183, |
| neurons | miR-96, or miR-182), shRNA directed to RGMA, |
| (SGN) | |
| Scarpa's | Bdnf, Ntf3, shRNA directed to RGMA |
| ganglion | |
| All fibrocytes | Gjb2, Gjb6 |
| and epithelia | |
| Vestibular | Kcnq1, Kcne1, Slc26a4 |
| dark cells | |
| Glia | Peripheral Myelin Protein 22 (Pmp22), Bdnf, Ntf3, |
| | Myelin Protein Zero (Mpz) |

TABLE 2
Accession numbers for polynucleotides listed in Table 1

| | NCBI |
|---|---|
| | Accession |
| Gene name | number |
| Otof, Otoferlin (variant 1) | NM_194248 |
| Otof, Otoferlin (variant 2) | NM_004802 |
| Otof, Otoferlin (variant 3) | NM_194322 |
| Otof, Otoferlin (variant 4) | NM_194323 |
| Otof, Otoferlin (variant 5) | NM_001287489 |
| Vglut3, Vesicular glutamate transporter 3 (variant 1) | NM_139319 |
| Vglut3, Vesicular glutamate transporter 3 (variant 2) | NM_001145288 |
| Strc, Stereocilin | NM_153700 |
| Tmc1, Transmembrane channel like 1 | NM_138691 |
| Myo7a, Myosin VIIa (variant 1) | NM_000260 |
| Myo7a, Myosin VIIa (variant 2) | NM_001127180 |
| Harmonin (variant 1) | NM_005709 |
| Harmonin (variant b3) | NM_153676 |
| Harmonin (variant 3) | NM_001297764 |
| Whirlin (variant 1) | NM_015404 |
| Whirlin (variant 2) | NM_001083885 |
| Whirlin (variant 3) | NM_001173425 |
| Atoh1, Atonal BHLH transcription factor 1 | NM_005172 |
| Pou4f3, POU class 4 homeobox 3 | NM_002700 |
| Gfi1, Growth factor independent 1 transcriptional | NM_005263 |
| repressor (variant 1) | |
| Gfi1, Growth factor independent 1 transcriptional | NM_001127215 |
| repressor (variant 2) | |
| Gfi1, Growth factor independent 1 transcriptional | NM_001127216 |
| repressor (variant 3) | |
| Isl1, ISL LIM homeobox 1 | NM_00220 |
| Clrn1, Clarin 1 (variant 1) | NM_174878 |
| | NM_174879 |
| Clrn1, Clarin 1 (variant 4) | NM_052995 |
| Clrn1, Clarin 1 (variant 5) | NM_001195794 |
| Clrn1, Clarin 1 (variant 6) | NM_001256819 |
| Pcdh15, Protocadherin related 15 | NM_033056 |
| Cdh23, Cadherin related 23 (variant 1) | NM_022124 |
| Cdh23, Cadherin related 23 (variant 2) | NM_052836 |
| Cdh23, Cadherin related 23 (variant 3) | NM_001171930 |
| Cdh23, Cadherin related 23 (variant 4) | NM_001171931 |
| Cdh23, Cadherin related 23 (variant 5) | NM_001171932 |
| Cdh23, Cadherin related 23 (variant 6) | NM_001171933 |
| Cdh23, Cadherin related 23 (variant 7) | NM_001171934 |
| Cdh23, Cadherin related 23 (variant 8) | NM_001171935 |
| Cdh23, Cadherin related 23 (variant 9) | NM_001171936 |
| Kcnq1, Potassium voltage-gated channel | NM_000218 |
| subfamily Q member 1 (variant 1) | |
| Kcnq1, Potassium voltage-gated channel | NM_181798 |
| subfamily Q member 1 (variant 2) | |
| Kcne1, Potassium voltage-gated channel | NM_001127670 |
| subfamily E regulatory subunit 1 (variant 1) | |
| Kcne1, Potassium voltage-gated channel | NM_000219 |
| subfamily E regulatory subunit 1 (variant 2) | |
| Kcne1, Potassium voltage-gated channel | NM_001127668 |
| subfamily E regulatory subunit 1 (variant 3) | |
| Kcne1, Potassium voltage-gated channel | NM_001127669 |
| subfamily E regulatory subunit 1 (variant 4) | |
| Kcne1, Potassium voltage-gated channel | NM_001270402 |
| subfamily E regulatory subunit 1 (variant 5) | |
| Kcne1, Potassium voltage-gated channel | NM_001270403 |
| subfamily E regulatory subunit 1 (variant 6) | |
| Kcne1, Potassium voltage-gated channel | NM_001270404 |
| subfamily E regulatory subunit 1 (variant 7) | |
| Kcne1, Potassium voltage-gated channel | NM_001270405 |
| subfamily E regulatory subunit 1 (variant 8) | |
| Col1a1, Collagen type I alpha 1 chain | NM_000088 |
| Col1a2, Collagen type I alpha 2 chain | NM_000089 |
| Col2a1, Collagen type II alpha 1 chain (variant 1) | NM_001844 |
| Col2a1, Collagen type II alpha 1 chain (variant 2) | NM_033150 |
| Col3a1, Collagen type III alpha 1 chain | NM_000090 |
| Col4a1, Collagen type IV alpha 1 chain (variant 1) | NM_001845 |
| Col4a1, Collagen type IV alpha 1 chain (variant 2) | NM_001303110 |
| Col4a2, Collagen type IV alpha 2 chain | NM_001846 |
| Col4a3, Collagen type IV alpha 3 chain | NM_000091 |
| Col4a4, Collagen type IV alpha 4 chain | NM_000092 |
| Col4a5, Collagen type IV alpha 5 chain (variant 1) | NM_000495 |
| Col4a5, Collagen type IV alpha 5 chain (variant 2) | NM_033380 |
| Col4a6, Collagen type IV alpha 6 chain (variant A) | NM_001847 |
| Col4a6, Collagen type IV alpha 6 chain (variant B) | NM_033641 |
| Col4a6, Collagen type IV alpha 6 chain (variant 3) | NM_001287758 |
| Col4a6, Collagen type IV alpha 6 chain (variant 4) | NM_001287759 |
| Col4a6, Collagen type IV alpha 6 chain (variant 5) | NM_001287760 |
| Col5a1, Collagen type V alpha 1 chain (variant 1) | NM_000093 |
| Col5a1, Collagen type V alpha 1 chain (variant 2) | NM_001278074 |
| Col5a2, Collagen type V alpha 2 chain | NM_000393 |
| Col5a3, Collagen type V alpha 3 chain | NM_015719 |
| Col6a1, Collagen type VI alpha 1 chain | NM_001848 |
| Col6a2, Collagen type VI alpha 2 chain (variant 2C2) | NM_001849 |
| Col6a2, Collagen type VI alpha 2 chain (variant 2C2a) | NM_058174 |
| Col6a2, Collagen type VI alpha 2 chain (variant 2C2a′) | NM_058175 |
| Col6a3, Collagen type VI alpha 3 chain (variant 1) | NM_004369 |
| Col6a3, Collagen type VI alpha 3 chain (variant 2) | NM_057164 |
| Col6a3, Collagen type VI alpha 3 chain (variant 3) | NM_057165 |
| Col6a3, Collagen type VI alpha 3 chain (variant 4) | NM_057166 |
| Col6a3, Collagen type VI alpha 3 chain (variant 5) | NM_057167 |
| Col6a5, Collagen type VI alpha 5 chain (variant 1) | NM_001278298 |
| Col6a5, Collagen type VI alpha 5 chain (variant 2) | NM_153264 |
| Col6a6, Collagen type VI alpha 6 chain | NM_001102608 |
| Col7a1, Collagen type VII alpha 1 chain | NM_000094 |
| Col8a1, Collagen type VIII alpha 1 chain (variant 1) | NM_001850 |
| Col8a1, Collagen type VIII alpha 1 chain (variant 2) | NM_020351 |
| Col8a2, Collagen type VIII alpha 2 chain (variant 1) | NM_005202 |
| Col8a2, Collagen type VIII alpha 2 chain (variant 2) | NM_001294347 |
| Col9a1, Collagen type IX alpha 1 chain (variant 1) | NM_001851 |
| Col9a1, Collagen type IX alpha 1 chain (variant 2) | NM_078485 |
| Col9a2, Collagen type IX alpha 2 chain | NM_001852 |
| Col9a3, Collagen type IX alpha 3 chain | NM_001853 |
| Col10a1, Collagen type X alpha 1 chain | NM_000493 |
| Col11a1, Collagen type XI alpha 1 chain (variant A) | NM_001854 |
| Col11a1, Collagen type XI alpha 1 chain (variant B) | NM_080629 |
| Col11a1, Collagen type XI alpha 1 chain (variant C) | NM_080630 |
| | NM_001168249 |
| Col11a1, Collagen type XI alpha 1 chain (variant E) | NM_001190709 |
| Col11a2, Collagen type XI alpha 2 chain (variant 1) | NM_080680 |
| Col11a2, Collagen type XI alpha 2 chain (variant 2) | NM_080681 |
| Col11a2, Collagen type XI alpha 2 chain (variant 3) | NM_080679 |
| Col11a2, Collagen type XI alpha 2 chain (variant 4) | NM_001163771 |
| Col12a1, Collagen type XII alpha 1 chain (short variant) | NM_080645 |
| Col12a1, Collagen type XII alpha 1 chain (long variant) | NM_004370 |
| Col13a1, Collagen type XIII alpha 1 chain (variant 1) | NM_001130103 |
| Col13a1, Collagen type XIII alpha 1 chain (variant 5) | NM_080801 |
| Col13a1, Collagen type XIII alpha 1 chain (variant 11) | NM_080800 |
| Col13a1, Collagen type XIII alpha 1 chain (variant 15) | NM_080802 |
| Col13a1, Collagen type XIII alpha 1 chain (variant 21) | NM_080798 |
| Col13a1, Collagen type XIII alpha 1 chain (variant 22) | NM_001320951 |
| Col14a1, Collagen type XIV alpha 1 chain | NM_021110 |
| Col15a1, Collagen type XV alpha 1 chain | NM_001855 |
| Col16a1, Collagen type XVI alpha 1 chain | NM_001856 |
| Col17a1, Collagen type XVII alpha 1 chain | NM_000494 |
| Col18a1, Collagen type XVIII alpha 1 chain (variant 1) | NM_030582 |
| Col18a1, Collagen type XVIII alpha 1 chain (variant 2) | NM_130444 |
| Col18a1, Collagen type XVIII alpha 1 chain (variant 3) | NM_130445 |
| Col19a1, Collagen type XIX alpha 1 chain | NM_001858 |
| Col20a1, Collagen type XX alpha 1 chain | NM_020882 |
| Col21a1, Collagen type XXI alpha 1 chain (variant 1) | NM_030820 |
| Col21a1, Collagen type XXI alpha 1 chain (variant 2) | NM_001318751 |
| Col21a1, Collagen type XXI alpha 1 chain (variant 3) | NM_001318752 |
| Col21a1, Collagen type XXI alpha 1 chain (variant 4) | NM_001318753 |
| Col21a1, Collagen type XXI alpha 1 chain (variant 5) | NM_001318754 |
| Col22a1, Collagen type XXII alpha 1 chain | NM_152888 |
| Col23a1, Collagen type XXIII alpha 1 chain | NM_173465 |
| Col24a1, Collagen type XXIV alpha 1 chain (variant 1) | NM_152890 |
| Col24a1, Collagen type XXIV alpha 1 chain (variant 2) | NM_001349955 |
| Col25a1, Collagen type XXV alpha 1 chain (variant 1) | NM_198721 |
| Col25a1, Collagen type XXV alpha 1 chain (variant 2) | NM_032518 |
| Col25a1, Collagen type XXV alpha 1 chain (variant 3) | NM_001256074 |
| Col26a1, Collagen type XXVI alpha 1 chain (variant 1) | NM_001278563 |
| Col26a1, Collagen type XXVI alpha 1 chain (variant 2) | NM_133457 |
| Col27a1, Collagen type XXVII alpha 1 chain | NM_032888 |
| Col28a1, Collagen type XXVIII alpha 1 chain | NM_001037763 |
| Ceacam16, Carcinoembryonic antigen related cell | NM_001039213 |
| adhesion molecule 16 | |
| Otoa, Otoancorin (variant 1) | NM_144672 |
| Otoa, Otoancorin (variant 2) | NM_170664 |
| Otoa, Otoancorin (variant 3) | NM_001161683 |
| Slc26a4, Solute carrier family 26 member 4 | NM_000441 |
| Sox9, SRY-box 9 | NM_000346 |
| Sox10, SRY-box 10 | NM_006941 |
| Sall2, Spalt like transcription factor 2 (variant 1) | NM_005407 |
| Sall2, Spalt like transcription factor 2 (variant 2) | NM_001291446 |
| Sall2, Spalt like transcription factor 2 (variant 3) | NM_001291447 |
| Sall2, Spalt like transcription factor 2 (variant 6) | NM_001364564 |
| Camta1, Calmodulin binding transcription | NM_015215 |
| activator 1 (variant 1) | |
| Camta1, Calmodulin binding transcription | NM_00119556 |
| activator 1 (variant 2) | |
| Camta1, Calmodulin binding transcription | NM_001242701 |
| activator 1 (variant 3) | |
| Camta1, Calmodulin binding transcription | NM_001349608 |
| activator 1 (variant 5) | |
| Camta1, Calmodulin binding transcription | NM_001349609 |
| activator 1 (variant 6) | |
| Camta1, Calmodulin binding transcription | NM_001349610 |
| activator 1 (variant 7) | |
| Camta1, Calmodulin binding transcription | NM_001349612 |
| activator 1 (variant 8) | |
| Camta1, Calmodulin binding transcription | NM_001349613 |
| activator 1 (variant 9) | |
| Camta1, Calmodulin binding transcription | NM_001349614 |
| activator 1 (variant 10) | |
| Camta1, Calmodulin binding transcription | NM_001349615 |
| activator 1 (variant 11) | |
| Camta1, Calmodulin binding transcription | NM_001349616 |
| activator 1 (variant 12) | |
| Camta1, Calmodulin binding transcription | NM_001349617 |
| activator 1 (variant 13) | |
| Camta1, Calmodulin binding transcription | NM_001349618 |
| activator 1 (variant 14) | |
| Camta1, Calmodulin binding transcription | NM_00134961 |
| activator 1 (variant 15) | |
| Camta1, Calmodulin binding transcription | NM_001349620 |
| activator 1 (variant 16) | |
| Camta1, Calmodulin binding transcription | NM_001349621 |
| activator 1 (variant 17) | |
| Camta1, Calmodulin binding transcription | NM_001349622 |
| activator 1 (variant 18) | |
| Camta1, Calmodulin binding transcription | NM_001349623 |
| activator 1 (variant 19) | |
| Camta1, Calmodulin binding transcription | NM_001349624 |
| activator 1 (variant 20) | |
| Camta1, Calmodulin binding transcription | NM_001349625 |
| activator 1 (variant 21) | |
| Camta1, Calmodulin binding transcription | NM_001349626 |
| activator 1 (variant 22) | |
| Camta1, Calmodulin binding transcription | NM_001349627 |
| activator 1 (variant 23) | |
| Hey1, Hes related family bHLH transcription | NM_012258 |
| factor with YRPW motif 1 (variant 1) | |
| Hey1, Hes related family bHLH transcription | NM_001040708 |
| factor with YRPW motif 1 (variant 2) | |
| Hey1, Hes related family bHLH transcription | NM_001282851 |
| factor with YRPW motif 1 (variant 3) | |
| Hey2, Hes related family bHLH transcription | NM_012259 |
| factor with YRPW motif 2 | |
| Gata2, GATA binding protein 2 (variant 1) | NM_001145661 |
| Gata2, GATA binding protein 2 (variant 2) | NM_032638 |
| Gata2, GATA binding protein 2 (variant 3) | NM_001145662 |
| Gata3, GATA binding protein 3 (variant 1) | NM_001002295 |
| Gata3, GATA binding protein 3 (variant 2) | NM_002051 |
| Lass2, Ceramide synthase 2 (variant 1) | NM_181746 |
| Lass2, Ceramide synthase 2 (variant 2) | NM_022075 |
| Cux1, Cut like homeobox 1 (variant 1) | NM_181552 |
| Cux1, Cut like homeobox 1 (variant 2) | NM_001913 |
| Cux1, Cut like homeobox 1 (variant 3) | NM_181500 |
| Cux1, Cut like homeobox 1 (variant 4) | NM_001202543 |
| Cux1, Cut like homeobox 1 (variant 5) | NM_001202544 |
| Cux1, Cut like homeobox 1 (variant 6) | NM_001202545 |
| Cux1, Cut like homeobox 1 (variant 7) | NM_001202546 |
| Nr2f1, Nuclear receptor subfamily 2 | NM_005654 |
| group F member 1 | |
| Hes1, Hes family bHLH transcription factor 1 | NM_005524 |
| Rorb, RAR related orphan receptor B (variant 1) | NM_006914 |
| Rorb, RAR related orphan receptor B (variant 2) | NM_001365023 |
| Jun, Jun proto-oncogene AP-1 transcription | NM_002228 |
| factor subunit | |
| Zfp667 (human Znf667), Zinc finger | NM_022103 |
| protein 667 (variant 1) | |
| Zfp667 (human Znf667), Zinc finger | NM_00132135 |
| protein 667 (variant 2) | |
| Zfp667 (human Znf667), Zinc finger | NM_001321355 |
| protein 667 (variant 3) | |
| Lhx3, Lim homeobox 3 (variant 1) | NM_178138 |
| Lhx3, Lim homeobox 3 (variant 2) | NM_014564 |
| Lhx3, Lim homeobox 3 (variant 3) | NM_001363746 |
| Nhlh1, Nescient helix-loop-helix 1 | NM_005598 |
| Zmiz1, Zinc finger MIZ-type containing 1 | NM_020338 |
| Myt1, Myelin transcription factor 1 | NM_004535 |
| Stat3, Signal transducer and activator of | NM_139276 |
| transcription 3 (variant 1) | |
| Stat3, Signal transducer and activator of | NM_003150 |
| transcription 3 (variant 2) | |
| Stat3, Signal transducer and activator of | NM_213662 |
| transcription 3 (variant 3) | |
| Barhl1, BarH like homeobox 1 | NM_020064 |
| Tox, Thymocyte selection associated high | NM_014729 |
| mobility group box | |
| Prox1, Prospero homeobox 1 (variant 1) | NM_001270616 |
| Prox1, Prospero homeobox 1 (variant 2) | NM_002763 |
| Nfia, Nuclear factor I A (variant 1) | NM_00113467 |
| Nfia, Nuclear factor I A (variant 2) | NM_005595 |
| Nfia, Nuclear factor I A (variant 3) | NM_001145511 |
| Nfia, Nuclear factor I A (variant 4) | NM_001145512 |
| Thrb, Thyroid hormone receptor beta (variant 1) | NM_000461 |
| Thrb, Thyroid hormone receptor beta (variant 2) | NM_001128176 |
| Thrb, Thyroid hormone receptor beta (variant 3) | NM_001128177 |
| Thrb, Thyroid hormone receptor beta (variant 4) | NM_001252634 |
| Thrb, Thyroid hormone receptor beta (variant 5) | NM_001354708 |
| Thrb, Thyroid hormone receptor beta (variant 6) | NM_001354709 |
| Thrb, Thyroid hormone receptor beta (variant 7) | NM_001354710 |
| Thrb, Thyroid hormone receptor beta (variant 8) | NM_001354711 |
| Thrb, Thyroid hormone receptor beta (variant 9) | NM_001354712 |
| Thrb, Thyroid hormone receptor beta (variant 10) | NM_001354713 |
| Thrb, Thyroid hormone receptor beta (variant 11) | NM_001354714 |
| Thrb, Thyroid hormone receptor beta (variant 12) | NM_001354715 |
| Mycl1, MYCL proto-oncogene BHLH | NM_001033081 |
| transcription factor (variant 1) | |
| Mycl1, MYCL proto-oncogene BHLH | NM_001033082 |
| transcription factor (variant 2) | |
| Mycl1, MYCL proto-oncogene BHLH | NM_005376 |
| transcription factor (variant 3) | |
| Kdm5a, Lysine demethylase 5A | NM_001042603 |
| Creb3l4, cAMP responsive element | NM_130898 |
| binding protein 3 like 4 (variant 1) | |
| Creb3l4, cAMP responsive element | NM_001255978 |
| binding protein 3 like 4 (variant 2) | |
| Creb3l4, cAMP responsive element | NM_001255979 |
| binding protein 3 like 4 (variant 3) | |
| Creb3l4, cAMP responsive element | NM_001255980 |
| binding protein 3 like 4 (variant 4) | |
| Creb3l4, cAMP responsive element | NM_001255981 |
| binding protein 3 like 4 (variant 5) | |
| Creb3l4, cAMP responsive element | NR_045658 |
| binding protein 3 like 4 (variant 6) | |
| Etv1, ETS variant 1 (variant 1) | NM_004956 |
| Etv1, ETS variant 1 (variant 2) | NM_001163147 |
| Etv1, ETS variant 1 (variant 3) | NM_001163148 |
| Etv1, ETS variant 1 (variant 4) | NM_001163149 |
| Etv1, ETS variant 1 (variant 5) | NM_001163150 |
| Etv1, ETS variant 1 (variant 6) | NM_001163151 |
| Etv1, ETS variant 1 (variant 7) | NM_001163152 |
| Peg3, Paternally expressed 3 (variant 1) | NM_006210 |
| Peg3, Paternally expressed 3 (variant 2) | NM_001146184 |
| Peg3, Paternally expressed 3 (variant 3) | NM_001146185 |
| Peg3, Paternally expressed 3 (variant 4) | NM_001146186 |
| Peg3, Paternally expressed 3 (variant 5) | NM_001146187 |
| Bach2, BTB domain and CNC | NM_021813 |
| homolog 2 (variant 1) | |
| Bach2, BTB domain and CNC | NM_001170794 |
| homolog 2 (variant 2) | |
| Zbtb38, Zinc finger and BTB domain | NM_001080412 |
| containing 38 (variant 1) | |
| Zbtb38, Zinc finger and BTB domain | NM_001350099 |
| containing 38 (variant 2) | |
| Zbtb38, Zinc finger and BTB domain | NM_001350100 |
| containing 38 (variant 3) | |
| Lbh, Limb bud and heart development | NM_030915 |
| Tub, Tubby bipartite transcription factor (variant 1) | NM_003320 |
| Tub, Tubby bipartite transcription factor (variant 2) | NM_177972 |
| Hmg20, High mobility group20A (variant 1) | NM_018200 |
| Hmg20, High mobility group20A (variant 2) | NM_001304504 |
| Hmg20, High mobility group20A (variant 3) | NM_001304505 |
| Rest, RE1 silencing transcription factor (variant 1) | NM_005612 |
| Rest, RE1 silencing transcription factor (variant 2) | NM_001193508 |
| Rest, RE1 silencing transcription factor (variant 3) | NM_001363453 |
| Zfp827 (human Znf827;), Zinc finger | NM_001306215 |
| protein 827 (variant 1) | |
| Zfp827 (human Znf827;), Zinc finger | NM_178835 |
| protein 827 (variant 2) | |
| Aff3, AFR/FMR2 family member 3 (variant 1) | NM_002285 |
| Aff3, AFR/FMR2 family member 3 (variant 2) | NM_001025108 |
| Pknox2, PBX/knotted homeobox 2 | NM_022062 |
| Arid3b, AT-rich interaction domain 3B (variant 1) | NM_001307939 |
| Arid3b, AT-rich interaction domain 3B (variant 2) | NM_006465 |
| Mixip, MLX interacting protein | NM_014938 |
| Zfp532 (human Znf532), Zinc finger | NM_018181 |
| protein 532 (variant 1) | |
| Zfp532 (human Znf532), Zinc finger | NM_001318726 |
| protein 532 (variant 2) | |
| Zfp532 (human Znf532), Zinc finger | NM_001318727 |
| protein 532 (variant 3) | |
| Zfp532 (human Znf532), Zinc finger | NM_001318728 |
| protein 532 (variant 4) | |
| Zfp532 (human Znf532), Zinc finger | NM_001353525 |
| protein 532 (variant 5) | |
| Zfp532 (human Znf532), Zinc finger | NM_001353526 |
| protein 532 (variant 6) | |
| Zfp532 (human Znf532), Zinc finger | NM_001353527 |
| protein 532 (variant 7) | |
| Zfp532 (human Znf532), Zinc finger | NM_001353528 |
| protein 532 (variant 8) | |
| Zfp532 (human Znf532), Zinc finger | NM_001353529 |
| protein 532 (variant 9) | |
| Zfp532 (human Znf532), Zinc finger | NM_001353530 |
| protein 532 (variant 10) | |
| Zfp532 (human Znf532), Zinc finger | NM_001353531 |
| protein 532 (variant 11) | |
| Zfp532 (human Znf532), Zinc finger | NM_001353532 |
| protein 532 (variant 12) | |
| Zfp532 (human Znf532), Zinc finger | NM_001353533 |
| protein 532 (variant 13) | |
| Zfp532 (human Znf532), Zinc finger | NM_001353534 |
| protein 532 (variant 14) | |
| Zfp532 (human Znf532), Zinc finger | NM_001353535 |
| protein 532 (variant 15) | |
| Zfp532 (human Znf532), Zinc finger | NM_001353536 |
| protein 532 (variant 16) | |
| Zfp532 (human Znf532), Zinc finger | NM_001353537 |
| protein 532 (variant 17) | |
| Zfp532 (human Znf532), Zinc finger | NM_001353538 |
| protein 532 (variant 18) | |
| Ikzf2, IKAROS family zinc finger 2 (variant 1) | NM_016260 |
| Ikzf2, IKAROS family zinc finger 2 (variant 2) | NM_001079526 |
| Sall1, Spalt like transcription factor 1 (variant 1) | NM_00296 |
| Sall1, Spalt like transcription factor 1 (variant 2) | NM_001127892 |
| Six2, SIX homeobox 2 | NM_016932 |
| Sall3, Spalt like transcription factor 3 | NM_171999 |
| Lin28b, Lin-28 homolog B | NM_001004317 |
| Rfx7, Regulatory factor X7 | NM_022841 |
| Sox4, SRY-box 4 | NM_003107 |
| Bdnf, Brain derived neurotrophic factor (variant 1) | NM_170735 |
| Bdnf, Brain derived neurotrophic factor (variant 2) | NM_170732 |
| Bdnf, Brain derived neurotrophic factor (variant 3) | NM_170731 |
| Bdnf, Brain derived neurotrophic factor (variant 4) | NM_001709 |
| Bdnf, Brain derived neurotrophic factor (variant 5) | NM_17073 |
| Bdnf, Brain derived neurotrophic factor (variant 6) | NM_170734 |
| Bdnf, Brain derived neurotrophic factor (variant 7) | NM_001143805 |
| Bdnf, Brain derived neurotrophic factor (variant 8) | NM_001143806 |
| Bdnf, Brain derived neurotrophic factor (variant 9) | NM_001143807 |
| Bdnf, Brain derived neurotrophic factor (variant 10) | NM_001143808 |
| Bdnf, Brain derived neurotrophic factor (variant 11) | NM_001143811 |
| Bdnf, Brain derived neurotrophic factor (variant 12) | NM_001143812 |
| Bdnf, Brain derived neurotrophic factor (variant 13) | NM_001143813 |
| Bdnf, Brain derived neurotrophic factor (variant 14) | NM_001143814 |
| Bdnf, Brain derived neurotrophic factor (variant 16) | NM_001143816 |
| Bdnf, Brain derived neurotrophic factor (variant 17) | NM_001143809 |
| Bdnf, Brain derived neurotrophic factor (variant 18) | NM_001143810 |
| Ntf3, Neurotrophin 3 (variant 1) | NM_001102654 |
| Ntf3, Neurotrophin 3 (variant 2) | NM_002527 |
| Sox11, SRY-box 11 | NM_003108 |
| Tecta, Tectorin alpha | NM_005422 |
| Tectb, Tectorin beta | NM_058222 |
| Gjb2, Gap junction protein beta 2 | NM_004004 |
| Gjb6, Gap junction protein beta 6 (variant 1) | NM_001110219 |
| Gjb6, Gap junction protein beta 6 (variant 2) | NM_001110220 |
| Gjb6, Gap junction protein beta 6 (variant 3) | NM_006783 |
| Gjb6, Gap junction protein beta 6 (variant 4) | NM_001110221 |
| Tmem16a, Transmembrane protein 16A | NM_018043 |
| Ngfr, Nerve growth factor receptor | NM_002507 |
| Pmp22, peripheral myelin protein 22 (variant 1) | NM_000304 |
| Pmp22, peripheral myelin protein 22 (variant 2) | NM_153321 |
| Pmp22, peripheral myelin protein 22 (variant 3) | NM_153322 |
| Pmp22, peripheral myelin protein 22 (variant 4) | NM_001281455 |
| Pmp22, peripheral myelin protein 22 (variant 5) | NM_001281456 |
| Pmp22, peripheral myelin protein 22 (variant 8) | NM_001330143 |
| Mpz, Myelin protein zero | NM_000530 |
| | NM_001315491 |
| Mxd4, Max dimerization protein 4 | NM_006454 |
| miR-183, microRNA 183 | NR_029615 |
| miR-96, microRNA 96 | NR_029512 |
| miR-182, microRNA 182 | NR_029614 |

TABLE 3
Amino acid sequences of Atoh1 variants

| Variant | Amino acid sequence |
|---|---|
| Atoh1 variant | MSRLLHAEEWAEVKELGDHHRQPQPHHLPQPPPPPQPPATLQAREHPVYPPEL |
| S328A amino | SLLDSTDPRAWLAPTLQGICTARAAQYLLHSPELGASEAAAPRDEVDGRGELV |
| acid sequence | RRSSGGASSSKSPGPVKVREQLCKLKGGVVVDELGCSRQRAPSSKQVNGVQKQ |
| | RRLAANARERRRMHGLNHAFDQLRNVIPSFNNDKKLSKYETLQMAQIYINALS |
| | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENAKTSPRSHRSDGEFSPHSHYSDSDEAS (SEQ ID NO: 1) |
| Atoh1 variant | MSRLLHAEEWAEVKELGDHHRQPQPHHLPQPPPPPQPPATLQAREHPVYPPEL |
| S331A | SLLDSTDPRAWLAPTLQGICTARAAQYLLHSPELGASEAAAPRDEVDGRGELV |
| amino acid | RRSSGGASSSKSPGPVKVREQLCKLKGGVVVDELGCSRQRAPSSKQVNGVQKQ |
| sequence | RRLAANARERRRMHGLNHAFDQLRNVIPSFNNDKKLSKYETLQMAQIYINALS |
| | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENSKTAPRSHRSDGEFSPHSHYSDSDEAS (SEQ ID NO: 2) |
| Atoh1 variant | MSRLLHAEEWAEVKELGDHHRQPQPHHLPQPPPPPQPPATLQAREHPVYPPEL |
| S334A | SLLDSTDPRAWLAPTLQGICTARAAQYLLHSPELGASEAAAPRDEVDGRGELV |
| amino acid | RRSSGGASSSKSPGPVKVREQLCKLKGGVVVDELGCSRQRAPSSKQVNGVQKQ |
| sequence | RRLAANARERRRMHGLNHAFDQLRNVIPSFNNDKKLSKYETLQMAQIYINALS |
| | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENSKTSPRAHRSDGEFSPHSHYSDSDEAS (SEQ ID NO: 3) |
| Atoh1 variant | MSRLLHAEEWAEVKELGDHHRQPQPHHLPQPPPPPQPPATLQAREHPVYPPEL |
| S328A/S331A | SLLDSTDPRAWLAPTLQGICTARAAQYLLHSPELGASEAAAPRDEVDGRGELV |
| amino acid | RRSSGGASSSKSPGPVKVREQLCKLKGGVVVDELGCSRQRAPSSKQVNGVQKQ |
| sequence | RRLAANARERRRMHGLNHAFDQLRNVIPSFNNDKKLSKYETLQMAQIYINALS |
| | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENAKTAPRSHRSDGEFSPHSHYSDSDEAS (SEQ ID NO: 4) |
| Atoh1 variant | MSRLLHAEEWAEVKELGDHHRQPQPHHLPQPPPPPQPPATLQAREHPVYPPEL |
| S328A/S334A | SLLDSTDPRAWLAPTLQGICTARAAQYLLHSPELGASEAAAPRDEVDGRGELV |
| amino acid | RRSSGGASSSKSPGPVKVREQLCKLKGGVVVDELGCSRQRAPSSKQVNGVQKQ |
| sequence | RRLAANARERRRMHGLNHAFDQLRNVIPSFNNDKKLSKYETLQMAQIYINALS |
| | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENAKTSPRAHRSDGEFSPHSHYSDSDEAS (SEQ ID NO: 5) |
| Atoh1 variant | MSRLLHAEEWAEVKELGDHHRQPQPHHLPQPPPPPQPPATLQAREHPVYPPEL |
| S331A/S334A | SLLDSTDPRAWLAPTLQGICTARAAQYLLHSPELGASEAAAPRDEVDGRGELV |
| amino acid | RRSSGGASSSKSPGPVKVREQLCKLKGGVVVDELGCSRQRAPSSKQVNGVQKQ |
| sequence | RRLAANARERRRMHGLNHAFDQLRNVIPSFNNDKKLSKYETLQMAQIYINALS |
| | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENSKTAPRAHRSDGEFSPHSHYSDSDEAS (SEQ ID NO: 6) |
| Atoh1 variant | MSRLLHAEEWAEVKELGDHHRQPQPHHLPQPPPPPQPPATLQAREHPVYPPEL |
| S328A/S331A/ | SLLDSTDPRAWLAPTLQGICTARAAQYLLHSPELGASEAAAPRDEVDGRGELV |
| S334 | RRSSGGASSSKSPGPVKVREQLCKLKGGVVVDELGCSRQRAPSSKQVNGVQKQ |
| amino acid | RRLAANARERRRMHGLNHAFDQLRNVIPSFNNDKKLSKYETLQMAQIYINALS |
| sequence | ELLQTPSGGEQPPPPPASCKSDHHHLRTAASYEGGAGNATAAGAQQASGGSQR |
| | PTPPGSCRTRFSAPASAGGYSVQLDALHFSTFEDSALTAMMAQKNLSPSLPGS |
| | ILQPVQEENAKTAPRAHRSDGEFSPHSHYSDSDEAS (SEQ ID NO: 7) |

In some embodiments, the polynucleotide is or encodes a component of a gene editing system. An AAV vector containing a component of a gene editing system can be operably linked to a ubiquitous promoter or a cell-specific promoter (e.g., immune cell-specific promoter). For example, the component of a gene editing system can be used to introduce an alteration (e.g., insertion, deletion (e.g., knockout), translocation, inversion, single point mutation, or other mutation) in a gene known to regulate immune cell function, such as a gene that is implicated in inner ear inflammatory or immune toxicity. Exemplary gene editing systems include zinc finger nucleases (ZFNs), Transcription Activator-Like Effector-based Nucleases (TALENs), and the clustered regulatory interspaced short palindromic repeat (CRISPR) system. ZFNs, TALENs, and CRISPR-based methods are described, e.g., in Gaj et al., Trends Biotechnol. 31:397-405, 2013.

CRISPR refers to a set of (or system including a set of) clustered regularly interspaced short palindromic repeats. A CRISPR system refers to a system derived from CRISPR and Cas (a CRISPR-associated protein) or another nuclease that can be used to silence or mutate a gene described herein. The CRISPR system is a naturally occurring system found in bacterial and archaeal genomes. The CRISPR locus is made up of alternating repeat and spacer sequences. In naturally occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically designed CRISPR and one or more appropriate Cas proteins. The CRISPR locus is transcribed into RNA and processed by Cas proteins into small RNAs that comprise a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341:833, 2013. In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a gene known to regulate inner ear cell function, such as a gene that is implicated in sensorineural hearing loss or vestibular dysfunction.

In some embodiments, the polynucleotide includes a guide RNA (gRNA) for use in a clustered regulatory interspaced short palindromic repeat (CRISPR) system for gene editing. In some embodiments, the polynucleotide includes or encodes a zinc finger nuclease (ZFN), or an mRNA encoding a ZFN, that targets (e.g., cleaves) a nucleic acid sequence (e.g., DNA sequence) of a gene known to regulate inner ear cell function, such as a gene that is implicated in sensorineural hearing loss or vestibular dysfunction. In some embodiments, the polynucleotide includes or encodes a TALEN, or an mRNA encoding a TALEN, that targets (e.g., cleaves) a nucleic acid sequence (e.g., DNA sequence) of a gene known to regulate inner ear cell function, such as a gene that is implicated in sensorineural hearing loss or vestibular dysfunction.

For example, the gRNA can be used in a CRISPR system to engineer an alteration in a gene (e.g., a gene known to regulate inner ear cell function, such as a gene that is implicated in sensorineural hearing loss or vestibular dysfunction, or an immune cell gene). In other examples, the ZFN and/or TALEN can be used to engineer an alteration in a gene (e.g., a gene known to regulate inner ear cell function, such as a gene that is implicated in sensorineural hearing loss or vestibular dysfunction, or an immune cell gene). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) an immune cell gene, e.g., the alteration is a negative regulator of function. In yet another example, the alteration corrects a defect (e.g., a mutation causing a defect), in a gene known to regulate inner ear cell function, such as a gene that is implicated in sensorineural hearing loss or vestibular dysfunction.

In certain embodiments, the CRISPR system is used to edit (e.g., to add or delete a base pair) a target gene, e.g., a gene known to regulate inner ear cell function, such as a gene that is implicated in sensorineural hearing loss or vestibular dysfunction, or an immune cell gene. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In some embodiments, the CRISPR system is used to direct Cas to a promoter of a target gene, e.g., an immune cell gene, thereby blocking an RNA polymerase sterically.

In some embodiments, a CRISPR system can be generated to edit a gene known to regulate inner ear cell or immune cell function, such as a gene that is implicated in sensorineural hearing loss, vestibular dysfunction, or immune toxicity of the inner ear using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., an immune cell gene. In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene known to regulate inner ear cell function, such as a gene that is implicated in sensorineural hearing loss or vestibular dysfunction, or an immune cell gene. In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Biol. 17:5, 2016, incorporated herein by reference.

### Pharmaceutical Compositions

The nucleic acid vectors (e.g., AAV vectors) described herein may be incorporated into a vehicle for administration into a patient, such as a human patient suffering from sensorineural hearing loss, tinnitus, or vestibular dysfunction, as is described herein. Pharmaceutical compositions containing vectors, such as viral vectors, that contain a polynucleotide encoding or containing a therapeutic agent (e.g., an inner ear protein, peptide, antibody or antigen-binding fragment thereof, RNAi sequence, miRNA, or a nuclease) or a portion thereof operably linked to a ubiquitous promoter can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Mixtures of the nucleic acid vectors (e.g., AAV vectors) with additional nucleic acid vectors or small molecule agents, peptides, antibodies or antigen-binding fragments thereof, or naked nucleic acid molecules described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the subject being treated. For local administration to the inner ear, the composition may be formulated to contain a synthetic perilymph solution. An exemplary synthetic perilymph solution includes 20-200 mM NaCl, 1-5 mM KCl, 0.1-10 mM CaCl₂), 1-10 mM glucose, and 2-50 mM HEPEs, with a pH between about 6 and 9 and an osmolality of about 300 mOsm/kg. The person responsible for administration will, in any event, determine the appropriate dose for the individual subject. Moreover, for human administration, preparations may meet sterility, pyrogenicity, general safety, and purity standards as required by FDA Office of Biologics standards.

### Methods of Treatment

The compositions described herein may be administered to a subject with sensorineural hearing loss, tinnitus, or vestibular dysfunction by a variety of routes, such as local administration to the inner ear (e.g., administration into the perilymph or endolymph, e.g., by injection or catheter insertion through the round window membrane, injection into a semicircular canal, by canalostomy, or by intratympanic or transtympanic injection, e.g., administration to an inner ear cell), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. If the compositions are administered by direct delivery to the inner ear, a second fenestration or vent hole may be added elsewhere in the inner ear. The most suitable route for administration in any given case will depend on the particular composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, monthly, or bi-weekly). In embodiments in which the therapeutic agent is expressed using a dual vector system, the first and second nucleic acid vectors of the dual vector (e.g., AAV dual vector) system can be administered simultaneously (e.g., in one composition) or sequentially (e.g., the second nucleic acid vector is administered 15 minutes, 20 minutes, 25 minutes, 30 minutes, 45 minutes, 1 hour, 2 hours, 8 hours, 12 hours, 1 day, 2 days, 7 days, two weeks, 1 month or more after the first nucleic acid vector). The first and second nucleic acid vector can have the same serotype or different serotypes (e.g., AAV serotypes).

Subjects that may be treated as described herein are subjects having or at risk of developing hearing loss (e.g., sensorineural hearing loss), tinnitus, and/or vestibular dysfunction (e.g., dizziness, vertigo, imbalance, bilateral vestibulopathy, oscillopsia, or a balance disorder). The compositions and methods described herein can be used to treat subjects having or at risk of developing damage to cochlear hair cells (e.g., damage related to acoustic trauma, disease or infection, head trauma, ototoxic drugs, or aging), subjects having or at risk of developing damage to vestibular hair cells (e.g., damage related to disease or infection, head trauma, ototoxic drugs, or aging), subjects having or at risk of developing sensorineural hearing loss, deafness, or auditory neuropathy, subjects having or at risk of developing vestibular dysfunction (e.g., dizziness, vertigo, imbalance, bilateral vestibulopathy, bilateral vestibular hypofunction, oscillopsia, or a balance disorder), subjects having tinnitus (e.g., tinnitus alone, or tinnitus that is associated with sensorineural hearing loss or vestibular dysfunction), subjects having a genetic mutation associated with hearing loss and/or vestibular dysfunction, or subjects with a family history of hereditary hearing loss, deafness, auditory neuropathy, tinnitus, or vestibular dysfunction. In some embodiments, the disease associated with damage to or loss of hair cells (e.g., cochlear and/or vestibular hair cells) is an autoimmune disease or condition in which an autoimmune response contributes to hair cell damage or death. Autoimmune diseases linked to sensorineural hearing loss and vestibular dysfunction include autoimmune inner ear disease (AIED), polyarteritis nodosa (PAN), Cogan's syndrome, relapsing polychondritis, systemic lupus erythematosus (SLE), Wegener's granulomatosis, Sjögren's syndrome, and Behçet's disease. Some infectious conditions, such as Lyme disease and syphilis can also cause hearing loss and vestibular dysfunction (e.g., by triggering autoantibody production). Viral infections, such as rubella, cytomegalovirus (CMV), lymphocytic choriomeningitis virus (LCMV), HSV types 1 &2, West Nile virus (WNV), human immunodeficiency virus (HIV) varicella zoster virus (VZV), measles, and mumps, can also cause hearing loss and vestibular dysfunction. In some embodiments, the subject has or is at risk of developing hearing loss and/or vestibular dysfunction that is associated with or results from loss of hair cells (e.g., cochlear or vestibular hair cells).

The methods described herein may include a step of screening a subject for one or more mutations in genes known to be associated with hearing loss and/or vestibular dysfunction prior to treatment with or administration of the compositions described herein. A subject can be screened for a genetic mutation using standard methods known to those of skill in the art (e.g., genetic testing). The methods described herein may also include a step of assessing hearing or vestibular function in a subject prior to treatment with or administration of the compositions described herein. Hearing can be assessed using standard tests, such as audiometry, ABR, electrocochleography (ECOG), and otoacoustic emissions. The methods described herein may also include a step of assessing vestibular function in a subject prior to treatment with or administration of the compositions described herein. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010). These tests can also be used to assess hearing and/or vestibular function in a subject after treatment with or administration of the compositions described herein. The compositions and methods described herein may also be administered as a preventative treatment to patients at risk of developing hearing loss (e.g., sensorineural hearing loss), tinnitus, or vestibular dysfunction, e.g., patients who have a family history of inherited hearing loss, tinnitus, or vestibular dysfunction, patients carrying a mutation in a gene associated with hearing loss, tinnitus, or vestibular dysfunction who do not yet exhibit hearing loss, tinnitus, or vestibular dysfunction, or patients exposed to risk factors for acquired hearing loss (e.g., acoustic trauma, disease or infection, head trauma, ototoxic drugs, or aging) or vestibular dysfunction (e.g., disease or infection, head trauma, ototoxic drugs, or aging).

Treatment may include administration of a composition containing the nucleic acid vectors (e.g., AAV vectors) described herein in various unit doses. Each unit dose will ordinarily contain a predetermined quantity of the therapeutic composition. The quantity to be administered, and the particular route of administration and formulation, are within the skill of those in the clinical arts. A unit dose need not be administered as a single injection but may include continuous infusion over a set period of time. Dosing may be performed using a syringe pump to control infusion rate in order to minimize damage to the cochlea. In cases in which the nucleic acid vectors are AAV vectors (e.g., AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S vectors), the AAV vectors may have a titer of, for example, from about 1×10⁹ vector genomes (VG)/mL to about 1×10¹⁶ VG/mL (e.g., 1×10⁹ VG/mL, 2×10⁹ VG/mL, 3×10⁹ VG/mL, 4×10⁹ VG/mL, 5×10⁹ VG/mL, 6×10⁹ VG/mL, 7×10⁹ VG/mL, 8×10⁹ VG/mL, 9×10⁹ VG/mL, 1×10¹⁰ VG/mL, 2×10¹⁰ VG/mL, 3×10¹⁰ VG/mL, 4×10¹⁰ VG/mL, 5×10¹⁰ VG/mL, 6×10¹⁰ VG/mL, 7×10¹⁰ VG/mL, 8×10¹⁰ VG/mL, 9×10¹⁰ VG/mL, 1×10¹¹ VG/mL, 2×10¹¹ VG/mL, 3×10¹¹ VG/mL, 4×10¹¹ VG/mL, 5×10¹¹ VG/mL, 6×10¹¹ VG/mL, 7×10¹¹ VG/mL, 8×10¹¹ VG/mL, 9×10¹¹ VG/mL, 1×10¹² VG/mL, 2×10¹² VG/mL, 3×10¹² VG/mL, 4×10¹² VG/mL, 5×10¹² VG/mL, 6×10¹² VG/mL, 7×10¹² VG/mL, 8×10¹² VG/mL, 9×10¹² VG/mL, 1×10¹³ VG/mL, 2×10¹³ VG/mL, 3×10¹³ VG/mL, 4×10¹³ VG/mL, 5×10¹³ VG/mL, 6×10¹³ VG/mL, 7×10¹³ VG/mL, 8×10¹³ VG/mL, 9×10¹³ VG/mL, 1×10¹⁴ VG/mL, 2×10¹⁴ VG/mL, 3×10¹⁴ VG/mL, 4×10¹⁴ VG/mL, 5×10¹⁴ VG/mL, 6×10¹⁴ VG/mL, 7×10¹⁴ VG/mL, 8×10¹⁴ VG/mL, 9×10¹⁴ VG/mL, 1×10¹⁵ VG/mL, 2×10¹⁵ VG/mL, 3×10¹⁵ VG/mL, 4×10¹⁵ VG/mL, 5×10¹⁵ VG/mL, 6×10¹⁵ VG/mL, 7×10¹⁵ VG/mL, 8×10¹⁵ VG/mL, 9×10¹⁵ VG/mL, or 1×10¹⁶ VG/mL) in a volume of 1 μL to 200 μL (e.g., 1, 2, 3, 5, 6, 7, 8, 9, 10, 15, 20, 25, 30, 35, 40, 45, 50, 55, 60, 65, 70, 75, 80, 85, 90, 95, 100, 110,120, 130, 140, 150, 160, 170, 180, 190, or 200 μL). The AAV vectors may be administered to the subject at a dose of about 1×10⁷ VG/ear to about 2×10¹⁵ VG/ear (e.g., 1×10⁷ VG/ear, 2×10⁷ VG/ear, 3×10⁷ VG/ear, 4×10⁷ VG/ear, 5×10⁷ VG/ear, 6×10⁷ VG/ear, 7×10⁷ VG/ear, 8×10⁷ VG/ear, 9×10⁷ VG/ear, 1×10⁸ VG/ear, 2×10⁸ VG/ear, 3×10⁸ VG/ear, 4×10⁸ VG/ear, 5×10⁸ VG/ear, 6×10⁸ VG/ear, 7×10⁸ VG/ear, 8×10⁸ VG/ear, 9×10⁸ VG/ear, 1×10⁹ VG/ear, 2×10⁹ VG/ear, 3×10⁹ VG/ear, 4×10⁹ VG/ear, 5×10⁹ VG/ear, 6×10⁹ VG/ear, 7×10⁹ VG/ear, 8×10⁹ VG/ear, 9×10⁹ VG/ear, 1×10¹⁰VG/ear, 2×10¹⁰ VG/ear, 3×10¹⁰ VG/ear, 4×10¹⁰ VG/ear, 5×10¹⁰ VG/ear, 6×10¹⁰ VG/ear, 7×10¹⁰ VG/ear, 8×10¹⁰ VG/ear, 9×10¹⁰ VG/ear, 1×10¹¹ VG/ear, 2×10¹¹ VG/ear, 3×10¹ VG/ear, 4×10¹ VG/ear, 5×10¹ VG/ear, 6×10¹¹ VG/ear, 7×10¹ VG/ear, 8×10¹¹ VG/ear, 9×10¹¹ VG/ear, 1×10¹² VG/ear, 2×10¹² VG/ear, 3×10¹² VG/ear, 4×10¹² VG/ear, 5×10¹² VG/ear, 6×10¹² VG/ear, 7×10¹² VG/ear, 8×10¹² VG/ear, 9×10¹² VG/ear, 1×10¹³ VG/ear, 2×10¹³ VG/ear, 3×10¹³ VG/ear, 4×10¹³ VG/ear, 5×10¹³ VG/ear, 6×10¹³ VG/ear, 7×10¹³ VG/ear, 8×10¹³ VG/ear, 9×10¹³ VG/ear, 1×10¹⁴ VG/ear, 2×10¹⁴ VG/ear, 3×10¹⁴ VG/ear, 4×10¹⁴ VG/ear, 5×10¹⁴ VG/ear, 6×10¹⁴ VG/ear, 7×10¹⁴ VG/ear, 8×10¹⁴ VG/ear, 9×10¹⁴ VG/ear, 1×10¹⁵ VG/ear, or 2×10¹⁵ VG/ear).

The compositions and methods described herein can be used to induce or increase hair cell regeneration in a subject (e.g., cochlear and/or vestibular hair cell regeneration). Subjects that may benefit from compositions that induce or increase hair cell regeneration include subjects suffering from hearing loss or vestibular dysfunction as a result of loss of hair cells (e.g., loss of hair cells related to trauma (e.g., acoustic trauma or head trauma), disease or infection, ototoxic drugs, or aging), and subjects with abnormal hair cells (e.g., hair cells that do not function properly when compared to normal hair cells), damaged hair cells (e.g., hair cell damage related to trauma (e.g., acoustic trauma or head trauma), disease or infection, ototoxic drugs, or aging), or reduced hair cell numbers due to genetic mutations or congenital abnormalities. The compositions and methods described herein can also be used to promote or increase cochlear and/or vestibular hair cell maturation, which can lead to improved hearing and/or vestibular function, respectively.

The compositions and methods described herein can also be used to prevent or reduce hearing loss and/or vestibular dysfunction caused by ototoxic drug-induced hair cell damage or death (e.g., cochlear hair cell and/or vestibular hair cell damage or death) in subjects who have been treated with ototoxic drugs, or who are currently undergoing or soon to begin treatment with ototoxic drugs. Ototoxic drugs are toxic to the cells of the inner ear, and can cause sensorineural hearing loss, vestibular dysfunction (e.g., vertigo, dizziness, imbalance, bilateral vestibulopathy, or oscillopsia), tinnitus, or a combination of these symptoms. Drugs that have been found to be ototoxic include aminoglycoside antibiotics (e.g., gentamycin, neomycin, streptomycin, tobramycin, kanamycin, vancomycin, and amikacin), viomycin, antineoplastic drugs (e.g., platinum-containing chemotherapeutic agents, such as cisplatin, carboplatin, and oxaliplatin), loop diuretics (e.g., ethacrynic acid and furosemide), salicylates (e.g., aspirin, particularly at high doses), and quinine. In some embodiments, the methods and compositions described herein can be used to treat or prevent aminoglycoside-induced bilateral vestibulopathy and oscillopsia.

The compositions described herein are administered in an amount sufficient to improve hearing, improve vestibular function (e.g., improve balance or reduce vertigo or dizziness), reduce tinnitus, treat bilateral vestibulopathy, treat oscillopsia, treat a balance disorder, increase or induce hair cell regeneration, increase hair cell numbers, or increase hair cell maturation, increase wild-type (WT) target protein expression (e.g., expression of the target protein in an inner ear hair cell, e.g., IHC, OHC, type I hair cell, or type II hair cell, or an inner ear supporting cell, e.g., vestibular supporting cell or cochlear supporting cell), increase target protein function, increase inner ear cell proliferation, or increase inner ear cell survival. Hearing may be evaluated using standard hearing tests (e.g., audiometry, ABR, electrocochleography (ECOG), and otoacoustic emissions) and may be improved by 5% or more (e.g., 5%, 10%, 15%, 20%, 30%, 40%, 50%, 60%, 70%, 80%, 90%, 100% or more) compared to hearing measurements obtained prior to treatment. In some embodiments, the compositions are administered in an amount sufficient to improve the subject's ability to understand speech. The compositions described herein may also be administered in an amount sufficient to slow or prevent the development or progression of sensorineural hearing loss or auditory neuropathy (e.g., in subjects who carry a mutation in a target protein or have a family history of autosomal recessive hearing loss but do not exhibit hearing impairment, or in subjects exhibiting mild to moderate hearing loss). In some embodiments, the compositions described herein are administered in an amount sufficient to improve the durability of hearing recovery, the magnitude of hearing recovery, durability of reduction or elimination of tinnitus, and/or magnitude of reduction of tinnitus. Vestibular function may be assessed using standard tests, such as eye movement testing (e.g., electronystagmogram (ENG) or videonystagmogram (VNG)), tests of the vestibulo-ocular reflex (VOR) (e.g., the head impulse test (Halmagyi-Curthoys test), which can be performed at the bedside or using a video-head impulse test (VHIT), or the caloric reflex test), posturography, rotary-chair testing, ECOG, vestibular evoked myogenic potentials (VEMP), and specialized clinical balance tests, such as those described in Mancini and Horak, Eur J Phys Rehabil Med, 46:239 (2010) and may be improved by 5% or more (e.g., 5%, 10%, 15%, 20%, 30%, 40%, 50%, 60%, 70%, 80%, 90%, 100% or more) compared to vestibular measurements obtained prior to treatment. In some embodiments, the compositions described herein are administered in an amount sufficient to improve the durability and/or the magnitude of recovery of vestibular function (e.g., improvements in balance or reductions in vertigo or dizziness). Protein expression may be evaluated using immunohistochemistry, Western blot analysis, quantitative real-time PCR, or other methods known in the art for detection protein or mRNA, and may be increased by 5% or more (e.g., 5%, 10%, 15%, 20%, 30%, 40%, 50%, 60%, 70%, 80%, 90%, 100% or more) compared to target protein expression prior to administration of the compositions described herein. Protein function may be evaluated directly (e.g., using electrophysiological methods or imaging methods to assess exocytosis) or indirectly based on hearing or vestibular tests, and may be increased by 5% or more (e.g., 5%, 10%, 15%, 20%, 30%, 40%, 50%, 60%, 70%, 80%, 90%, 100% or more) compared to target protein function prior to administration of the compositions described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the composition depending on the dose and route of administration used for treatment. Depending on the outcome of the evaluation, the patient may receive additional treatments.

### Kits

The compositions described herein can be provided in a kit for use in treating an inner ear dysfunction (e.g., hearing loss or vestibular dysfunction). Compositions may include nucleic acid vectors (e.g., AAV vectors) described herein, optionally packaged in an AAV virus capsid and an inhibitor of inflammatory or immune signaling described herein. The kit can further include a package insert that instructs a user of the kit, such as a physician, to perform the methods described herein. The kit may optionally include a syringe or other device for administering the composition.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1—RNA-Seq Analysis of Murine Cochlear Samples Treated with an Adeno-Associated Viral Vector Containing a Ubiquitous Promoter Compared to an Inner Hair Cell-Specific Promoter

To determine if any differential changes in gene expression occurred in in cells of the inner ear treated with an adeno-associated viral (AAV) vector containing a ubiquitous promoter versus an AAV vector containing a cell-specific promoter, the following methods were performed.

### Delivery of AAV-smCBA or AAV-Myo15 to Adult Mice

Animals were dosed with 2 μL of either a dual AAV1 vector system encoding human otoferlin (hOTOF) under the control of the ubiquitous truncated chimeric CMV-chicken β-actin promoter (smCBA promoter) (AAV-smCBA-hOTOF) or a dual AAV1 vector system encoding hOTOF under control of an inner hair cell-specific myosin 15 (Myo15; 1 kb) promoter (n=10/group) through the round window under isoflurane anesthesia. The smCBA promoter and the Myo15 promoter have been previously shown to drive similar levels of gene expression in inner hair cells (see, e.g., International Patent Application Publication No. WO2020163761 A1). Dosing concentrations were 4e10 vg/μL. Animals were allowed to recover post-operatively according to the protocol. Distortion product otoacoustic emissions (DPOAE) and auditory brainstem response (ABR) were tested 4 weeks post-treatment. Human OTOF overexpression with the smCBA promoter in wild-type animals led to elevated hearing thresholds. Animals injected with AAV-smCBA-hOTOF also exhibited loss of inner hair cells, which can explain the elevated ABR thresholds observed in these animals.

### Bulk RNA-Seq Profiling of Mouse Cochleae

Temporal bones from AAV-injected cochlea (n=5 per condition, 4 weeks after injection) were isolated in ice-cold Leibovitz's L-15 medium, the vestibular system was removed, and were flash-frozen using liquid nitrogen. RNA was extracted using the RNeasy kit (QIAGEN) and ribosomal RNA was depleted using the mouse RiboCop rRNA Depletion Kit (Lexogen). Libraries were prepared using the CORALL total RNA-seq library preparation kit (Lexogen) and evaluated using an Agilent BioAnalyzer prior to sequencing on an Illumina sequencer.

### Analysis of Mouse Cochleae Bulk RNA-Seq Data

Data was processed according to steps recommended by the CORALL hit manufacturer. In brief, UMIs were extracted from the fastq reads and appended to the read IDs, then reads were trimmed using cutadapt, aligned to a GRCm38 mm10 reference genome to which the hOTOF transgene sequence was added using STAR, PCR duplicates were removed using UMI tools, and gene expression was quantified using StringTie. Differential expression analysis between conditions was performed using DESeq2.

### Adult Mouse Whole Cochlea Single-Cell RNA-Seq

Temporal bones from AAV-injected cochleae (n=3 for each AAV-smCBA and AAV-Myo15, 4 weeks after injection) were isolated in ice-cold Leibovitz's L-15 medium, and the vestibular system was removed, leaving just the cochlea. To allow the dissociation enzymes better access to the cochlea tissue, the bone overlying the apical portion of the cochlea was removed. Micro-dissected tissue from each mouse was pooled in a solution of Leibovitz containing 200 units/mL collagenase IV (Sigma) and 10 kunitz/mL DNase I (Stem Cell Technologies) and incubated for 30 min at 37° C. to digest extracellular matrix. The collagenase IV solution was then replaced with EBSS containing 20 units of papain (Worthington Biochemical), 1 mM L-cysteine, 0.5 mM EDTA, 15 mM HEPES, and 15 kunitz/mL DNase 1 and incubated for an additional 30 minutes at 37° C., triturating with a 1000 μL pipette every 10 min to generate a single cell suspension. An equal volume of L-15 medium containing 20% ovomucoid protease inhibitor (Worthington Biochemical) was added, and the dissociated cells were passed through a 20 μm filter (PluriSelect) to remove large debris. The cells were pelleted at 350 g for 5 min, washed with PBS, and then pelleted and resuspended in PBS containing 0.1% BSA. Finally, a 10 μL sample of the cell suspension was counted on a Luna Fl automated counter using a Live/Dead assay (Thermo Fisher Scientific). The cell suspension was diluted to a concentration of ˜1,000 cells per μL and immediately captured, lysed, and primed for reverse transcription (RT) using the high throughput, droplet microfluidics Gemcode platform from 10X Genomics. Samples were sequenced on an Illumina sequencer.

### Processing and QC of Single-Cell RNA-Seq Data

Reads were demultiplexed, aligned to a GRCm38 mm10 reference genome to which the hOTOF transgene sequence was added, and filtered. Cell barcodes and UMIs were quantified using the 10X Genomics CellRanger pipeline with default parameters (software.10xgenomics.com/single-cell/overview/welcome). CellRanger uses STAR for alignment and manufacturer's software for all other steps. All further filtering and downstream analysis of single-cell data described in subsequent sections was performed with the Seurat v3 R package, using default parameters unless specified. To limit the influence of low complexity cells and genes, cells with fewer than 100 expressed genes and genes with detectable expression in 10 or fewer cells were removed. Red blood cell contamination is a concern when dissociating cells from the adult cochlea since there is a large niche of erythroid cells near the bony apex. Red blood cells are highly enriched for hemoglobin transcripts, and hemoglobin protein occupies up to 98% of the red blood cell cytosol. The fraction of total transcript counts from each single cell that were comprised of transcripts from the hemoglobin genes were analyzed (Hba-a1, Hba-a2, Hbb-bh1, Hbb-bs, Hbb-bt). Any cell with >5% contamination was removed. Cells with >25% mitochondrial or >40% ribosomal transcripts fractions were removed, as well as cells with >50,000 UMIs detected.

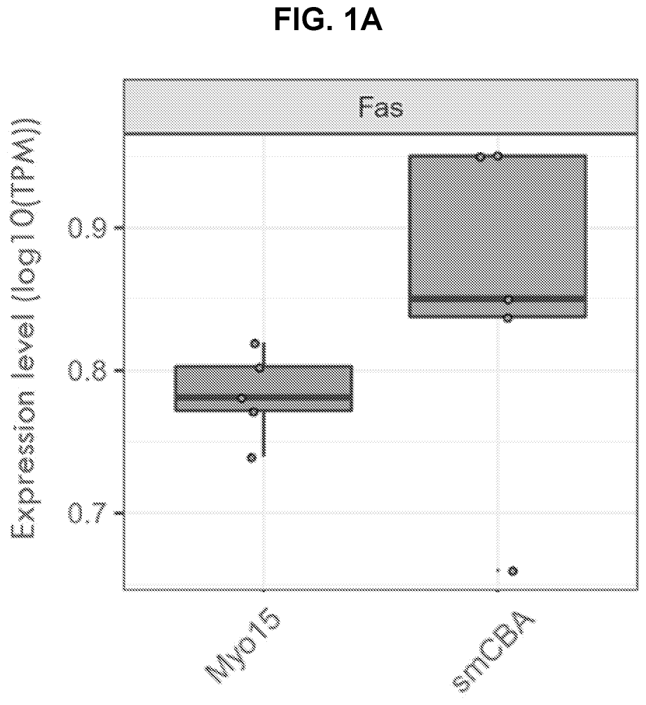

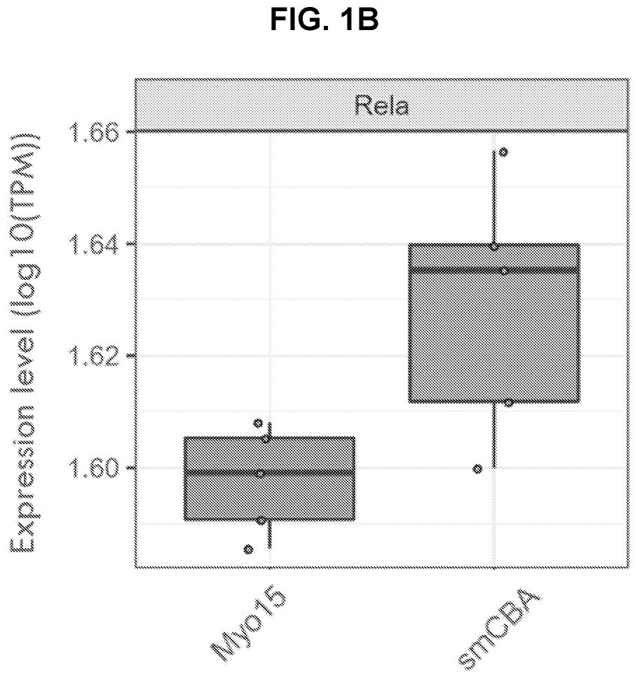

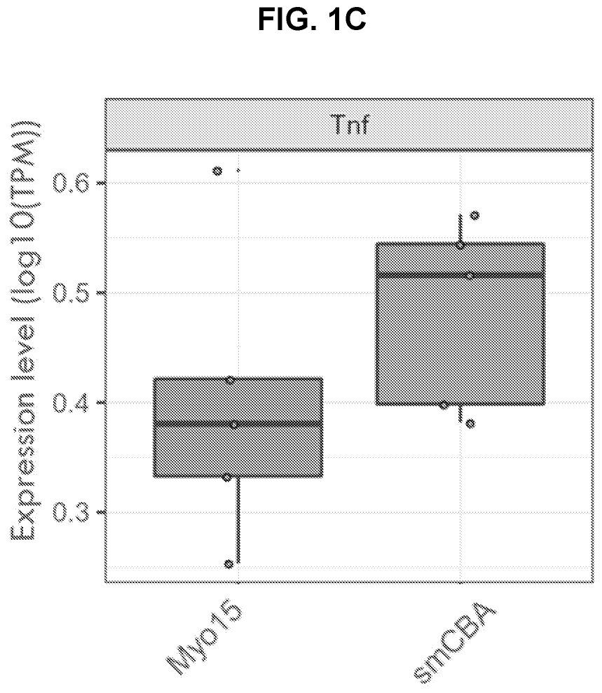

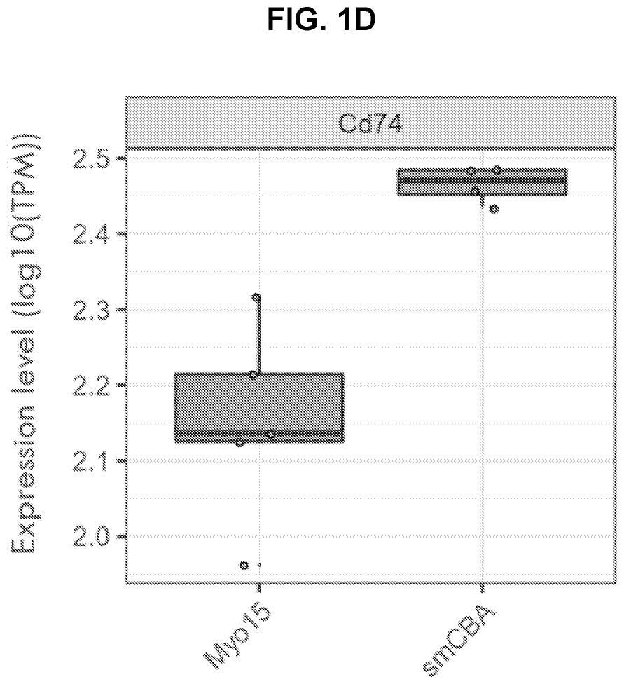

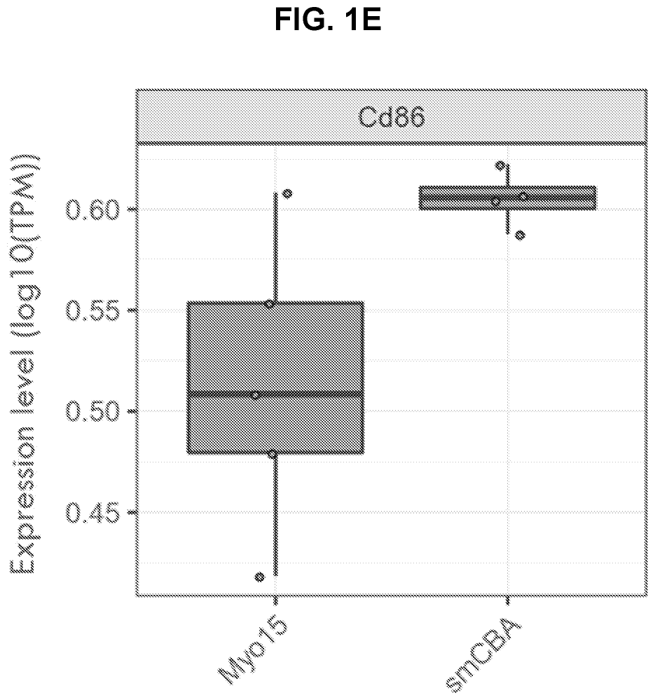

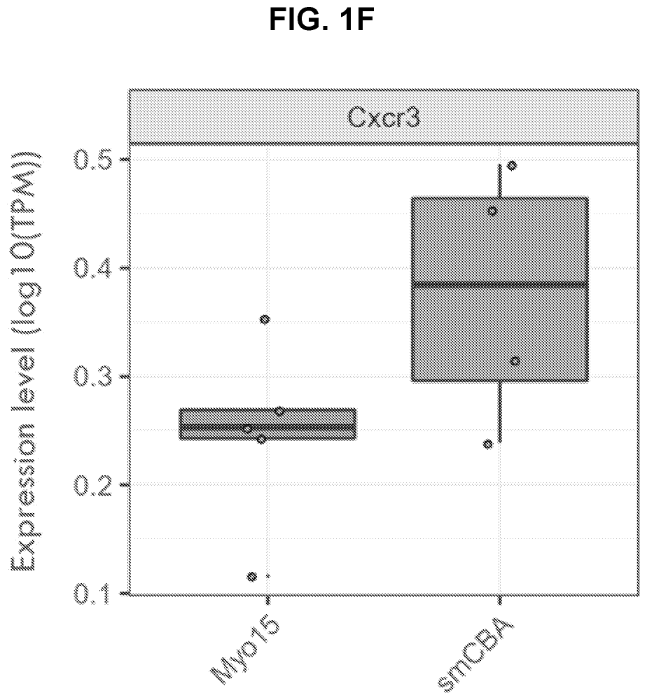

Analysis of differential gene expression between AAV-smCBA- and AAV-Myo15-treated samples revealed upregulation of genes involved in apoptosis and allograft rejection, namely Fas cell surface death receptor (Fas; FIG. 1A), RELA proto-oncogene NF-κB subunit (Rela; FIG. 1B), and tumor necrosis factor (TNF; FIG. 1C) and allograft rejection signature genes CD74 molecule (CD74; FIG. 1D), CD86 molecule (CD86; FIG. 1E), and C-X-C motif chemokine receptor 3 (CXCR3; FIG. 1F).

### Dimensionality Reduction, Clustering, Cell Classification and Differential Expression

Full descriptions of Seurat's clustering procedure can be found at satijalab.org/seurat/. Principal component analysis (PCA) was used for dimensionality reduction and was calculated from z-scored residuals of the regressed gene expression matrix. PCA was computed for the top 2,000 most highly variable genes, and the first 25 components were used for clustering and UMAP 2D projection. For cluster annotation, the expression profiles of the individual cells were compared to mean expression profiles from an adult mouse reference dataset using Spearman correlation, using the highest correlation value as the tentative cell label. Clusters were then labeled based on the majority label across individual cells. The software Scrublet was used to identify putative doublets in the dataset. Clusters with high median doublet scores were filtered out. Immune cells were further subset from the full dataset, re-clustered and re-labeled for further granularity, using the same workflow and parameters as for the full dataset. Differential expression analysis between AAV-smCBA and AAV-Myo15 cells of each cell type was performed using Seurat's FindMarkers function.

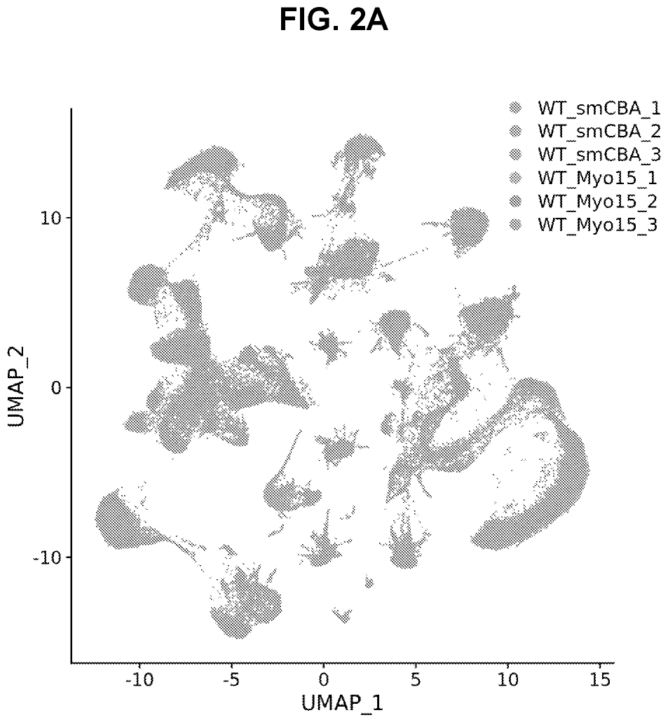

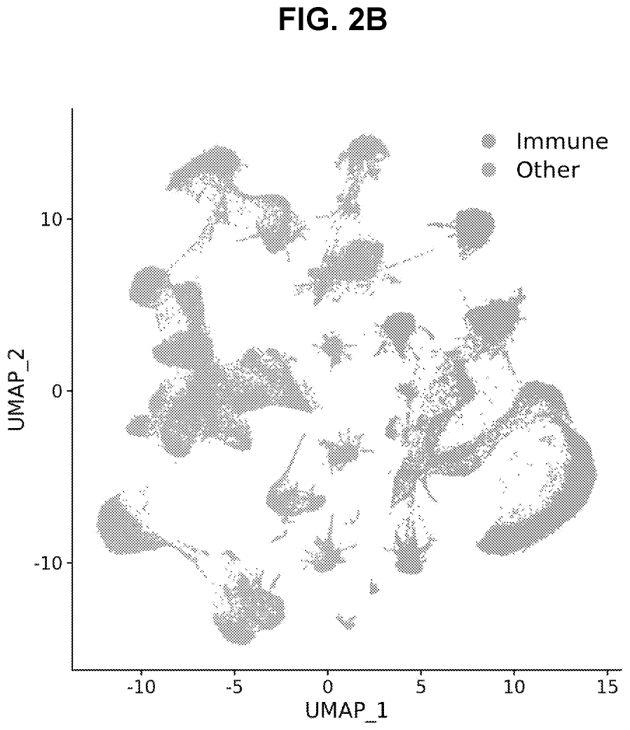

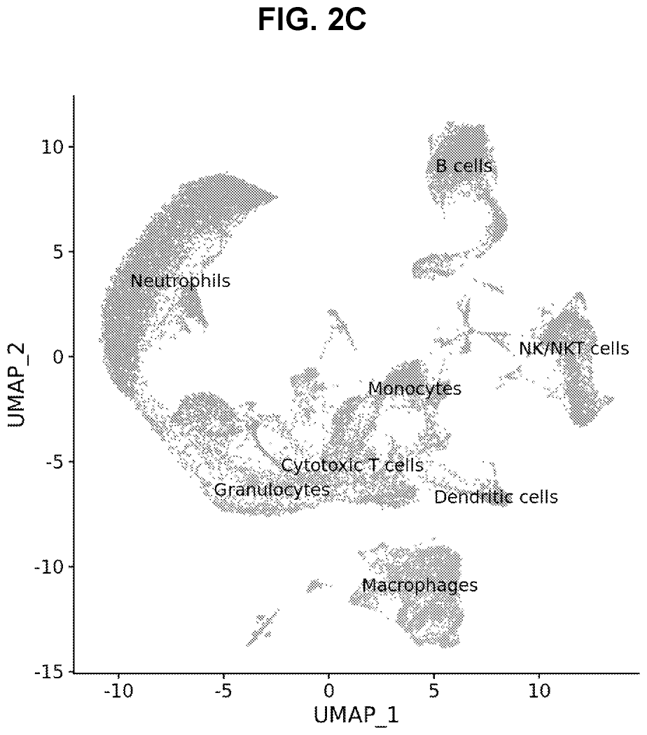

An overview of single cell RNA-seq profiling of AAV-smCBA- and AAV-Myo15-treated cochlea is shown in FIGS. 2A-2C.

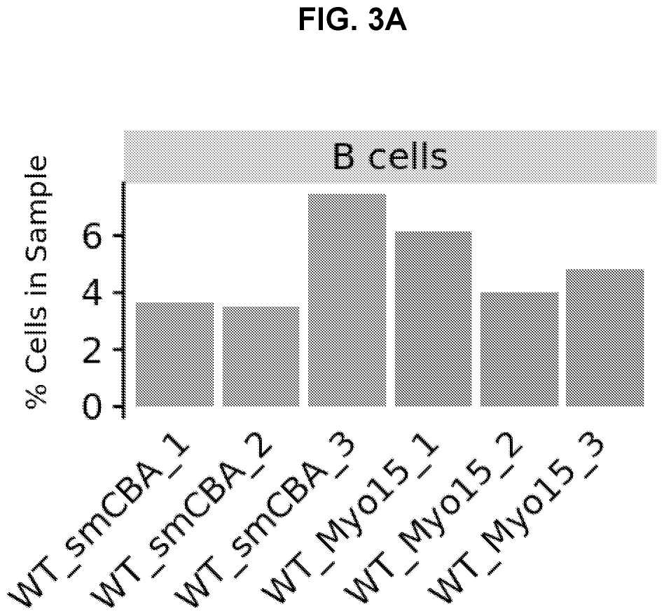

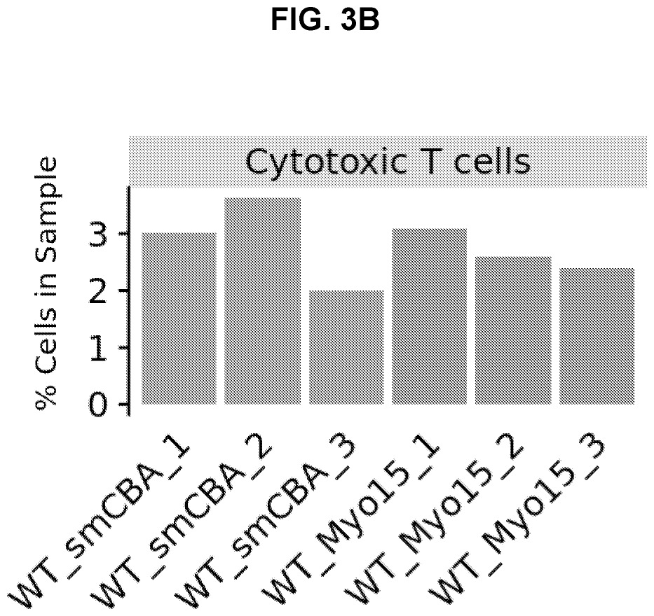

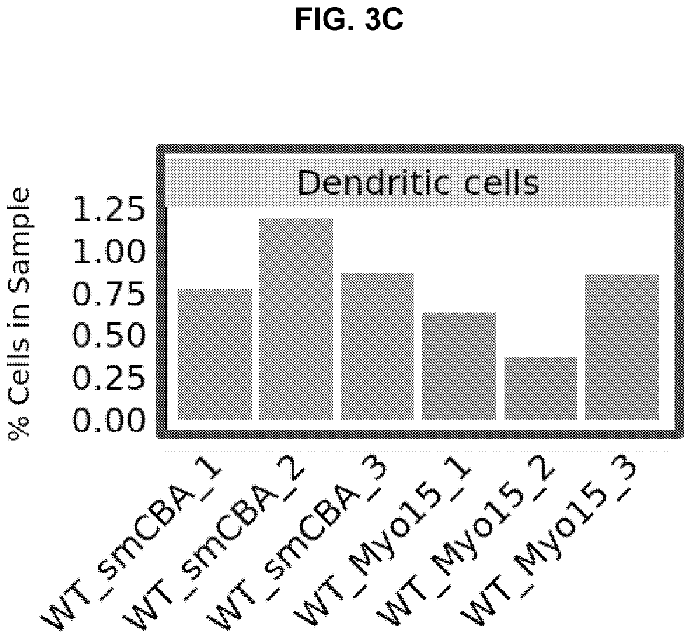

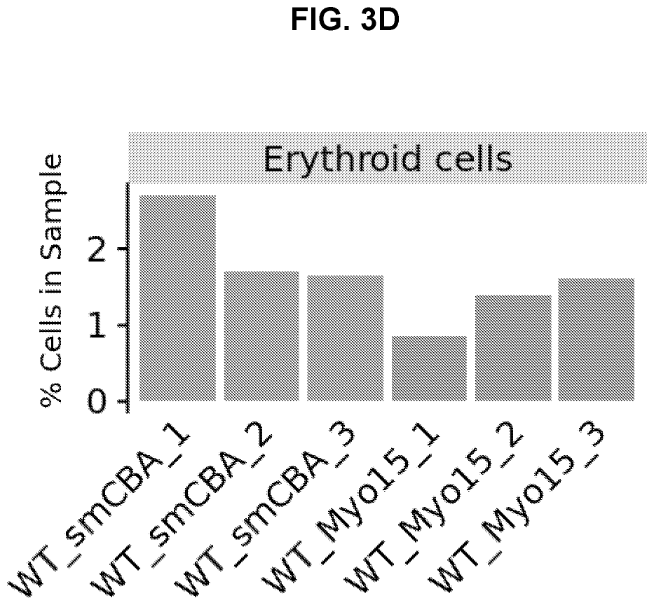

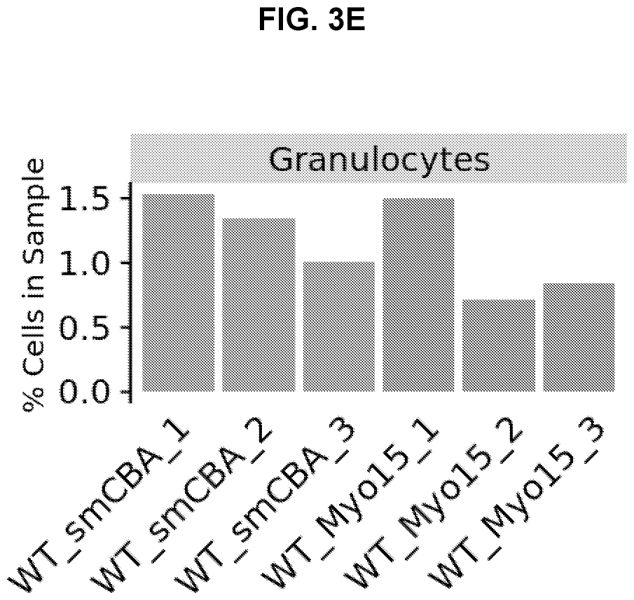

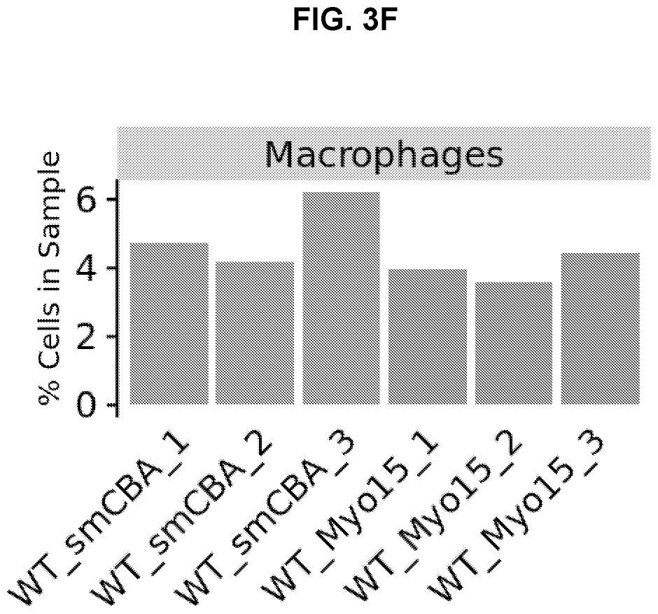

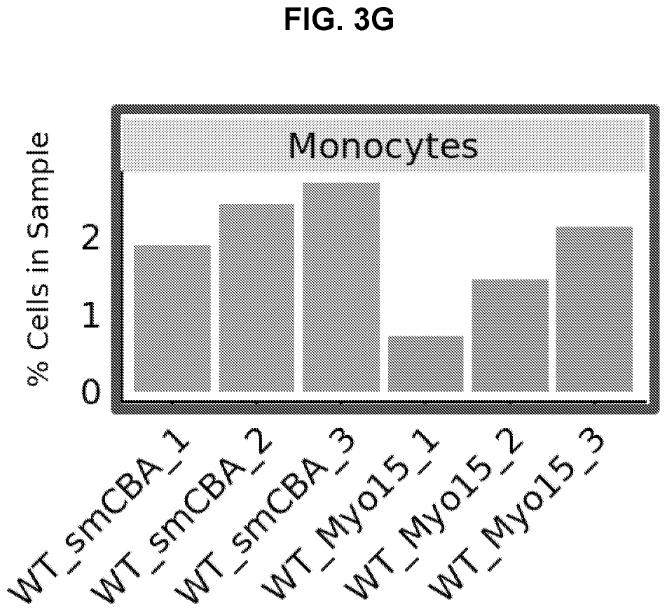

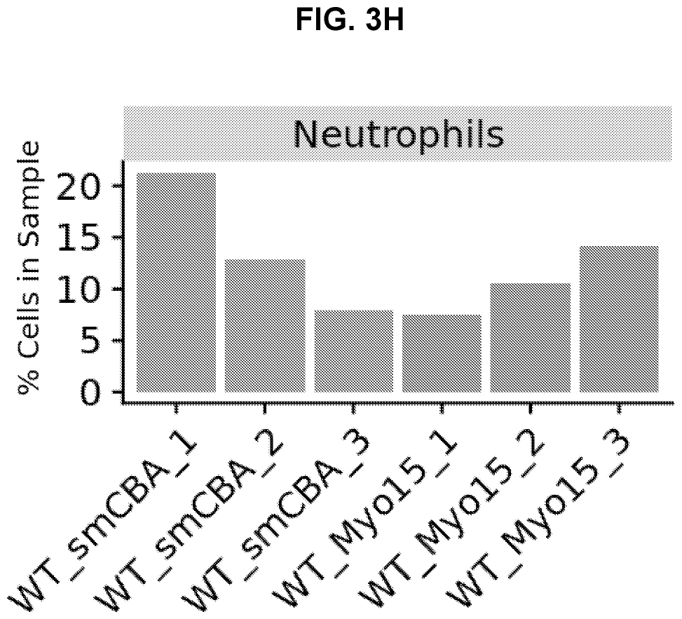

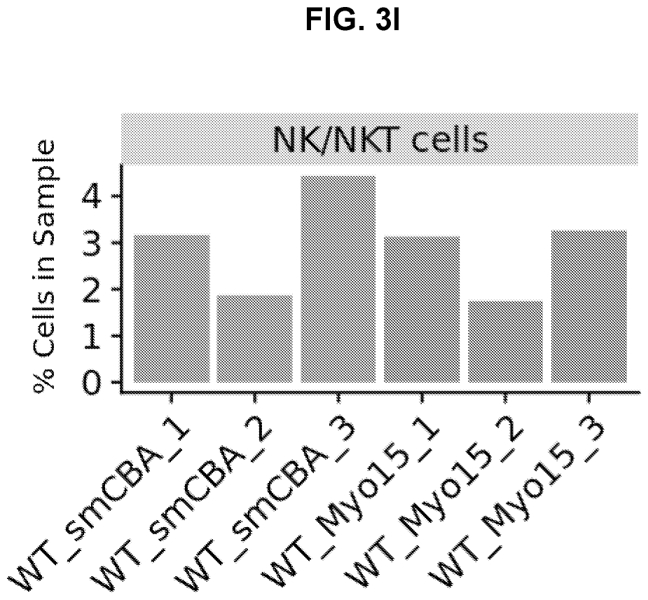

The percentages of immune cell types in AAV-smCBA- or AAV-Myo15-treated samples were analyzed based on the RNA-seq expression data described above. Analyzed cell types included B cells (FIG. 3A), cytotoxic T cells (FIG. 3B), dendritic cells (FIG. 3C), erythroid cells (FIG. 3D), granulocytes (FIG. 3E), macrophages (FIG. 3F), monocytes (FIG. 3G), neutrophils (FIG. 3H), and natural killer (NK)/natural killer T (NKT) cells (FIG. 3I). These results demonstrate an increase in the numbers of dendritic cells and monocytes in in AAV-smCBA-treated samples as compared to AAV-Myo15-treated samples.

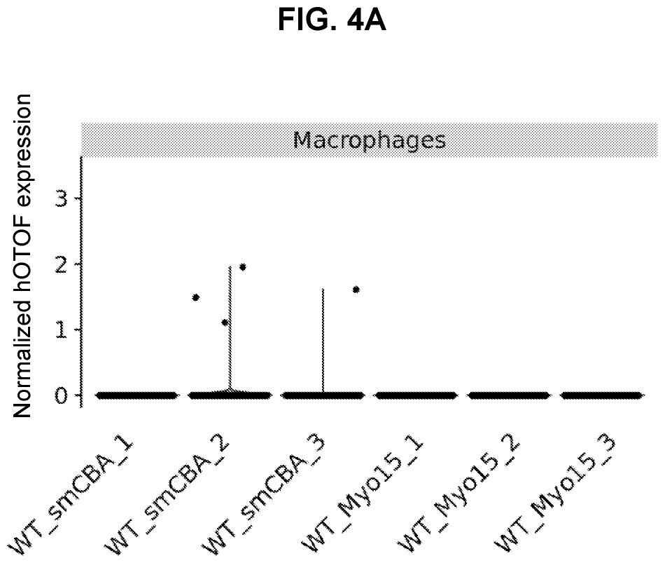

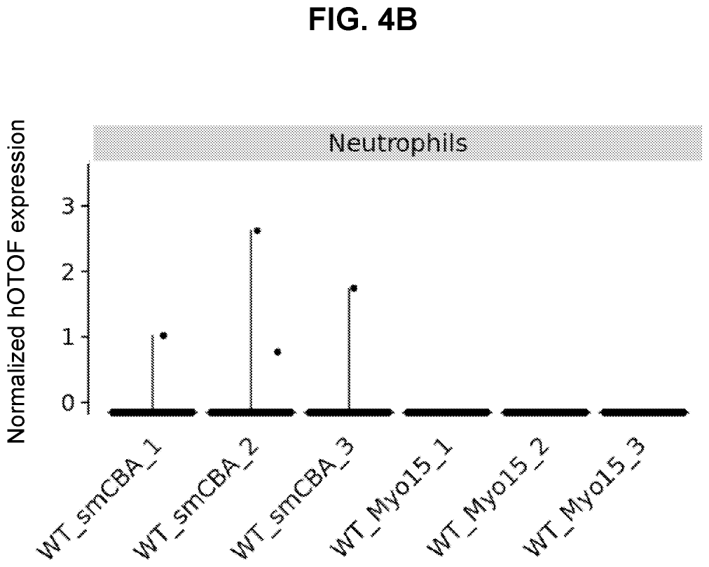

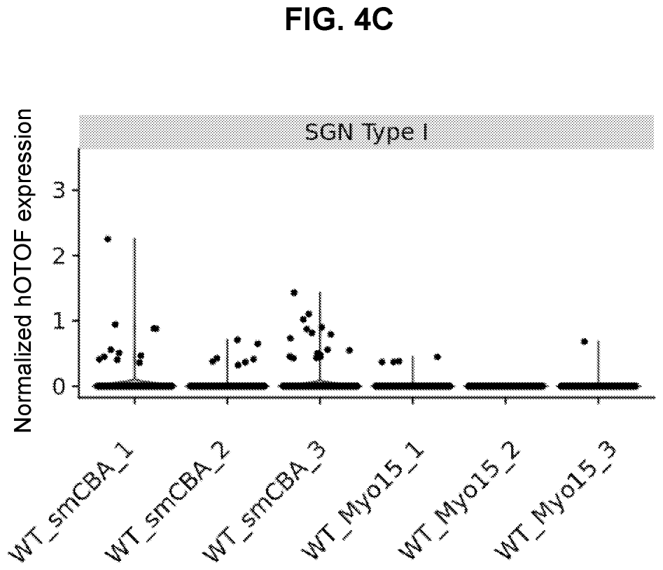

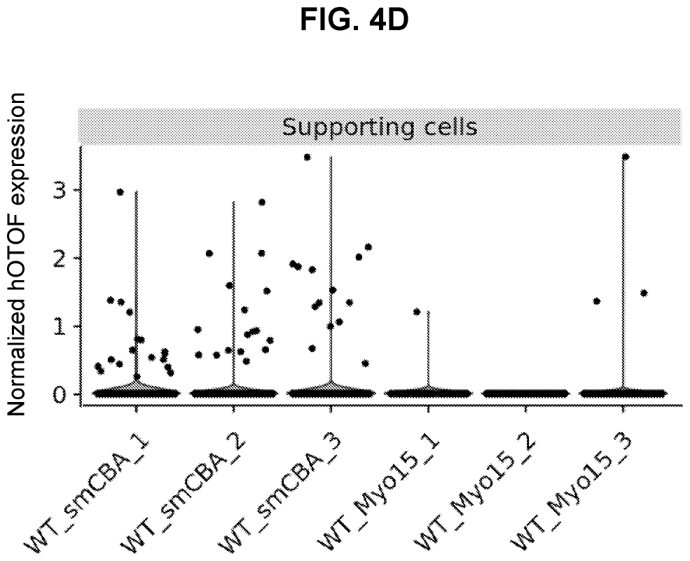

Furthermore, off-target expression of vector-encoded transgenes was analyzed for human otoferlin (hOTOF)-encoding transgenes in both AAV-smCBA and AAV-Myo15-treated samples in macrophages (FIG. 4A), neutrophils (FIG. 4B), spiral ganglion type I neurons (SGN Type I; FIG. 4C), and cochlear supporting cells (FIG. 4D). This analysis revealed increased off-target expression in immune cells in AAV-smCBA-treated samples as compared to AAV-Myo15-treated samples.

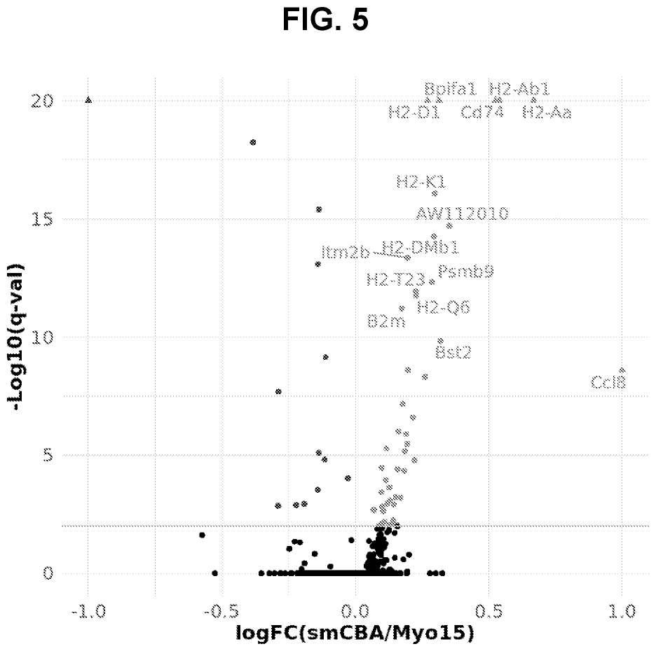

In addition, differential expression of multiple genes was identified between macrophages from cochlea treated with AAV-smCBA or AAV-Myo15, many of which are known to be involved in cell-mediated antigen presentation, such genes including C-C motif chemokine ligand 8 (CCL8), bone marrow stromal cell antigen 2 (BST2), beta-2-microglobulin (B2M), histocompatibility 2, Q region locus 6 (H2-Q6), histocompatibility 2, T region locus 23 (H2-T23), proteasome 20S subunit beta 9 (PSMB9), integral membrane protein 2B (ITM2B), histocompatibility 2, class II, locus MB1 (H2-DMB1), small secreted protein interferon-induced (AW112010), histocompatibility 2, K region locus 1 (H2-K1), histocompatibility 2, D region locus 1 (H2-D1), CD74 molecule (CD74), histocompatibility 2, class II antigen A (H2-AA), BPI fold containing family A member 1 (BPIFA1), and histocompatibility 2, class II antigen A, beta 1 (H2-AB1) (FIG. 5).

### Example 2—Altered Ligand-Receptor Interactions in Cells Isolated from the Inner Ear of Mice Treated with an AAV Vector Containing a Ubiquitous Promoter

To determine if treatment of administration of an AAV vector containing a ubiquitous promoter to the inner ear alters ligand-receptor interactions between cells, the following methods were performed.

### Ligand-Receptor Interactions Identification

The CellphoneDB method with default parameters was used to identify significant ligand-receptor interactions between pairs of cell types across single cell samples. The cell type labels were obtained from the strategy outlined in Example 1. Ligand-receptor interaction identification was performed on 5,000 cell downsamplings of each sample. Mouse gene IDs were converted to human IDs using biomaRt, and only 1-1 mappings were used.

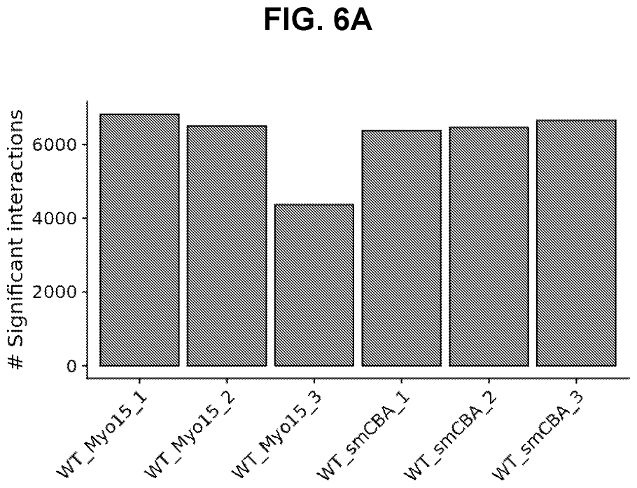

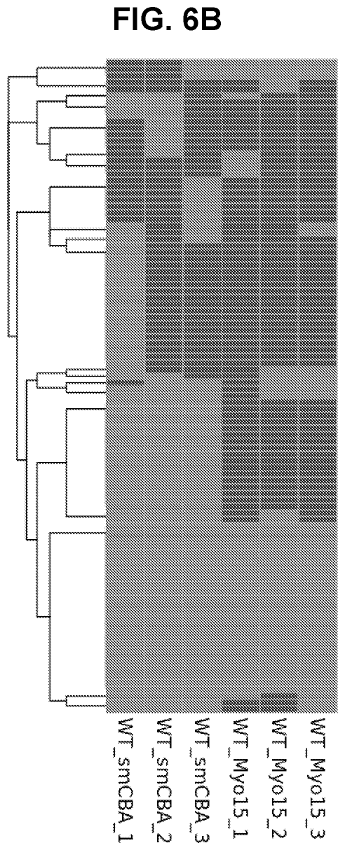

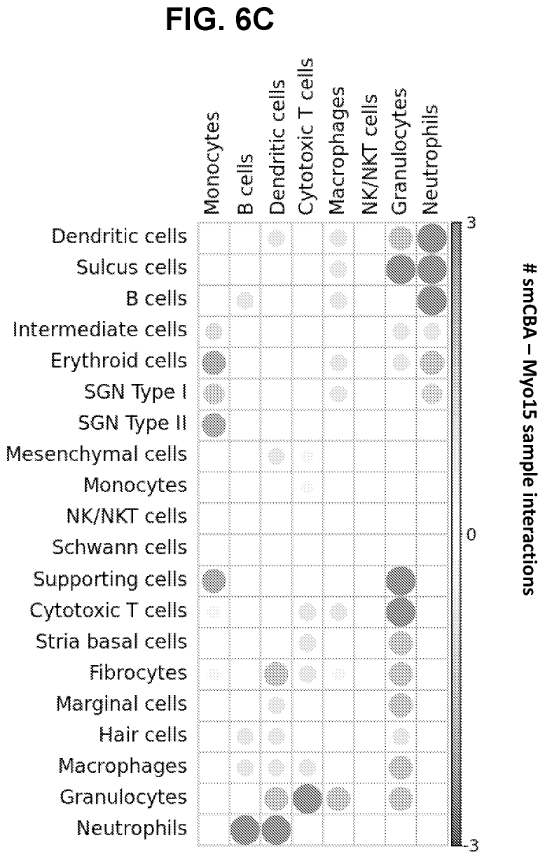

Significant ligand-receptor interactions were identified in three samples for each of the samples treated with AAV-smCBA and AAV-Myo15 (FIG. 6A). Over 4,000 significant interactions were found in each sample. Of those, more than 1,300 interactions common to all smCBA samples were not significant across AAV-Myo15 samples, compared to only about 500 Myo15-biased interactions. In particular, tumor necrosis factor receptor super family (TNFRSF) ligand-receptor interactions appeared to be significantly different between AAV-smCBA- and AAV-Myo15-treated samples (FIG. 6B). Analysis of distribution of ligand-receptor interactions across cell type pairs revealed that the significant interactions primarily involved neutrophils and granulocytes (FIG. 6C).

### Example 3—Administration of a Composition Containing a Nucleic Acid Vector Containing Therapeutic Polynucleotide Operably Linked to a Ubiquitous Promoter in Combination with an Inhibitor of Inflammatory or Immune Signaling

According to the methods disclosed herein, a physician of skill in the art can treat a subject, such as a human, with an inner ear disorder (e.g., hearing loss, tinnitus, or vestibular dysfunction) so as to improve or restore inner ear function. To this end, a physician of skill in the art can administer to the human patient a composition containing an AAV vector (e.g., an AAV1, AAV2, AAV2quad(Y-F), AAV3, AAV4, AAV5, AAV6, AAV7, AAV8, AAV9, Anc80, 7m8, PHP.B, PHP.eB, or PHP.S vector) containing a therapeutic polynucleotide operably linked to a ubiquitous promoter (e.g., an H1 promoter, a 7SK promoter, an apolipoprotein E-human α1-antitrypsin promoter (hAAT), a CK8 promoter, a murine U1 promoter (mU1a), an elongation factor 1α (EF-1α) promoter, an early growth response 1 (EGR1) promoter, a thyroxine binding globulin (TBG) promoter, a phosphoglycerate kinase (PGK) promoter, a CAG promoter, a chicken β-actin (CBA) promoter, an smCBA promoter, a CB7 promoter, a hybrid CMV enhancer/human β-actin promoter, a human β-actin promoter, a cytomegalovirus (CMV) promoter (e.g., the CMV immediate-early enhancer and promoter, a CMVmini promoter, a minCMV promoter, a CMV-TATA+INR promoter, or a min CMV-T6 promoter), a CASI promoter, a dihydrofolate reductase (DHFR) promoter, a murine mammary tumor virus LTR promoter, an adenovirus major late (Ad MLP) promoter, a β-globin promoter (e.g., a minimal β-globin promoter), an HSV promoter (e.g., a minimal HSV ICPO promoter or a truncated HSV ICPO promoter), an SV40 promoter (e.g., an SV40 minimal promoter or an SV40 early promoter), a rous sarcoma virus (RSV) promoter, an eukaryotic translation initiation factor 4A1 (EIF4A1) promoter, a ferritin heavy (FerH) promoter, a ferritin light (FerL) promoter, a glyceraldehyde-3-phospohate dehydrogenase (GAPDH) promoter, a heat shock protein family A member 5 (HSPA5) gene, a heat shock protein family A member 4 (HSPA4) promoter, a ubiquitin B (UBB) promoter, or a U6 promoter) in combination with an inhibitor of inflammatory or immune signaling (e.g., an anti-inflammatory agent, inhibitor of cell-mediated immunity, or cellular de-targeting agent described herein). The composition containing the AAV vector and the inhibitor of inflammatory or immune signaling may be administered to the patient, for example, by local administration to the inner ear (e.g., injection into a semicircular canal or to or through the round window membrane), or the agents may be administered by combined local and systemic administration (e.g., the nucleic acid vector encoding the therapeutic polynucleotide is administered locally to the inner ear and the inhibitor of inflammatory or immune signaling is administered systemically) to treat inner ear dysfunction.

Following administration of the composition to a patient, a practitioner of skill in the art can monitor the expression of the therapeutic agent (e.g., an inner ear protein, peptide, antibody or antigen-binding fragment thereof, RNAi sequence, miRNA, or a nuclease) encoded by the transgene, and the patient's improvement in response to the therapy, by a variety of methods. For example, a physician can monitor the patient's hearing function using standard tests such as audiometry, auditory brainstem response (ABR), electrocochleography (ECOG), and otoacoustic emissions. In another example, the physician can monitor the patient's vestibular function by performing standard tests such as electronystagmography, video nystagmography, VOR tests (e.g., head impulse tests (Hairnagyi-Curthoys test, e.g., VHIT), or caloric reflex tests), rotation tests, vestibular evoked myogenic potential, or computerized dynamic posturography. A finding that the patient exhibits improved hearing function or vestibular function in one or more of the tests following administration of the composition compared to test results obtained prior to administration of the composition indicates that the patient is responding favorably to the treatment. Subsequent doses can be determined and administered as needed.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Other embodiments are in the claims.

1. A method of reducing nucleic acid vector-induced toxicity in the inner ear of a subject, the method comprising administering to the subject an effective amount of (a) a nucleic acid vector comprising a ubiquitous promoter operably linked to a polynucleotide encoding a therapeutic agent; and (b) an inhibitor of inflammatory or immune signaling, wherein the nucleic acid vector is locally administered to the middle or inner ear.

2. A method of reducing off-target transduction of immune cells in the inner ear, the method comprising administering to a mixed population of inner ear cells and immune cells an effective amount of (a) a nucleic acid vector comprising a ubiquitous promoter operably linked to a polynucleotide encoding a therapeutic agent; and (b) an inhibitor of inflammatory or immune signaling.

3. A method of improving therapeutic efficacy of a nucleic acid vector in an inner ear of a subject, the method comprising administering to the subject an effective amount of (a) a nucleic acid vector comprising a ubiquitous promoter operably linked to a polynucleotide encoding a therapeutic agent; and (b) an inhibitor of inflammatory or immune signaling, wherein the nucleic acid vector is locally administered to the middle or inner ear.

4. A method of treating an inner ear dysfunction in a subject in need thereof, the method comprising administering to the subject an effective amount of (a) a nucleic acid vector comprising a ubiquitous promoter operably linked to a polynucleotide encoding a therapeutic agent; and (b) an inhibitor of inflammatory or immune signaling, wherein the nucleic acid vector is locally administered to the middle or inner ear.

5. A method of reducing immune cell number and/or activity in an inner ear of a subject, the method comprising administering to the subject an effective amount of (a) a nucleic acid vector comprising a ubiquitous promoter operably linked to a polynucleotide encoding a therapeutic agent; and (b) an inhibitor of inflammatory or immune signaling.

6. The method of any one of claims 1-5, wherein the nucleic acid vector is a viral vector.

7. The method of claim 6, wherein the viral vector is an adeno-associated viral (AAV) vector.

8. The method of claim 7, wherein the AAV vector has an AAV1, AAV2, AAV2quad(Y-F), AAV3, AAV4, AAV5, AAV6, AAV7, AAV8, AAV9, AAV10, AAV11, rh10, rh39, rh43, rh74, Anc80, Anc80L65, DJ/8, DJ/9, 7m8, PHP.B, PHP.eb, or PHP.S capsid.

9. The method of any one of claims 1-8, wherein the ubiquitous promoter is an H1 promoter, a 7SK promoter, an apolipoprotein E-human α1-antitrypsin promoter (hAAT), a CK8 promoter, a murine U1 promoter (mU1a), an elongation factor 1α (EF-1α) promoter, an early growth response 1 (EGR1) promoter, a thyroxine binding globulin (TBG) promoter, a phosphoglycerate kinase (PGK) promoter, a CAG promoter, a chicken β-actin (CBA) promoter, an smCBA promoter, a CB7 promoter, a hybrid CMV enhancer/human β-actin promoter, a human β-actin promoter, a cytomegalovirus (CMV) promoter, a CASI promoter, a dihydrofolate reductase (DHFR) promoter, a murine mammary tumor virus LTR promoter, an adenovirus major late (Ad MLP) promoter, a β-globin promoter, an HSV promoter, an SV40 promoter, a rous sarcoma virus (RSV) promoter, an eukaryotic translation initiation factor 4A1 (EIF4A1) promoter, a ferritin heavy (FerH) promoter, a ferritin light (FerL) promoter, a glyceraldehyde-3-phospohate dehydrogenase (GAPDH) promoter, a heat shock protein family A member 5 (HSPA5) gene, a heat shock protein family A member 4 (HSPA4) promoter, a ubiquitin B (UBB) promoter, or a U6 promoter.

10. The method of any one of claims 1-9, wherein the therapeutic agent is an inner ear protein, a peptide, an antibody or antigen-binding fragment thereof, an inhibitory nucleic acid, a microRNA, or a component of a gene editing system.

11. The method of claim 10, wherein the inner ear protein is a protein that is natively expressed by inner ear cells.

12. The method of claim 10, wherein the inhibitory nucleic acid is a short hairpin RNA (shRNA), microRNA-adapted shRNA (shmiRNA), or an antisense oligonucleotide (ASO).

13. The method of claim 10, wherein the component of a gene editing system is a zinc finger nuclease (ZFN), transcription activator-like effector nuclease (TALEN), or clustered regularly interspaced short palindromic repeats (CRISPR) nuclease.

14. The method of claim 13, wherein the CRISPR nuclease is a CRISPR-Cas9 or CRISPR-Cas12 nuclease, wherein the CRISPR nuclease further comprises a guide RNA (gRNA) sequence.

15. The method of any one of claims 1-14, wherein the inhibitor of inflammatory or immune signaling is an anti-inflammatory agent, an inhibitor of cell-mediated immunity, or a cellular de-targeting agent.

16. The method of claim 15, wherein the inhibitor of cell-mediated immunity is an inhibitory nucleic acid or a nuclease comprising a gRNA having a sequence that is complementary to a sequence of a gene selected from the group consisting of TNF receptor superfamily member 1A or 1B (TNFRSF1 A/B), TNF receptor superfamily member 13A or 13B (TNFRSF13 A/B), C-C motif chemokine ligand 8 (CCL8), bone marrow stromal cell antigen 2 (BST2), beta-2-microglobulin (B2M), histocompatibility 2, Q region locus 6 (H2-Q6), histocompatibility 2, T region locus 23 (H2-T23), proteasome 20S subunit beta 9 (PSMB9), integral membrane protein 2B (ITM2B), histocompatibility 2, class II, locus MB1 (H2-DMB1), small secreted protein interferon-induced (AW112010), histocompatibility 2, K region locus 1 (H2-K1), histocompatibility 2, D region locus 1 (H2-D1), CD74 molecule (CD74), histocompatibility 2, class II antigen A (H2-AA), BPI fold containing family A member 1 (BPIFA1), histocompatibility 2, class II antigen A, beta 1 (H2-AB1), CD86 molecule (CD86), and C-X-C motif chemokine receptor 3 (CXCR3).

17. The method of claim 15, wherein the cellular de-targeting agent is nucleic acid sequence targeted by a microRNA expressed in an immune cell.

18. The method of claim 17, wherein the nucleic acid sequence targeted by a microRNA expressed in an immune cell is comprised in the nucleic acid vector encoding the therapeutic agent.

19. The method of claim 17 or 18, wherein the microRNA expressed in an immune cell is miR-9, miR-15a/16, miR-21, miR-23a, miR-24, miR-29a, let-7, miR-98, miR-106a, miR-125a-99b-let-7e cluster, miR-125b, miR-126, miR-127, miR-142, miR-145, miR-146a/b, miR-147b, miR-150, miR-155, miR-181, miR-187, miR-212, miR-222, miR-223, miR-451, miR-511, miR-720, miR-886-5p, and miR-4661, hsa-miR-378_st, hsa-miR-31_st, hsa-miR-935_st, hsa-miR-143_st, hsa-miR-362-5p_st, hsa-miR-532-5p_st, hsa-miR-500-star_st, hsa-miR-663_st, hsa-miR-125a-5p_st, hsa-miR-150_st, HBII-239_st, HBII-429_st, HBII-202_st, U27_st, U95_st, hsa-miR-768-5p_st, hsa-miR-223_st, or hsa-miR-652_st.

20. The method of claim 15, wherein the cellular de-targeting agent is an inhibitory nucleic acid having complementarity to a transduction-permissive gene, or a nuclease comprising a gRNA having complementarity to a transduction permissive gene, or a polynucleotide encoding the same.

21. The method of claim 16 or 20, wherein the polynucleotide encoding the inhibitory nucleic acid or nuclease is operably linked to an immune-cell specific promoter.

22. The method of claim 16 or 20, wherein the inhibitory nucleic acid inhibitor of inflammatory or immune signaling is a naked nucleic acid.

23. The method of claim 16 or 20, wherein the inhibitory nucleic acid or the nuclease is encoded in the nucleic acid vector comprising the ubiquitous promoter.

24. The method of claim 16 or 20, wherein the inhibitory nucleic acid or the nuclease is encoded in a second nucleic acid vector, and wherein the polynucleotide encoding the inhibitory nucleic acid or nuclease is operably linked to an immune cell-specific promoter.

25. The method of claim 1, wherein the nucleic acid vector-induced toxicity is inflammation-induced toxicity or cell-mediated immunity-induced toxicity.

26. The method of claim 1 or claim 25, wherein the reducing nucleic acid vector-induced toxicity comprises reducing nucleic acid vector-induced toxicity by 5%, 10%, 15%, 20%, 25%, 30%, 35%, 40%, 45%, 50%, 55%, 60%, 65%, 70%, 75%, 80%, 85%, 90%, 95%, 99% or more as compared to nucleic acid vector-induced toxicity in a subject administered the nucleic acid vector in the absence of an inhibitor of inflammatory or immune signaling.

27. The method of claim 2, wherein the mixed population of inner ear cells comprises inner ear hair cells, inner ear supporting cells, spiral ganglion neurons, Scarpa's ganglion neurons, Claudius cells, spiral prominence cells, root cells, interdental cells, basal cells of the stria vascularis, intermediate cells of the stria vascularis, marginal cells of the stria vascularis, endothelial cells of cochlear capillaries, cochlear fibrocytes, cells of Reissner's membrane, and cochlear glial cells.

28. The method of claim 2, wherein the off-target transduction of inner ear immune cells is reduced by 5%, 10%, 15%, 20%, 25%, 30%, 35%, 40%, 45%, 50%, 55%, 60%, 65%, 70%, 75%, 80%, 85%, 90%, 95%, 99% or more as compared to compared to a mixed population of inner ear cells and immune cells treated with the nucleic acid vector in the absence of an inhibitor of inflammatory or immune signaling.

29. The method of claim 3, wherein the improving therapeutic efficacy comprises preventing or reducing hearing loss, preventing or reducing tinnitus, delaying development of hearing loss, slowing the progression of hearing loss, improving hearing, improving balance, reducing dizziness, reducing vertigo, delaying development of vestibular dysfunction, slowing the progression of vestibular dysfunction, increasing expression and/or activity of the therapeutic agent in one or more inner ear cells, increasing inner ear hair cell development, increasing inner ear hair cell numbers, increasing or inducing inner ear hair cell maturation, increasing inner ear hair cell survival, increasing inner ear hair cell regeneration, improving inner ear hair cell function, improving inner ear supporting cell function, improving inner ear supporting cell proliferation, improving inner ear supporting cell maturation, increasing inner ear supporting cell numbers, or increasing inner ear supporting cell survival.

30. The method of claim 27 or 29, wherein the inner ear hair cells are cochlear inner hair cells (IHC), cochlear outer hair cells (OHC), type I vestibular hair cells, or type II vestibular hair cells.

31. The method of claim 27 or 29, wherein the inner ear supporting cells are cochlear supporting cells or vestibular supporting cells.

32. The method of claim 31, wherein the cochlear supporting cells comprise Border cells, inner phalangeal cells, inner pillar cells, outer pillar cells, first row Deiters' cells, second row Deiters' cells, third row Deiters' cells, and/or Hensen's cells.

33. The method of claim 4, wherein the inner ear dysfunction is hearing loss.

34. The method of claim 33, wherein the hearing loss is genetic hearing loss.

35. The method of claim 34, wherein the genetic hearing loss is autosomal dominant hearing loss, autosomal recessive hearing loss, or X-linked hearing loss.

36. The method of claim 33, wherein the hearing loss is acquired hearing loss.

37. The method of claim 36, wherein the acquired hearing loss is noise-induced hearing loss, age-related hearing loss, disease or infection-related hearing loss, head trauma-related hearing loss, or ototoxic drug-induced hearing loss.

38. The method of claim 37, wherein the ototoxic drug is selected from the group consisting of aminoglycosides, antineoplastic drugs, ethacrynic acid, furosemide, salicylates, and quinine.

39. The method of claim 4, wherein the inner ear dysfunction is tinnitus.

40. The method of claim 4, wherein the inner ear dysfunction is vestibular dysfunction.

41. The method of claim 40, wherein the vestibular dysfunction is vertigo, dizziness, loss of balance, bilateral vestibulopathy, oscillopsia, or a balance disorder.

42. The method of claim 5, wherein the immune cells are monocytes and/or dendritic cells.

43. The method of claim 5 or claim 42, wherein reducing a number of immune cells in the inner ear of a subject comprises reducing the number of immune cells in the inner ear by 5%, 10%, 15%, 20%, 25%, 30%, 35%, 40%, 45%, 50%, 55%, 60%, 65%, 70%, 75%, 80%, 85%, 90%, 95%, 99% or more as compared to the number of inner ear immune cells in a subject treated with the nucleic acid vector in the absence of an inhibitor of inflammatory or immune signaling.

44. The method of any one of claims 5, 42, or 43, wherein the reducing a number and/or activity of immune cells comprises reducing immune cell recruitment to the inner ear, increasing immune cell death in the inner ear, reducing immune cell migration in the inner ear, reducing activation of immune cells, reducing phagocytosis by immune cells, reducing antibody-dependent cellular cytotoxicity by immune cells, reducing immune cell polarization, reducing immune cell proliferation, reducing immune cell differentiation, reducing immune cell cytokine production, reducing immune cell antigen presentation, reducing immune cell maturation, or reducing immune cell degranulation.

45. The method of any one of claims 1-44, wherein the administering comprises simultaneous administration of the nucleic acid vector and the inhibitor of inflammatory or immune signaling to the subject or to the mixed population of inner ear cells and immune cells.

46. The method of claim 45, wherein the simultaneous administration comprises simultaneous local administration to the middle or inner ear of both the nucleic acid vector encoding the therapeutic agent and the inhibitor of inflammatory or immune signaling.

47. The method of claim 45, wherein the simultaneous administration comprises simultaneous local administration of the nucleic acid vector encoding the therapeutic agent to the middle or inner ear and systemic administration of the inhibitor of inflammatory or immune signaling.

48. The method of any one of claims 1-44, wherein the administering comprises sequential administration of the nucleic acid vector encoding the therapeutic agent and the inhibitor of inflammatory or immune signaling, wherein the sequential administration comprises:
a) administration of the nucleic acid vector encoding the therapeutic agent prior to administration of the inhibitor of inflammatory or immune signaling; or
b) administration of the nucleic acid vector encoding the therapeutic agent following administration of the inhibitor of inflammatory or immune signaling.

49. The method of claim 48, wherein the sequential administration comprises local administration to the middle or inner ear of both the nucleic acid vector and the inhibitor of inflammatory or immune signaling.

50. The method of claim 48, wherein the sequential administration comprises local administration of the nucleic acid vector encoding the therapeutic agent to the middle or inner ear and systemic administration of the inhibitor of inflammatory or immune signaling.

51. The method of any one of claims 1, 3-5, 46, 47, 49, and 50, wherein local administration to the middle or inner ear comprises administration to a semicircular canal, transtympanic administration, intratympanic administration, administration into the perilymph, administration into the endolymph, administration to or through the round window, or administration to or through the oval window.

52. The method of any one of claims 47-51, wherein systemic administration comprises intravenous, intramuscular, subcutaneous, intraperitoneal, transmucosal, or oral administration.

53. The method of any one of claims 1-52, wherein the subject is a human.
